(19) United States
(12) Patent Application Publication
(10) Pub. No.: US 2021/0102237 A1
(43) Pub. Date: Apr. 8, 2021

(54) MEANS AND METHODS FOR NUCLEIC ACID TARGET DETECTION

(71) Applicant: Biotangents Limited, Penicuik (GB)

(72) Inventors: Lina GASIUNAITE, Penicuik (GB); Andrew Mark HALL-PONSELE, Penicuik (GB); David PAGE, Penicuik (GB); Archana NAYAK, Penicuik (GB)

(21) Appl. No.: 16/977,339

(22) PCT Filed: Feb. 28, 2019
(86) PCT No.: PCT/GB2019/050568

§ 371 Date: Sep. 1, 2020

(30) Foreign Application Priority Data
Mar. 1, 2018 (GB) 1803367.0

(51) Int. Cl. C12Q 1/682; C12Q 1/6897

(52) U.S. Cl. CPC C12Q 1/682; C12Q 1/6897

(57) ABSTRACT

The present invention generally relates to a method, reaction components and apparatus for facilitating detection of a nucleic acid target sequence by combining a target nucleic acid detection system that involves the creation of a three-way junction capable of producing an oligonucleotide signal molecule with riboregulator switch-mediated detection

### FIELD OF THE INVENTION

The present invention generally relates to detection of a nucleic acid target sequence. More specifically, the present invention relates to a method, reaction components and apparatus for facilitating detection of a nucleic acid target sequence using a three-way junction (3WJ) complex capable of generating an oligonucleotide signaling sequence, which acts as a primer for amplification of a trigger oligonucleotide or which can by itself trigger activation of a riboregulator switch. The method can be carried out using suitable apparatus, including portable devices. For example, the apparatus could be a microfluidic device or it could be paper-based, e.g. card, onto which a fluid test sample is applied, triggering the reaction and yielding a detectable signal. The detectable signal could be a visible colour change.

### BACKGROUND TO THE INVENTION

Nucleic acid target sequence detection is routinely used in many medical, veterinary and research applications. For example, it may be used to diagnose a particular disease or detect the presence of a particular infectious agent (e.g. bacterium, virus and the like). It may also be used in personalised medicine to identify patients most likely to respond to a particular therapy based on the patient's genetic make-up. It may also be used in a variety of research studies that involve understanding specific allele sequences present in an individual or model organism. The conventional strategy to detect nucleic acid target sequences is to use amplification reactions (such as those based on polymerase chain reaction (PCR)) in methods that require expensive machinery and/or the use of expensive materials such as fluorescent probes, and are typically carried out within the laboratory setting away from the subject (patient/animal) being tested.

There is an increasing need for reliable and inexpensive tools to diagnose diseases of animals (including livestock, farmed aquatic animals, pedigree animals and companion animals), that can be used in the field (outside of a laboratory setting).

Current diagnostic tests work by direct identification of the disease agent (e.g. through cell culture of the pathogen) or indirect observation of the host (e.g. by measuring an immune response to the pathogen over several days or through monitoring of symptoms). These tests are frequently slow, costly to perform, require the use of a dedicated diagnostics laboratory, and lack sensitivity. Furthermore, many tests for pathogens are unable to differentiate infected and vaccinated states, a major contributing factor to the lack of successful vaccination programmes for several costly animal diseases.

Tests involving the generation of a three-way junction (3WJ) are known. A 3WJ is a three-nucleic acid complex formed when parts of two nucleic acid probes hybridise to a target sequence in an adjacent or substantially adjacent manner and the other parts of the two probes hybridise to themselves.

The use of a 3WJ to detect a particular target sequence and create a signaling oligonucleotide that can then be detected, is utilised in the signal-mediated amplification of RNA technique (SMART), as described in WO 93/06240. This system generates an RNA oligonucleotide signal sequence as a result of RNA transcription from an RNA promoter created by an extension reaction from the 3WJ. WO9937806 (Cytocell Ltd.) adapts this process by incorporation of one or more destabilising moieties into the probes used that prevent hybridisation of the first and second probes in the absence of the sequence of interest.

WO9937805 (Cytocell Ltd.) utilises SMART by creating a functional double-stranded RNA polymerase promoter when the 3WJ assembles. A complex comprising three strands of nucleic add: a target sequence, a first probe and a second probe is formed; The first probe comprises a sequence complementary to the target sequence and the full-length sequence of a first strand of a double-stranded promoter. The second probe comprises a sequence complementary to the target sequence and a part of the second strand of the double-stranded promoter which is complementary to a part of the first strand. The other part of the promoter sequence is provided by the target sequence, such that when the complex of three nucleic acids (3WJ) has assembled a substantially functional RNA promoter is formed. RNA polymerase can then cause de novo synthesis of nucleic acid from the promoter using the single-stranded template sequence provided by the other probe. In this system, there is no extension required for promoter formation.

SMART has previously been demonstrated for the detection of E coli genomic DNA and ribosomal RNA, even using crude E coli cell lysates without sophisticated sample preparation. When probing for the E coli 23S ribosomal RNA sequence, the method detected as little as 4 pM single-stranded synthetic target (Wharam et al., Nucleic Acids Research, 29:e54, 2001). Published accounts of the SMART technique report a total assay time of 2.5-5 hours (Hall et al., BioTechniques, 32:604-611, 2002; Levi et al., Journal of Clinical Microbiology, 41:3187-3191, 2003; Wharam et al., Virology Journal, 4, 52, 2007). However, the signal RNA in these examples was detected by enzyme-linked oligosorbent assay in a separate step.

SMART has also been applied to detect DNA and messenger RNA of the marine cyanophage virus (Hall et al., 2002, ibid), and, in a different study, a commercially-available kit (CytAMP®) based on SMART was used to detect methicillin-resistant Staphylococcus aureus (MRSA) in lysed clinical bacterial isolates (Levi et al., 2003, ibid). The specificity and sensitivity of the method for MRSA detection was comparable to PCR.

Murakami, T et al., (Nucleic Acids Research. 40:e22, 2012), combined 3WJ formation with primer generation-rolling circle amplification (PG-RCA) in an isothermal reaction format to detect an RNA target sequence. Primer extension from one of the binding probes using the second probe as template generates a restriction enzyme site (e.g. for Nb.Bsml), as opposed to a functional promoter in SMART, and then signal sequence. The restriction enzyme then nicks the double-stranded extension molecule and the oligonucleotide “signal sequence” is then dissociated from the complementary sequence. Repeated primer extension, nicking and dissociation can create more signal sequence molecules which are then exponentially amplified in situ by PG-RCA.

A close alternative to SMART is Nucleic Acid Sequence-Based Amplification (NASBA), inspired by a retroviral strategy of RNA replication through the generation of complementary DNA intermediates. It is well suited to amplifying RNA targets 10⁶ to 10⁹-fold in 90 minutes. NASBA has been coupled to different methods of RNA reporting, such as electro-chemiluminescence and enzyme-linked gel assay, requiring different steps for nucleic acid amplification and reporting.

There have been attempts to integrate nucleic acid amplification by NASBA and detection in a single tube. For instance, a combination of NASBA and the RNA aptamer Spinach.ST (Pothoulakis et al., ACS Synth. Biol., 3:182-187, 2014) was tested by Bhadra and Ellington (RNA 20:1012, 2014). This combination was able to detect and amplify target RNA relatively quickly (1 h), but sensitivity was low—a minimal 10 nM concentration of target RNA was detectable with a 2:1 signal-to-noise ratio, making it in the order of 1,000 times less sensitive than SMART. Researchers hypothesised that the efficiency of reverse transcription was one of the limiting factors.

The present invention, combining a 3WJ (such as SMART method) for signal amplification with a riboregulator switch for signal reporting, does not involve reverse transcription, as required for the NASBA technique. This omission is expected to allow lower concentrations of RNA to be detected more quickly.

A riboregulator (or riboregulator switch) is a ribonucleic acid (RNA) that can be used to repress or activate translation of an open reading frame and thus production of a protein.

They were inspired by natural bacterial small RNA. They incorporate hairpin structures that block the access of the ribosome to the mRNA transcript and prevent the translation of the downstream protein coding sequence. Translation requires a trans-activating trigger oligonucleotide (e.g. RNA molecule) molecule to anneal to a complementary sequence within the hairpin loop. The hairpin loop then undergoes conformational changes resulting in the ribosome binding site (RBS) becoming accessible to ribosome binding and subsequent translation of the downstream protein coding sequence.

Green et al. (Cell, 159:925-939, 2014) improved riboregulator design by incorporating a toehold domain for more efficient RNA-RNA interactions. In this strategy, a toehold region complementary to the trigger RNA is exposed from the hairpin structure, while the RBS is located in the hairpin loop. Green et al. designed RNA sequences that would fold into specific structures, with final variants exhibiting up to a 400-fold dynamic response to the trigger RNA under experimental conditions. This toehold strategy is an improvement on the original riboregulator design as it eliminates the need for sequence conservation between the RBS and the target sequence, so giving greater flexibility over the choice of target sequence.

WO 2014/074648 (President and Fellows of Harvard College and Trustees of Boston University) describes programmable toehold riboregulators (also referred to as toehold switches) that can be activated by RNAs.

### SUMMARY OF THE INVENTION

The present invention combines oligonucleotide signal generation from a 3WJ (such as SMART) with a more flexible RNA reporting system comprising a riboregulator switch (e.g. one containing a toehold domain) containing and controlling a reporting unit (such as lacZ). The combination of 3WJ (e.g. via SMART) with riboregulator switch-based detection is novel. Furthermore, it allows for detection of an RNA or DNA target sequence, which is not possible with NASBA technique. The system also allows for use of an optimal and universal riboregulator switch design even if the target is changed. This is achieved by having the signal oligonucleotide created from the 3WJ (with or without the optional amplification of signal oligonucleotide sequence), act as the trigger for the riboregulator switch, rather than using the amplified target sequence as the trigger. Since the design of the riboregulator switch need not change with the target, the placement of the RBS within the riboregulator switch structure is less important, allowing us to combine the features of both, riboregulator and a toehold switch if needed.

A SMART assay consists of two single-stranded oligonucleotide probes (extension and template): each probe includes one region that can hybridise to the target (at adjacent sites) and another, much shorter, region that hybridises to the other probe. The two probes are designed such that they can only anneal to each other in the presence of the specific target, so forming a 3WJ (e.g. FIG. 3A). Following 3WJ formation, a DNA polymerase extends the extension probe using the opposing probe as template to produce a double- stranded RNA polymerase promoter (e.g. T7) sequence (e.g. FIG. 3B). The assay relies on the fact that only the double-stranded promoter is fully functional. RNA polymerase can then generate multiple copies of an RNA signal. The signal is therefore target dependent, being produced only when a specific target is present to allow 3WJ formation. The RNA signal may itself be amplified. For example, the RNA signal can bind to a second template oligonucleotide (probe for RNA amplification) which is then extended by DNA polymerase to generate a double-stranded RNA polymerase promoter, leading to transcription which increases the RNA yield, so improving the sensitivity of the assay (Wharam et al., 2001, ibid).

SMART is only one of the techniques that creates and utilises a 3WJ. Any 3WJ method that generates and releases a single-stranded oligonucleotide can be used in the invention.

According to one aspect of the invention there is provided a method of detecting a nucleic acid sequence of interest in a sample, the method comprising (a) contacting the sample with first and second probes capable of creating a three-way junction when the target sequence is present in the sample wherein the first probe comprises a portion substantially complementary to the sequence of interest and so capable of hybridising thereto and a portion non-complementary to the sequence of interest but comprising a sequence complementary to the second probe and a template signal sequence, and wherein the second probe comprises a portion substantially complementary to the sequence of interest and so capable of hybridising thereto and a portion non-complementary to the sequence of interest but complementary to a part of that portion of the first probe which is non-complementary to the sequence of interest, such that the first and second probes are capable of hybridising to the sequence of interest in an adjacent or substantially adjacent manner so as to allow complementary portions of the first and second probes to hybridise to each other; (b) causing production of an oligonucleotide sequence that is complementary to the template signal sequence in the first probe; (c) bringing an oligonucleotide trigger sequence into contact with a riboregulator switch sequence, part of which is in the form of a hairpin loop structure, comprising an RNA sequence having single-stranded and double-stranded domains comprising a single-stranded domain capable of hybridising with part of the oligonucleotide trigger sequence, a RBS, an initiation codon and a coding domain for a reporter gene arranged such that the ribosome is only able to effect translation when the hairpin loop structure has been disrupted following binding of the oligonucleotide trigger sequence to the riboregulator switch; and (d) detecting the reporter gene product; wherein the presence of the reporter gene product indicates the presence of the sequence of interest in the sample; and, wherein the oligonucleotide trigger sequence is either the oligonucleotide sequence produced in step (b) or an oligonucleotide sequence produced using the oligonucleotide signal sequence produced in step (b) as an intermediate in an amplification reaction.

According to another aspect of the invention there is provided a method of detecting a nucleic acid sequence of interest in a sample, the method comprising (a) contacting the sample with first and second probes, wherein the first probe comprises a portion substantially complementary to the sequence of interest and so capable of hybridising thereto, and a portion non-complementary to the sequence of interest but comprising the full length sequence of a first strand of a double-stranded RNA promoter and a template signal sequence, and wherein the second probe comprises a portion substantially complementary to the sequence of interest and so capable of hybridising thereto, and a portion non-complementary to the sequence of interest but complementary to a part of that portion of the first probe which is non-complementary to the sequence of interest, such that the first and second probes are capable of hybridising to the sequence of interest in an adjacent or substantially adjacent manner, so as to allow complementary portions of the first and second probes to hybridise to each other; (b) causing extension of the second probe with a nucleic acid polymerase, using the first probe as a template so as to produce a functional RNA polymerase promoter; (c) causing production of an RNA signal sequence when the double-stranded functional RNA polymerase promoter produced in (b) is contacted with an RNA polymerase; (d) bringing a nucleic acid trigger sequence into contact with a riboregulator switch sequence, part of which is in the form of a hairpin loop structure comprising an RNA sequence having single-stranded and double-stranded domains comprising a single-stranded domain capable of hybridising part of the nucleic acid trigger sequence, a RBS, an initiation codon and a coding domain for a reporter gene arranged such that the ribosome is only able to effect translation when the hairpin loop structure has been disrupted following binding of the nucleic acid trigger sequence to the riboregulator switch; and (e) detecting the reporter gene product; wherein the presence of the reporter gene product indicates the presence of the sequence of interest in the sample; and, wherein the nucleic acid trigger sequence is either the signal sequence produced in step (c) or is a nucleic acid sequence produced when the signal sequence produced in step (c) is hybridised to an amplification probe capable of generating the nucleic acid trigger sequence.

In one embodiment, extension of the second probe with a nucleic acid polymerase also produces a double-stranded sequence that can be used by an RNA polymerase to create a single-stranded RNA signal sequence.

A toehold switch is an example of a suitable riboregulator switch. The important feature of the riboregulator switch is that binding of the oligonucleotide trigger sequence to the riboregulator switch effects a conformational change on the riboregulator switch such that the previously constrained RBS is accessible to the ribosome allowing translation of the reporter gene product. The oligonucleotide trigger sequence can therefore bind a toehold domain or any other part of the riboregulator switch, such as the loop of the hairpin. Suitable toehold switches for use in the present invention are disclosed in US20160312312.

The structure/conformation of the riboregulator switch is such that when in the inactive state the ribosome is unable to effect translation of the reporter gene sequence. In one embodiment, the RBS is not accessible to the ribosome. In another embodiment, the RBS is accessible but the ribosome cannot effect translation due to the conformation of the riboregulator switch. Upon activation, the hairpin loop structure is lost, the RBS becomes accessible to ribosome binding and the reporter gene product can then be produced. Activation generally occurs when an oligonucleotide hybridises to part of the single-stranded and part of the double-stranded region of the riboregulator switch. With riboregulator switches that have a toehold domain, the trigger oligonucleotide typically binds to the single-stranded toehold domain and part of the stem of the riboregulator sequence and this then leads to an alteration in the structure of the riboregulator switch which then permits ribosome binding and translation to produce the reporter gene product. In another embodiment, the trigger oligonucleotide binds to part of the loop domain of the riboregulator switch and part of the stem of the riboregulator switch sequence.

When the target sequence is present in a test sample, step (a) produces a three-way junction (3WJ) between the target sequence, the first probe and the second probe. An oligonucleotide signal sequence can be produced from the 3WJ.

In one embodiment, primer extension of the second probe using the first probe as template generates a double-stranded RNA polymerase promoter and RNA signal sequence, RNA polymerase can then bind the RNA promoter and transcribes the RNA signal sequence. In this way, multiple copies of the RNA signal sequence molecules are produced. Optionally, the number of RNA signal sequence molecules produced can be amplified by contacting the RNA signal sequence molecules with an amplification probe such that the RNA signal sequence molecule and amplification probe hybridise in such a way that primer extension can proceed from the 3′ end of the signal sequence using the amplification probe as a template to produce a functional double-stranded RNA promoter sequence and RNA signal sequence. RNA polymerase can then bind the double-stranded promoter and produce the RNA signal sequence molecules encoded within the 5′ end of the amplification probe.

Through appropriate design of the amplification probe multiple copies of the same RNA signal sequence molecule that was the primer sequence for the amplification probe or a second distinct RNA signal sequence molecule can be generated. In a particular embodiment, the most abundantly produced RNA signal sequence molecules serve as the trigger oligonucleotide sequence for the subsequent riboregulator switch activation.

The template probe may contain RNA signal sequence template or may contain the template for a complementary single-stranded DNA oligonucleotide. The template probe containing RNA signal sequence template may be used by RNA polymerase to transcribe RNA signal sequence either from single-stranded or from double-stranded probe; RNA signal sequence is complementary to RNA signal sequence template in the template probe. The extended Extension probe (non-template strand) is also complementary to RNA signal sequence template and is a DNA equivalent of RNA signal sequence.

As used herein, the term “RNA signal sequence” refers to a single-stranded RNA oligonucleotide. The term “oligonucleotide trigger sequence” or “nucleic acid trigger sequence” is a single-stranded DNA or RNA sequence molecule that is capable of hybridising to the riboregulator switch, effecting a conformational change that then result in production of the reporter gene product. An RNA signal sequence may be the oligonucleotide trigger sequence. The term “template signal sequence” is a sequence that is used as template for making the single-stranded oligonucleotide molecule that will act as the trigger oligonucleotide sequence for activating the riboregulator switch or as signal sequence for amplification of the trigger oligonucleotide. Typically, this is the sequence used as template by RNA polymerase to make a single-stranded RNA sequence.

As used herein, the riboregulator switch is also referred to as the riboregulator switch sequence or the riboregulator switch molecule. A toehold switch is a type of riboregulator switch.

In one embodiment, formation of the 3WJ generates a functional double-stranded RNA polymerase promoter. A functional double-stranded RNA promoter can be formed, for example, if the first three (5′) bases of the promoter sequence is complemented by three bases (e.g. 3′ ATT 5′) in the target sequence. An active RNA promoter is thus formed directly when the 3WJ forms. There is no extension required for promoter formation.

In a particular embodiment, the various probes and other reagents such as polymerases, required to permit amplification, RNA signal sequence production, and translation of the coding domain for the reporter gene are provided on a substrate.

In a particular embodiment, the substrate is a paper-based product such as a card and the probes and reagents to facilitate the reactions have been applied to the card in a dried or lyophilised form.

In another embodiment, the substrate comprises plastic, quartz or microfiber.

In another embodiment, the distinct reaction steps are carried out in a microfluidic device.

In another embodiment, some of the reaction components, such as the nucleic acid molecules are bound to a zone in a microfluidic device and the test sample and various reaction reagents are applied to the nucleic acid molecules to initiate a particular reaction (e.g. primer extension using DNA polymerase, transcription using RNA polymerase or translation using cell-free extract). After suitable reaction times the fluids can be washed off and new reaction reagents applied to initiate the next reaction. In this way, a series of reactions can be carried out sequentially. There are known means for binding nucleic acids and then washing away reagents leaving the nucleic acids behind.

In another arrangement, a nucleic acid-containing test sample is used to rehydrate detection components, dried or lyophilised onto plastic/paper or other suitable support medium. The reaction mix is incubated for a suitable period of time and at a suitable temperature (e.g. 30 min at 41° C.). Optionally, the reaction mix is then transferred (e.g. by pipette) to a different site which contains enzymes lyophilised onto plastic/paper or other suitable support medium to facilitate the signal amplification reaction. The reaction is incubated for a suitable period of time and at a suitable temperature (e.g. 2 h at 41° C.) to allow production of the trigger signal RNA. The reaction mix is then transferred to a different site containing lyophilised reporting reagents. The reaction is incubated for a suitable period of time and at a suitable temperature (e.g. 1 h at 41° C.) to allow a visible colour change to be observed if the test sample included the target nucleic acid. This system employs sequential and modular reactions: target detection, signal amplification and signal reporting.

According to another aspect of the invention there is provided a method of detecting a nucleic acid sequence of interest in a sample, the method comprising (a) contacting the sample with first and second probes (two nucleic acid probes) capable of hybridising to the nucleic acid sequence of interest and each other to form a three-way junction (3WJ) complex; (b) generating a single-stranded oligonucleotide sequence from the 3WJ; (c) optionally, using the single-stranded oligonucleotide sequence in step (b) to create multiple copies of a single-stranded oligonucleotide trigger sequence; bringing the oligonucleotide produced in step (b) or (c) into contact with a riboregulator switch sequence which comprises a sequence complementary to the single-stranded oligonucleotide sequence produced in step (b) or (c), a RBS, an initiation codon and a reporter gene, wherein upon binding of the oligonucleotide produced in step (b) or (c) to the riboregulator switch sequence the reporter gene product is produced; and (e) detecting the presence of the reporter gene product, wherein the presence of the reporter gene product indicates that the nucleic acid sequence of interest is in the sample.

According to another aspect of the invention there is provided a trio of nucleic acid sequences for use in a method of detecting a nucleic acid sequence of interest, the first and second sequences are single-stranded oligonucleotides capable of hybridising to a target sequence of interest in an adjacent or substantially adjacent manner and to each other so as to produce a three-way junction complex from which a single-stranded oligonucleotide signal sequence can be produced, and the third sequence is a single or double-stranded sequence that encodes a riboregulator switch sequence containing a hairpin structure and comprising single-stranded and double-stranded domains comprising a single-stranded domain capable of hybridising with some or all of a nucleic acid trigger sequence, a RBS, an initiation codon and a coding domain for a reporter gene arranged such that the ribosome is only able to effect translation when the hairpin loop structure has been disrupted following binding of the nucleic acid trigger sequence to the riboregulator switch sequence.

The riboregulator switch can be provided in double stranded or single stranded form. For example, the riboregulator switch template can be provided in a double stranded DNA (dsDNA) form in a plasmid, as a PCR product or as a synthesised sequence. The riboregulator switch in a single-stranded RNA (ssRNA) form can then be produced from these double-stranded forms using, e.g. an RNA polymerase such as T7 polymerase. Alternatively, the riboregulator switch in a ssRNA form can be provided directly, having produced it by in vitro transcription from a dsDNA plasmid, a PCR product or synthesised gene fragment. If the reporter sequence is not overly long the riboregulator switch could also be synthesised in a ssRNA form. Most suppliers offer to synthesise RNA ultramers of around 150-nt long but the length could be longer if required.

According to another aspect of the invention there is provided a trio of nucleic acid sequences for use in a method of detecting a nucleic acid sequence of interest, the first sequence is a single-stranded oligonucleotide comprising a portion substantially complementary to the sequence of interest and so capable of hybridising thereto, and a portion non-complementary to the sequence of interest but comprising the full-length sequence of a first strand of a double-stranded RNA polymerase promoter and a template signal sequence, the second sequence is a single-stranded oligonucleotide comprising a portion substantially complementary to the sequence of interest and so capable of hybridising thereto, and a portion non-complementary to the sequence of interest but complementary to a part of that portion of the first probe which is non-complementary to the sequence of interest, such that the first and second probes are capable of hybridising to the sequence of interest in an adjacent or substantially adjacent manner, so as to allow complementary portions of the first and second probes to hybridise to each other, and the third sequence is a single-stranded or double-stranded sequence that encodes a riboregulator switch sequence containing a hairpin structure and comprising single-stranded and double-stranded domains comprising a single-stranded domain capable of hybridising with some of an RNA trigger sequence, a RBS, an initiation codon and a coding domain for a reporter gene arranged such that a ribosome is only able to effect translation when the hairpin loop structure has been disrupted following binding of the RNA trigger sequence to the riboregulator switch sequence.

According to another aspect of the invention there is provided a kit for use in detecting the presence in a sample of a nucleic acid sequence of interest, the kit comprising three nucleic acid sequences for use in a method of detecting a nucleic acid sequence of interest, the first and second sequences are single-stranded oligonucleotides capable of hybridising to a target sequence of interest in an adjacent or substantially adjacent manner and to each other so as to produce a three-way junction complex from which a single-stranded oligonucleotide signal sequence can be produced, and the third sequence encodes a riboregulator switch sequence containing a hairpin structure and comprising single-stranded and double-stranded domains comprising a single-stranded domain capable of hybridising with some of a nucleic acid trigger sequence, a RBS, an initiation codon and a coding domain for a reporter gene arranged such that a ribosome is only able to effect translation when the hairpin loop structure has been disrupted following binding of the nucleic acid trigger sequence to the riboregulator switch sequence, optionally one or more reagents and instructions for use.

According to another aspect of the invention there is provided a kit for use in detecting the presence in a sample of a nucleic acid sequence of interest, the kit comprising three nucleic acid sequences for use in a method of detecting a nucleic acid sequence of interest, the first probe sequence is a single-stranded oligonucleotide comprising a portion substantially complementary to the sequence of interest and so capable of hybridising thereto, and a portion non-complementary to the sequence of interest but comprising the full-length sequence of a first strand of a double-stranded RNA promoter and a template signal sequence, the second probe sequence is a single-stranded oligonucleotide comprising a portion substantially complementary to the sequence of interest and so capable of hybridising thereto, and a portion non-complementary to the sequence of interest but complementary to a part of that portion of the first probe which is non-complementary to the sequence of interest, such that the first and second probes are capable of hybridising to the sequence of interest in an adjacent or substantially adjacent manner, so as to allow complementary portions of the first and second probes to hybridise to each other, and the third probe sequence being a single or double-stranded sequence that encodes a riboregulator switch sequence containing a hairpin structure comprising single-stranded and double-stranded domains comprising a single-stranded domain capable of hybridising with some or all of an RNA signal sequence a RBS an initiation codon and a coding domain for a reporter gene arranged such that a ribosome is only able to effect translation when the hairpin loop structure has been disrupted following binding of the RNA signal sequence to the toehold domain, optionally one or more reagents and instructions for use.

According to another aspect of the invention there is provided a solid substrate comprising reagents attached thereon, said reagents comprising: a first probe and a second probe capable of creating a three-way junction with a target sequence of interest and facilitating the generation of an oligonucleotide trigger sequence, and a riboregulator switch sequence probe. In a particular embodiment, the substrate also has one or more of the following attached thereon: a DNA polymerase, an RNA polymerase, ribo-nucleotide triphosphates, deoxyribo-nucleotide triphosphates, a cell-free extract comprising ribosomes and an enzyme substrate reagent.

In a particular embodiment, the solid substrate also comprises an RNA amplification probe. In a particular embodiment, the solid substrate also comprises one or two facilitator probes. In a particular embodiment, the reagents are applied to the substrate in a dried or lyophilised form such that when they are reconstituted by addition of a fluid the reagents can move freely in the fluid.

In one embodiment, the solid substrate is paper-based, such as card.

In one embodiment, the solid substrate is made of plastic, quartz or microfiber.

In another embodiment, the solid substrate is part of a microfluidic device.

In another embodiment, the reagents are attached to one or more zones on the solid substrate.

In one embodiment of the present invention the reagents for the biochemical reactions (detection, amplification and signal reporting) are present on a portable apparatus, such as a paper-based material (e.g. card) or other material (such as plastic, quartz, microfiber etc), optionally in a lyophilised or other dried form, such that when the fluid (e.g. test sample) is applied to the reagents the reactions leading to detection of the target nucleic acid (if present in the sample) are allowed to proceed.

According to another aspect of the invention there is provided a riboregulator switch molecule which comprises a toehold domain, a RBS, an initiation codon and a reporter gene, wherein the molecule is formed from a single-stranded molecule that is capable of self-hybridising to form regions of single and double strands including a single-stranded toehold domain, a partially or fully double-stranded stem domain and a single-stranded hairpin loop domain, wherein the RBS is located in the stem domain and wherein binding of an oligonucleotide signal sequence to the toehold domain and a part or all of a stem domain effects a conformational change in the self-annealed riboregulator switch molecule which allows production of the reporter gene product. In one embodiment the toehold domain is upstream of the RBS. In one embodiment the toehold domain is at the 5′ end of the molecule and is single-stranded. The individual domains and arrangement of these domains in the riboregulator switch sequence molecule is described elsewhere herein.

In particular embodiments, the riboregulator switch is selected from the group consisting of: toehold switch 121, toehold switch 117, toehold switch 119 and toehold switch 42_23. In particular embodiments, the riboregulator switch molecule comprises a sequence selected from the group consisting of SEQ ID NO: 41, 42, 43 and 45, a sequence with at least 90% sequence identity thereto or a sequence with 1, 2, 3, 4, 5 or 6 substitutions therein.

According to another aspect of the invention there is provided a riboregulator switch molecule comprising a sequence selected from the group consisting of SEQ ID NO: 41, 42, 43 and 45, or a sequence with at least 90% sequence identity thereto.

According to another aspect of the invention there is provided a riboregulator switch molecule comprising a sequence selected from the group consisting of SEQ ID NO: 41, 42, 43 and 45, or a sequence with 1, 2, 3, 4, 5 or 6 substitutions therein. By a substitution therein we mean that one of the nucleobases has been substituted by another (e.g. adenine for cytosine).

In particular embodiments, the methods of the invention can utilise any of the sequences disclosed in Table 1.

The present invention improves upon the NASBA-based approaches by enabling the detection of both DNA and RNA sequences rather than only RNA sequences. Further, in the NASBA-based approach, sequences for both the detection/amplification and the reporting systems must be changed for the sensing of different targets, since NASBA amplifies the target nucleic acid sequence. A key differentiating feature, and one that seeks to maximise the commercial potential, of the platform technology of the present invention is that only the probes forming the 3WJ in the detection system need to be changed to detect and report the presence of different nucleic acid sequences. This universal detection approach will allow faster adaptation of the system to detecting different target sequences (e.g. pathogens).

The system of the present invention will have other advantages over the prior art approaches:
A significant advantage of using a 3WJ is the amplification of the oligonucleotide trigger sequence rather than the target sequence. This means that the oligonucleotide trigger sequence generated by the assay does not interfere in the formation of the 3WJ and allows the system to be modular and easily adaptable to different targets. Using pathogen detection as an example, if it is desired to detect more pathogens or different sequences from the same pathogen (or a host's response to that pathogen), only the target-complementary sequence in the probes must be changed, while the probes for signal amplification and the reporting module remain the same.
The assay may be used for detecting either a DNA or an RNA target. RNA is less stable than DNA and persists in an organism for less time. It is therefore a better indication of a pathogen's viability compared to screening for DNA, and so can be a more accurate measure of active infection than DNA. Also, many viruses (e.g. the bovine viral diarrhoea virus, the Newcastle disease virus, etc.) employ RNA as their genetic material. On the other hand, a system able to detect DNA offers more flexibility of uses and can, for instance, facilitate straightforward screening of an organism for genetic biomarkers indicative of increased risk of developing certain cancers. Therefore, an advantage of SMART and 3WJs is that it may detect either DNA or RNA.
The target detection and oligonucleotide trigger amplification can be performed in a single step due to DNA and RNA polymerase being functional under the same conditions. In one embodiment, the present invention integrates the signal reporting step (e.g. a toehold switch with a lacZ system) into the same mixture, making this method even more attractive for one-pot reaction approaches, such as in a microfluidic device or a paper-based diagnostic approach.
Paper-based reactions use low reagent volumes, such that the cost per test could be less than £1 (see e.g. Pardee et al., Cell 165:1255-1266, 2016). This low cost will encourage disposal of the used biosensor and therefore increase biosafety. Furthermore, paper-based devices can be lightweight, providing excellent portability and suitability for field use.
Each biosensor can be designed to respond directly and specifically to the pathogen of concern or to early, invisible, indicators of disease or stress in the host. They will not require culturing of the pathogen or long-term assessment of the host's immune presponse but will instead deliver more accurate and cost-effective solutions than current diagnostic tests on a shorter timescale.
As the present invention is a cell-free system, lacking live genetic material, it will not be subject to regulations relating to the containment of transgenic organisms.

### DETAILED DESCRIPTION OF THE INVENTION

According to one aspect of the invention there is provided a method of detecting a nucleic acid sequence of interest in a sample, the method comprising (a) contacting the sample with first and second probes, capable of creating a three-way junction when the target sequence is present in the sample wherein the first probe comprises a portion substantially complementary to the sequence of interest and so capable of hybridising thereto, and a portion non-complementary to the sequence of interest but comprising a sequence complementary to the second probe and a template signal sequence, and wherein the second probe comprises a portion substantially complementary to the sequence of interest and so capable of hybridising thereto, and a portion non-complementary to the sequence of interest but complementary to a part of that portion of the first probe which is non-complementary to the sequence of interest, such that the first and second probes are capable of hybridising to the sequence of interest in an adjacent or substantially adjacent manner so as to allow complementary portions of the first and second probes to hybridise to each other; (b) causing production of an oligonucleotide sequence that is complementary to the template signal sequence in the first probe; (c) bringing an oligonucleotide trigger sequence into contact with a riboregulator switch sequence, part of which is in the form of a hairpin loop structure, comprising an RNA sequence having single-stranded and double-stranded domains comprising a single-stranded domain capable of hybridising with part of the oligonucleotide trigger sequence, a RBS, an initiation codon and a coding domain for a reporter gene arranged such that the ribosome is only able to effect translation when the hairpin loop structure has been disrupted following binding of the oligonucleotide trigger sequence to the riboregulator switch; and (d) detecting the reporter gene product; wherein the presence of the reporter gene product indicates the presence of the sequence of interest in the sample; and, wherein the oligonucleotide trigger sequence is either the oligonucleotide sequence produced in step (b) or an oligonucleotide sequence produced using the oligonucleotide sequence produced in step (b) as an intermediate in an amplification reaction.

The template signal sequence is a sequence that is the complement of an oligonucleotide that is used as a signal sequence, e.g. oligonucleotide trigger.

The oligonucleotide produced in step (b) acts as a primer for amplification of an oligonucleotide trigger sequence molecule capable of activation of a riboregulator switch, or can itself trigger activation of a riboregulator switch.

The oligonucleotide trigger sequence can be RNA or DNA.

In one embodiment the oligonucleotide trigger sequence comprises or consists of the sequence disclosed in SEQ ID NO: 35 or 38.

In one embodiment, the oligonucleotide sequence in step (b) is produced directly or indirectly by primer extension of probe 2 using probe 1 as template and a DNA polymerase. For example, if primer extension of probe 2 using probe 1 as template creates a restriction enzyme site which when nicked with the appropriate restriction enzyme releases a single-stranded molecule that can serve as the oligonucleotide trigger sequence, said molecule can be considered to have been produced directly from the primer extension. If the oligonucleotide sequence produced in step (b) arises following transcription using RNA polymerase on the formed double-stranded RNA promoter, the RNA signal sequence can be considered to have been produced indirectly from the primer extension. Alternatively, if the single-stranded oligonucleotide produced from the primer extension is subjected to a separate amplification reaction to produce a distinct single-stranded oligonucleotide trigger sequence then said molecule can also be considered to have been produced indirectly from the primer extension of probe 2 using probe 1 as template.

In one embodiment, the riboregulator switch sequence comprises a fully or partially double-stranded stem domain, a loop domain and distinct single-stranded domains.

Steps (a) and (b) are concerned with detecting the presence of a target sequence of interest, wherein if said sequence is present a single-stranded oligonucleotide (DNA or RNA) molecule is generated from the 3WJ formed when the two probes hybridise with the target sequence. Optionally, this oligonucleotide molecule can be amplified to create many copies of the same oligonucleotide or a different oligonucleotide. Optionally the different (second) oligonucleotide may comprise the sequence of the first oligonucleotide. For example, the second oligonucleotide may be the same as the first oligonucleotide except for the addition of extra bases at one or both ends.

Thus, in one embodiment, the oligonucleotide trigger sequence is the oligonucleotide sequence generated and released from the 3WJ produced without an additional amplification step. In another embodiment, the oligonucleotide trigger sequence has the same sequence as the oligonucleotide sequence generated and released from the 3WJ, but it has been produced (in part) by an amplification step. In another embodiment, the oligonucleotide trigger sequence has a different sequence to that of the oligonucleotide sequence generated and released from the 3WJ. In another embodiment, the oligonucleotide trigger sequence has a sequence that comprises some or all of the oligonucleotide sequence generated and released from the 3WJ, for example the oligonucleotide trigger sequence is the same as the oligonucleotide sequence produced from the 3WJ but it has additional nucleotides at one end. A distinct oligonucleotide sequence molecule can be produced if the amplification probe that the original oligonucleotide sequence hybridises to encodes a distinct oligonucleotide sequence at its 5′-end, such that following primer extension of the bound oligonucleotide sequence a functional double-stranded RNA promoter and an RNA signal sequence is generated. RNA polymerase can then bind to the promoter and generate the new RNA signal sequence molecule. It is this molecule that can then serve as the oligonucleotide trigger sequence in the subsequent detection.

In one embodiment the amplification probe comprises or consists of the sequence disclosed in SEQ ID NO: 3, 39 or 40.

In a particular embodiment, prior to contacting with the riboregulator switch the oligonucleotide trigger sequence is amplified by contacting the oligonucleotide generated and released from the 3WJ with a single-stranded nucleic acid amplification sequence comprising three regions, a first region comprising a sequence sufficiently complementary to the oligonucleotide sequence produced from the 3WJ to allow hybridisation thereto, a second region encoding the full-length sequence of a first strand of a double-stranded RNA promoter and a third region comprising a first strand of a double-stranded RNA signal sequence, such that extension of the bound oligonucleotide sequence with a nucleic acid polymerase, using the nucleic acid amplification sequence as a template produces a functional RNA polymerase promoter and double-stranded signal sequence which can then be used by RNA polymerase to produce an RNA molecule which serves as the oligonucleotide trigger sequence. In one embodiment, the second and third region sequences can be the same as that portion of the first probe that is non-complementary to the sequence of interest. In one embodiment, the nucleic acid amplification sequence is present at the same time as the sample is contacted with the first and second probes. In one embodiment, the oligonucleotide sequence produced from the 3WJ is an RNA signal sequence which is an intermediate oligonucleotide sequence which following binding to the amplification probe effects production of a different oligonucleotide sequence. This “different” oligonucleotide sequence may contain a sequence that is identical to the oligonucleotide sequence produced from the 3WJ and is capable of hybridising to the riboregulator switch molecule. In this way, both the original oligonucleotide sequence produced from the 3WJ and the second oligonucleotide sequence produced after the amplification reaction are capable of hybridising to the riboregulator switch and activating it.

Region 1 is at the 3′ end of the amplification probe and region 3 is at the 5′ end of the probe.

In one embodiment, the oligonucleotide sequence produced in step (b) is a DNA molecule. In one embodiment, the oligonucleotide sequence produced in step (b) is an RNA molecule. In one embodiment, the oligonucleotide trigger sequence is a DNA molecule. In one embodiment, the oligonucleotide trigger sequence is an RNA molecule. In one embodiment, both the oligonucleotide sequence produced in step (b) and the oligonucleotide trigger sequence are DNA molecules. In one embodiment, both the oligonucleotide sequence produced in step (b) and the oligonucleotide trigger sequence are RNA molecules. In one embodiment, the oligonucleotide sequence produced in step (b) is a DNA molecule and the oligonucleotide trigger sequence is an RNA molecule. In one embodiment, the oligonucleotide sequence produced in step (b) is an RNA molecule and the oligonucleotide trigger sequence is a DNA molecule.

In one embodiment, the oligonucleotide trigger sequence has the same sequence as the oligonucleotide sequence produced in step (b). In one embodiment, the oligonucleotide trigger sequence comprises the sequence of the oligonucleotide sequence produced in step (b).

In one embodiment the oligonucleotide trigger sequence comprises or consists of the sequence disclosed in SEQ ID NO:35 or 38.

In one embodiment, the oligonucleotide trigger sequence is a sequence produced when the oligonucleotide sequence produced in step (b) is subjected to an amplification reaction. In one embodiment, the amplification reaction involves contacting the oligonucleotide sequence produced in step (b) with an amplification probe comprising three regions, a first region comprising a sequence sufficiently complementary to the oligonucleotide sequence produced in step (b) to allow hybridisation thereto, a second region encoding the full-length sequence of a first strand of a double-stranded RNA promoter and a third region comprising a first strand of a double-stranded nucleic acid signal sequence, such that extension of the bound oligonucleotide sequence produced in step (b) with a nucleic acid polymerase using the nucleic acid amplification sequence as a template, produces a functional RNA polymerase promoter and double-stranded signal sequence which can then be used by RNA polymerase to produce the oligonucleotide trigger sequence. In this particular arrangement, the oligonucleotide trigger sequence is a single-stranded RNA molecule.

In one embodiment, the riboregulator switch sequence comprises a toehold domain that part of the oligonucleotide trigger sequence can hybridise to. In another embodiment, part of the oligonucleotide trigger sequence hybridises to the hairpin loop of the riboregulator switch molecule. In another embodiment, part of the oligonucleotide trigger sequence hybridises to the double-stranded stem region of the riboregulator switch.

There are many ways that a single-stranded oligonucleotide sequence can be produced from the 3WJ that is formed when the two probes hybridise to that target sequence of interest. In one embodiment, one of the probes comprises a restriction enzyme recognition sequence, such as one that is recognised by an enzyme that nicks one strand only. In one embodiment, the first probe comprises a restriction enzyme recognition sequence. In one embodiment, the first probe comprises a Nb.Bsml restriction enzyme recognition sequence. In one embodiment, the first probe comprises a restriction enzyme recognition sequence, such as one for Nb.Bsml, and the oligonucleotide sequence produced in step (b) is created by primer extension of the second probe with a nucleic acid polymerase using the first probe as template, allowing the double-stranded primer extension product to be nicked by a restriction enzyme that recognises the restriction enzyme recognition sequence generated by the primer extension and allowing the oligonucleotide to separate from its complement strand. In one embodiment, the oligonucleotide sequence produced in step (b) is amplified by rolling-circle amplification, such as primer-generation rolling circle amplification (PG-RCA) or linear rolling circle amplification (LRCA).

PG-RCA can be carried out at constant temperature by mixing a circular DNA probe, DNA polymerase, and a nicking enzyme. The circular probes comprise a hybridization sequence complementary to the signal oligonucleotide and a complementary nicking site. PG-RCA initiates from hybridization of a circular probe to the signal oligonucleotide. A nicking enzyme recognizes the duplex structure and cleaves the sample DNA at the nicking site, which triggers a cascade reaction of linear rolling circle amplification (LRCA) and nicking reactions. LRCA produces long, concatenated copies of the circular probe sequence, while the nicking reaction generates multiple “primers” for the circular probe from the LRCA product. Accordingly, these reactions continuously initiate each other. Also, as multiple reaction cycles can be initiated from a single cycle, the PG-RCA reaction accumulates LRCA products and “primers” in an exponential manner over time. Conveniently, PG-RCA can be carried out using Vent (exo-) DNA polymerase and a thermostable strand specific nicking enzyme such as Nb.Bsml (see Murakami et al. Nucleic Acids Res. 37:e19, 2009)

Nb.Bsml is a nicking endonuclease that cleaves only one strand of a double-stranded DNA substrate and is most active at 65° C. It recognises the sequence 5′-GAATGCN-3′. Nb.Bsml (NEB #R0706) is a bottom-strand specific variant of Bsml (NEB #R0134) discovered from a library of random mutants. Other nicking enzymes such as Nt.BspQl, Nt.CviPll and Nt.Alwl could also be used.

Another way to produce a single-stranded oligonucleotide from the formed 3WJ is to generate an RNA promoter sequence that an RNA polymerase can use to produce a single-stranded (oligonucleotide) RNA transcript. A functional double-stranded RNA promoter sequence can either be formed when the 3WJ assembles or it can be generated by primer extension of one probe using the other as template, this extension can then form the double-stranded RNA promoter. In one embodiment, one of the probes comprises the full-length sequence of a first strand of a double-stranded RNA promoter and a signal sequence. In one embodiment, the first probe comprises the full-length sequence of a first strand of a double-stranded RNA promoter and a signal sequence. In one embodiment, the second probe comprises part of an RNA polymerase promoter sequence, which part is capable of hybridising to the first probe. In one embodiment, part of an RNA polymerase promoter sequence is provided by the target sequence. In one embodiment, assembly of the 3WJ complex creates a substantially functional RNA polymerase promoter. WO9937805 describes how to assemble 3WJ complexes that create a substantially functional RNA polymerase promoter.

According to another aspect of the invention there is provided a method of detecting a nucleic acid sequence of interest in a sample, the method comprising (a) contacting the sample with first and second probes, wherein the first probe comprises a portion substantially complementary to the sequence of interest and so capable of hybridising thereto, and a portion non-complementary to the sequence of interest but comprising the full length sequence of a first strand of a double-stranded RNA promoter and a template signal sequence, and wherein the second probe comprises a portion substantially complementary to the sequence of interest and so capable of hybridising thereto, and a portion non-complementary to the sequence of interest but complementary to a part of that portion of the first probe which is non-complementary to the sequence of interest, such that the first and second probes are capable of hybridising to the sequence of interest in an adjacent or substantially adjacent manner, so as to allow complementary portions of the first and second probes to hybridise to each other; (b) causing extension of the second probe with a nucleic acid polymerase, using the first probe as a template so as to produce a functional RNA polymerase promoter and double-stranded signal sequence; (c) causing production of an RNA signal sequence when the double-stranded functional RNA polymerase promoter and signal sequence produced in (b) is contacted with an RNA polymerase; (d) bringing an oligonucleotide trigger sequence into contact with a riboregulator switch sequence, part of which is in the form of a hairpin loop structure comprising an RNA sequence having single-stranded and double-stranded domains comprising a single-stranded domain capable of hybridising to part of the oligonucleotide trigger sequence, a RBS, an initiation codon and a coding domain for a reporter gene arranged such that a ribosome is only able to effect translation when the hairpin loop structure has been disrupted following binding of the oligonucleotide trigger sequence to the riboregulator switch; and (e) detecting the reporter gene product; wherein the presence of the reporter gene product indicates the presence of the sequence of interest in the sample; and, wherein the nucleic acid trigger sequence is either the signal sequence produced in step (c) or is an oligonucleotide sequence produced when the signal sequence produced in step (c) is hybridised to an amplification probe capable of generating the oligonucleotide trigger sequence. In a particular embodiment, the RBS is not accessible to the ribosome when the hairpin loop structure is present. In another embodiment, although the ribosome can bind the RBS it cannot effect translation due to the structure of the riboregulator switch, i.e. translation cannot proceed when the hairpin loop structure is present. The RBS only becomes available for ribosome binding and translation when the hairpin loop structure has been partially or fully disrupted following binding of the oligonucleotide trigger sequence to the riboregulator switch sequence.

As used herein, the term “probe” means a nucleic acid sequence, typically a single-stranded oligonucleotide that has regions complementary to and thus capable of hybridising to another target sequence. The probe/nucleic acid sequence can be any nucleic acid type, including RNA, DNA, PNA or LNA. In one embodiment, the first and second probes comprise DNA, RNA, PNA (peptide nucleic acid), LNA (locked nucleic acid) or any combination thereof. The two sequences that form the 3WJ with the target sequence, and the riboregulator switch sequence (also referred to herein as probe 3) are referred to herein as probes.

The first and second probes may comprise any nucleic acid, such as DNA, RNA, PNA (peptide nucleic acid) or LNA (locked nucleic acid), or any combination thereof. In one embodiment, the first and second probes are composed of DNA. In certain situations, probes comprising PNA or LNA may have greater thermal stability when bound to DNA and so may be more specific for target DNA sequences. As such, the regions of the first and second probes that hybridise with the target sequence of interest may comprise PNA or LNA to enhance the specificity of binding. The portions of the probes that do not anneal to the target sequence of interest but are designed to anneal to each other could also be of PNA or LNA or could be DNA. Thus, hybrid probes comprising, for example, DNA and PNA or DNA and LNA are envisaged. However, as PNA is not recognised by any polymerase, it is important that it is only used in portions of the probe that is not meant to function as a template and thus do not require copying.

The first probe comprises a sequence that is substantially complementary to the target sequence of interest and capable of hybridising thereto. Typically, the sequence substantially complementary to the target sequence will contain 8-50 nucleotides, such as 8-25, 10-25, 15-25 and 20-30 nucleotides. The portion that is not complementary to the target sequence comprises a region of nucleotides that are capable of hybridising to part of the second probe; this region may comprise any number of nucleotides but typically from about 4-12, such as 6-8 nucleotides. It may have a portion that encodes the full-length sequence of an RNA polymerase promoter. Part of this sequence could be in the zone that hybridises to the second probe. Alternatively, none of the sequence coding for the RNA polymerase promoter is in the region that hybridises to the second probe. Any RNA polymerase promoter sequence can be used such as T3, T7, SP6 or strong phage-derived PN25 constitutive E coli promoter (PN25), or any mutant forms thereof which are known to those skilled in the art. The first probe may have a portion that encodes a restriction enzyme recognition site.

Examples of suitable RNA polymerase promoter sequences include: T7 RNA polymerase promoter sequence: TAATACGACTCACTATA[GGG]—SEQ ID NO: 11; T3 RNA polymerase promoter sequence: AATTAACCCTCACTAAA[GGG]—SEQ ID NO: 12; SP6 RNA polymerase promoter sequence: AATTTAGGTGACACTATAGAA—SEQ ID NO: 13; and PN25 promoter sequence:

TCATAAAAAATTTATTTGCTTTCAGGAAAATTTTTCTGTATAATAGATTCATAAATTT—SEQ ID NO: 14.

The region [GGG] is leader sequence (+1 to +3 nt transcribed) that encourages efficient transcription by the T7 or T3 RNA polymerase.

In particular embodiments, formation of the 3WJ or extension of the second probe results in formation of an RNA polymerase promoter, such as T3, T7 or SP6 RNA polymerase promoter, which allows for transcription of multiple RNA signal sequence copies of a sequence complementary to part of the first probe.

In particular embodiments, formation of the 3WJ or extension of the second probe results in formation of a T7 promoter.

In another embodiment, extension of the second probe results in formation of a T7 promoter and the complement of the signal sequence in probe 1.

In one embodiment, the restriction enzyme recognition site in the first probe is recognised by a restriction enzyme that nicks one strand of a double-stranded molecule. In one embodiment, the enzyme is Nb.Bsml. In one embodiment, primer extension of the second probe results in formation of the double-stranded restriction enzyme recognition sequence.

In one embodiment, the nucleic acid polymerase in step (b) is a thermophilic DNA polymerase.

In particular embodiments, the nucleic acid polymerase in step (b) is selected from: Bacillus stearothermophilus (Bst) DNA polymerase, Vent(exo-) DNA polymerase, a derivative of 9° N™ DNA Polymerase such as Therminator DNA polymerase (M0261, New England Biolabs), and Klenow polymerase. There are various mutant or variant forms of these polymerases (e.g. Bst 2.0 and Bst 3.0), and any suitable mutant or variant form could be utilised. In one embodiment, the nucleic acid polymerase that extends the second probe is Bacillus stearothermophilus (Bst) DNA polymerase. This polymerase is suitable for isothermic reactions, allowing the reaction to proceed at a single temperature, such as room temperature, 41° C. or 55° C., without needing thermal cycling.

In embodiments that involve the use of a substantially functional RNA promoter, adjacent to the RNA promoter sequence there is nucleotide sequence that encodes an RNA signal sequence arranged such that a single-stranded RNA sequence complementary to the sequence in probe 1 can be created by the RNA polymerase that binds to the promoter. The RNA signal sequence is typically from 10-80 nucleotides in length. Smaller sequences of 10-30 nucleotides work well. If it is desired to quantitate the amount of sequence produced (e.g. by qPCR) longer sequences, such as 50-70nt are convenient. By substantially functional is meant that some amount of transcription of the sequence following the promoter occurs, i.e. that an oligonucleotide RNA signal sequence is produced which can then be used to bind and trigger the riboregulator switch sequence or used in an amplification reaction to generate an oligonucleotide trigger sequence in a suitably amplified amount.

The efficiency of initiation of RNA synthesis by the RNA polymerase promoter is affected by sequences adjacent and downstream to the promoter. In particular, a region of twelve bases (the “+12 region”) is required for optimum RNA transcription (see e.g. WO1999037806). The person skilled in the art is able to design optimal sequences for efficient transcription using a particular RNA polymerase. Suitable, +1 to +12 sequences (in 5′ to 3′ direction) for T7 polymerase include: ATCGTCAGTCCC (SEQ ID NO: 15), GCTCTCTCTCCC (SEQ ID NO: 16), ATCCTCTCTCCC (SEQ ID NO: 17) and GTTCTCTCTCCC (SEQ ID NO: 18).

Ideally the 3′ terminus of the first probe is blocked to prevent chain extension. It will be apparent to those skilled in the art how this can be achieved, e.g. using a 3′ dideoxynucleotide (e.g.3′ddC), 3′ inverted dT, 3′ C3 spacer, 3′ amino (e.g. 3′-propyl) or 3′ phosphorylation (e.g. 3′ phosphate).

For efficient RNA synthesis, it may also be desirable to block the 5′ end of the first probe. At the end of the sequence encoding the RNA signal sequence there may be an RNA polymerase termination signal to cause termination of transcription. For example, positioning the sequence AACAGA in 5′ end of the template probe (probe 1). He et al., (J. Biol. Chem. 273:18802-18811, 1998) disclose that the sequence AACAGA is particularly efficient at terminating T7 polymerase-mediated transcription. Thus, in one embodiment near or at the 5′ end of probe 1 there is the sequence AACAGA.

The second probe comprises a portion substantially complementary to the target sequence of interest and a portion non-complementary to the sequence of interest but complementary to a part of that portion of the first probe which is non-complementary to the sequence of interest.

The portion substantially complementary to the target sequence of interest is capable of hybridising thereto in a position adjacent or substantially adjacent to the first probe such that complementary portions of the first and second probes can hybridise to each other. Thus, when the first and second probes bind to the target sequence of interest and to each other a three-way junction (3WJ) structure is formed (see FIGS. 2(a) and 3(a)). It is important that the first and second probes, when bound to the target sequences, are adjacent or substantially adjacent to each other so as to allow the complementary regions of the first and second probes to anneal to each other. In certain situations, the first and second probes are hybridised to the target nucleic acid sequence such that there are no gaps/nucleotides of the target sequence left without base-pairing to the complementary sequences of the first and second probes. In this scenario, the first and second probes are herein referred to as being adjacent to each other on the target sequence. As used herein, the first and second probes are said to be substantially adjacent on the target sequence when there is one or more, up to 5, nucleotides on the target sequence between the portions on the target sequence that are base-paired with the first and second probes. It will be appreciated by the person skilled in the art that the proximity of probes 1 and 2 to each other allows the target non-complementary sequences of the probes to base-pair with themselves, and thus the greater the distance between the location of binding of the first probe and the second probe on the target sequence the greater the amount of unpaired sequence there may be, and thus the greater likelihood of instability in binding. Nevertheless, the person skilled in the art can design probes that are not adjacent to each other yet are still capable of binding the target sequence and to those parts of each other so as to form the 3WJ.

With regard to the second probe, the sequence substantially complementary to the target sequence will typically contain 8-50 nucleotides, such as 8-25, 10-25, 15-25 nucleotides. The portion that is non-complementary to the target sequence comprises a region or regions of nucleotides that are capable of hybridising to part of the first probe; this region may comprise any number of nucleotides but typically from about 2-15 nucleotides. Part, but not all, of this sequence may encode part of the RNA polymerase promoter sequence. In one embodiment, when the target sequence and the first and second probe sequences have hybridised together to form the 3WJ a functional double-stranded RNA polymerase promoter sequence has been created. In another embodiment, after the 3WJ has been formed no functional RNA promoter sequence has been formed. If the second probe is to be extended in a primer extension reaction, there is a free —OH group at the 3′ end of the second probe from which primer extension can proceed. In operation, primer extension using the first probe as template can create a functional double-stranded sequence that includes a restriction enzyme recognition sequence or a double-stranded RNA promoter and double-stranded RNA signal sequence. If a functional double-stranded RNA promoter sequence is formed, then in the presence of RNA polymerase, the RNA polymerase can bind to the double-stranded promoter with the subsequent transcription of an RNA signal sequence.

In a particular embodiment, in addition to the first and second probes one or more additional probes termed facilitator probes (FPs) capable of hybridising to the target sequence either side of the regions that probe 1 and 2 hybridise to can be included. (e.g. see FIGS. 2(a), 3(a) and 4(a)). Such facilitator probes serve to stabilise the 3WJ. In one embodiment, two facilitator probes are included for use in the methods of the invention. The presence and use of FPs is not essential to the working of the invention.

The FPs can be single-stranded oligonucleotides of DNA, PNA or LNA. Typically, they will be between 8 and 40 nucleotides in length. Conveniently 10-25 nucleotides in length. They are designed to hybridise to the target sequence either side of the region that the first and second probes hybridise to. Their purpose is to inhibit (under the hybridisation conditions employed) the target sequence strand from annealing with its complementary strand to make the target sequence of interest accessible to the first and second probes. The FPs can hybridise to the target strand in a position adjacent to the first and second probes or may hybridise to the target strand at a distance, such as 5-80 nucleotides away. It will be appreciated that the facilitator probe located beside the first probe and the facilitator probe beside the second probe need not be the same length, composition (PNA, LNA, DNA etc.) or distance from the first or second probe. Such that one can be adjacent the first probe but the other can be 50 nucleotides from the second probe. One could be 10 nucleotides in length the other 20. In general, facilitator probes are not necessary when the target nucleic acid is single-stranded.

In one embodiment, two further probes hereinafter referred to facilitator probe 1 (FP1) and facilitator probe 2 (FP2) are used in step (a), wherein FP1 comprises a sequence capable of hybridising to the target sequence of interest at a site adjacent or substantially adjacent to the annealing site of the first probe and FP2 comprises a sequence capable of hybridising to the target sequence of interest at a site adjacent to or substantially adjacent to the annealing site of the second probe, wherein FP1 and FP2 serve to stabilise the 3WJ complex.

In one embodiment, the facilitator probes are single-stranded oligonucleotides of DNA, PNA or LNA, or any combination thereof. In another embodiment, the FPs are between 8 and 40 nucleotides in length. In another embodiment, the FPs are10-25 nucleotides in length.

WO 99/037806 (Cytocell Ltd.) teaches the incorporation of one or more destabilising moieties into the first and/or second probe to prevent these probes hybridising in the absence of hybridisation to the target sequence. These types of destabilising moieties can be incorporated into the first and/or second probes of the present invention.

A destabilising moiety may be present on the first probe or the second probe and is typically present in the region that is not complementary to the target sequence of interest, often within 3-5 nucleotides of the sequence that is hybridised to the target sequence of interest. The destabilising moiety(ies) serve to inhibit the first and second probes from annealing in the absence of annealing to the target sequence of interest, and may also assist access of the polymerase. U.S. patent Ser. No. 6392,593, lists various destabilising moieties that can be used in the present invention, including, for example: Hexaethylene glycol (Hex), pentamethylene, hexamethylene, inosine, propyl, nitropyrrole or combinations thereof, or propyl-Hex-propyl, propyl-Hex-Hex-propyl, or butyl-Hex-butyl etc.

In one embodiment, one or more destabilising moieties are present in the first and/or second probe.

In particular embodiments, the destabilising moiety comprises hexaethylene glycol (Hex), pentamethylene or hexamethylene.

The person skilled in the art would be able to select and include one or more destabilising moieties into either or both of the first and second probes. In view of the size of the destabilising moiety, the number of bases opposite said moiety will need to be determined and included in the companion probe to ensure that the complementary sequences of the first and second probes are able to anneal together; thus, one destabilising moiety on one probe may require there to be 3-4 nucleotides on the other probe. These nucleotides are effectively spacer nucleotides until the two probes can anneal again. Hex is one particularly suitable destabilising moiety, and it can be present singly or in tandem such as 5 times. Because the destabilising moiety cannot base pair with nucleotides on the other strand a bulge structure exists which destabilises and inhibits hybridisation of the first and second probes to each other unless the target sequence is present.

In a particular embodiment, the first and/or second probe comprises one or more destabilising moieties which cannot base pair with the reciprocal member of the pair of probes, thereby preventing hybridisation of the first and second probes in the absence of the target sequence of interest. This minimises the amount of false positive RNA signal sequence generated yielding more specificity. In another embodiment, the destabilising moiety is covalently linked to the first or second probe. In another embodiment, the first and/or second probe comprises a destabilising moiety which cannot base pair with the reciprocal probe, thereby preventing hybridization of the first and second probes in the absence of the sequence of interest. In another embodiment, the destabilising moiety does not comprise nucleic acid base. In another embodiment, the destabilising moiety is selected from: Hexaethylene glycol (Hex), pentamethylene or hexamethylene, inosine, propyl, nitropyrrole, ribose or combinations thereof, or propyl-Hex-propyl, propyl-Hex-Hex-propyl. In one embodiment, a destabilising moiety is present in the first probe. In another embodiment, a destabilising moiety is present in the second probe.

The hybridisation of the first and second probes to the target sequence of interest in a test sample forms a three-way junction structure. If the second probe is to be extended, DNA polymerase provided in the reaction mixture extends the second probe using the first probe as template to synthesise double-stranded nucleic acid. In one embodiment, the double-stranded nucleic acid molecule includes a restriction enzyme recognition sequence. In another embodiment, the double-stranded nucleic acid sequence includes a functional RNA polymerase (e.g. T7 RNA polymerase) and a double-stranded template encoding an RNA signal sequence. RNA polymerase (e.g. T7), added to or already present in the reaction mixture can then bind to the newly created double-stranded RNA polymerase promoter and transcribes the template to create multiple single-stranded RNA signal sequence molecules. Optionally, these RNA signal sequence molecules can be amplified using the nucleic acid amplification probe and appropriate polymerases. Thus, if the target sequence of interest is present in the test sample multiple copies of the same oligonucleotide sequence is produced. The presence of these can then be detected using a riboregulator switch (e.g. including those containing a toehold domain), as further described below, that can generate a measurable signal, such as by producing an enzyme (like lacZ) that is capable of effecting a colour change reaction using the appropriate substrate.

RNA Signal Amplification

When the target sequence is present in a test sample, step (a) produces a three-way junction (3WJ) between the target sequence, the first probe and the second probe.

In one embodiment, formation of the 3WJ creates a substantially functional RNA promoter sequence which an RNA polymerase can use to transcribe an RNA oligonucleotide sequence using the first probe as template. This transcription can occur from single-stranded template, or double-stranded template that may have been synthesised by primer extension of the second probe using the first probe as template. Thus, if the first probe comprises the full-length sequence of a first strand of a double-stranded promoter, the target sequence comprises a part of a second strand of the double-stranded promoter which is complementary to a part of the first strand, and the second probe comprises the rest of the second strand of the double-stranded promoter which is complementary to a part of the first strand, such that a functional promoter is formed when the first probe is hybridised to both the target sequence and to the second probe. Such an arrangement is taught in WO9937805. There is no extension required for promoter formation.

An example of the formation of a functional double-stranded RNA promoter is when the first three (5′) bases of the promoter sequence is complemented by three bases (e.g. 3′ ATT 5′) in the target sequence. Formation of the 3WJ creates a functional double-stranded RNA promoter.

In one embodiment, the first probe comprises a sequence for an RNA promoter and the complement of an RNA signal sequence. Primer extension of the second probe using the first probe as template generates a double-stranded RNA promoter and RNA signal sequence, RNA polymerase can then bind the RNA promoter and transcribe the RNA signal sequence. In this way, multiple copies of the RNA signal sequence are produced. Optionally, the number of RNA signal sequence molecules produced can be amplified by contacting the RNA signal sequence molecules with an amplification probe such that the RNA signal sequence and amplification probe hybridise in such a way that primer extension can proceed from the 3′ end of the signal sequence using the amplification probe as a template to produce a functional double-stranded RNA promoter sequence and RNA signal sequence. RNA polymerase can then bind the double-stranded promoter and produce the RNA signal sequence molecules encoded within the 5′ end of the amplification probe. Through appropriate design of the amplification probe multiple copies of the same RNA signal sequence that was the primer sequence for the amplification probe or a second distinct RNA signal sequence can be generated. In a particular embodiment, the most abundantly produced RNA signal sequence molecules serve as the trigger oligonucleotide sequence for the subsequent riboregulator switch detection and signal generation. Equally, in embodiments that utilise a restriction enzyme recognition sequence to nick the double-stranded extension product liberating one of the oligonucleotide strands, said oligonucleotide strand could then be contacted with an amplification probe, primer extended to create a functional RNA promoter and RNA signal sequence so as to allow the production via transcription of multiple copies of a single-stranded RNA sequence. These RNA molecules can then serve as the oligonucleotide trigger sequence molecules. As noted above, in one embodiment, the method can be employed by detection of the original oligonucleotide sequence produced from the 3WJ. However, in order to generate a stronger or faster detectable signal it is possible to amplify the number of RNA trigger sequence molecules produced. Thus, in one embodiment, the oligonucleotide trigger sequence is the oligonucleotide sequence generated from the 3WJ produced without an additional amplification step. In another embodiment, the oligonucleotide trigger sequence has the same sequence as the oligonucleotide sequence generated from the 3WJ, but it has been produced (in part) by an amplification step. In another embodiment, the oligonucleotide trigger sequence has a different sequence to that of the oligonucleotide sequence generated from the 3WJ. In another embodiment, the oligonucleotide trigger sequence has a sequence that comprises some or all of the oligonucleotide sequence generated from the 3WJ, for example the oligonucleotide trigger sequence is the same as the oligonucleotide signal sequence produced from the 3WJ but it has additional nucleotides at one end. A distinct oligonucleotide sequence molecule can be produced if the amplification probe that the original oligonucleotide sequence hybridises to encodes a distinct RNA sequence at its 5′ end, such that following primer extension of the bound oligonucleotide sequence a functional double-stranded RNA polymerase promoter and distinct RNA signal sequence is generated. RNA polymerase can then bind to the promoter and generate the new RNA signal sequence molecule. It is this molecule that can then serve as the oligonucleotide trigger sequence in the subsequent detection step.

In a particular embodiment, following the generation of the oligonucleotide from the 3WJ, said oligonucleotide sequence is amplified by contacting the oligonucleotide produced with a single-stranded nucleic acid amplification sequence comprising three regions, a first region comprising a sequence sufficiently complementary to the oligonucleotide sequence produced from the 3WJ to allow hybridisation thereto, a second region encoding the full-length sequence of a first strand of a double-stranded RNA promoter and a third region comprising a first strand of a double-stranded RNA signal sequence, such that extension of the bound oligonucleotide sequence with a nucleic acid polymerase, using the nucleic acid amplification sequence as a template produces a functional RNA polymerase promoter and double-stranded signal sequence which can then be used by RNA polymerase to produce the RNA signal sequence. In one embodiment, the RNA polymerase promoter sequence on the amplification probe can be the same as that on the first probe. In another embodiment, the RNA signal sequence on the amplification probe can be the same as that on the first probe. In another embodiment, the RNA signal sequence on the amplification probe is different to that on the first probe. In another embodiment, the RNA signal sequence on the amplification probe comprises the sequence of the RNA signal sequence on the first probe. In another embodiment, the second and third region sequences can be the same as that portion of the first probe that is non-complementary to the sequence of interest. In one embodiment, the nucleic acid amplification sequence is present at the same time as the sample is contacted with the first and second probes. In one embodiment, the oligonucleotide sequence produced from the 3WJ is an intermediate sequence capable of binding to the amplification probe to effect production of an RNA signal sequence. This “new” RNA signal sequence may contain a sequence that is identical to the oligonucleotide sequence produced from step (c) and this identical sequence could be capable of hybridising to the riboregulator switch molecule. In this way, both the original oligonucleotide sequence and the second RNA signal sequence produced after the amplification reaction are capable of hybridising to the riboregulator switch and activating it. This system works particularly well when the original oligonucleotide produced from the 3WJ is an RNA molecule.

With regard to the amplification probe, the first region is at the 3′ end of the amplification probe and the third region is at the 5′ end of the probe.

The signal sequence region within the first probe is a sequence used for generating an RNA transcript or is an oligonucleotide that can be released by a restriction enzyme.

In a particular embodiment, the RNA signal sequence produced when RNA polymerase binds to the double-stranded RNA polymerase promoter sequence generated by the first and second probes (with or without primer extension from probe 2) and transcribed therefrom, is subject to amplification by contacting with a nucleic-acid amplification probe resulting in a chain reaction of RNA signal sequence amplification.

The 3′ end of a de novo produced RNA transcript (RNA signal sequence) produced from the template portion of the first probe can be hybridised to the amplification probe and extended by a polymerase and reagents in the reaction mixture. This creates a functional double-stranded RNA promoter which is recognised by the appropriate RNA polymerase which then generates more RNA signal sequence transcripts (the complement of the sequence at the 5′ end of the DNA amplification probe/oligonucleotide). In turn, if the 3′ region of these newly produced transcripts can hybridise to the amplification probe further rounds of extension and transcription can proceed.

Desirably, the amplification step is accomplished by performing two or more nucleic acid synthesis steps in a cyclical manner, such that the nucleic acid product of a first synthesis step acts as the primer for a second nucleic acid synthesis step, the product of which acts as the primer for the first nucleic acid synthesis step, and so on. Cycling amplification of this sort is disclosed in WO93/06240.

Alternatively, the RNA signal sequence may be the subject of “stepped” amplification such as disclosed in WO 2001/009376.

In one embodiment, the RNA transcripts produced from the amplification probe comprise sequences which are identical to those present in an RNA transcript produced originally from the 3WJ, such that a cycle is formed allowing massive amplification of the original transcript thereby greatly enhancing the sensitivity of the detection method of the invention.

If the RNA transcript produced from the amplification reaction is the same as that produced from the 3WJ, the ‘amplification probe’ will have the same sequence upstream and downstream of the promoter. In order to get the same RNA produced, RNA amplification probe will have to contain exactly the same sequence upstream and downstream T7 promoter. If the oligonucleotide signal sequence binds at one side of the promoter, polymerase will be able to extend single-stranded DNA but if it binds to another side of the promoter, extension is stopped (due to polymerase being unable to extend 3′→5′ direction). However, if there is sufficient primer, another primer molecule can bind to the correct place and DNA polymerase can fill the gap in between the bound primers. Bst DNA polymerase has high strand displacement activity so the wrongly annealed RNA oligonucleotide should be displaced. However, even if it was not displaced, T7 RNA polymerase should be able to generate transcripts from RNA-DNA hybrid.

It will be appreciated that if the amplification reaction is designed to amplify more copies of an original RNA signal sequence, the amplification probe could be designed such that RNA signal sequence that binds to the part of the amplification probe that follows the promoter sequence does not inhibit or interfere with the primer extension reaction.

In the research of Arnaud-Barbe et al. (Nucleic Acids Research. 26:3550-3554, 1998), they tested transcription by T7 polymerase with templates of a dsDNA promoter followed by an RNA region in which the transition from DNA to RNA occurs 18 bases downstream from the promoter sequence. This design was based on the observation that T7 RNA polymerase undergoes the transition from an unstable initiation complex to a stable elongation complex after the synthesis of 8-12 nt of nascent RNA. The presence of an RNA template in the elongation region did not appear to affect initiation, the conformational change or the elongation steps.

Thus, it has been found that if the RNA signal sequence to be generated from the amplification probe has 12-14, or more, additional nucleotides immediately following the promoter region, primer extension can proceed from the 3′ end of the RNA signal sequence to produce the double-stranded promoter, the 12-14 (or more) additional nucleotides and that this extra sequence is enough to overcome any hybridisation that has occurred between the RNA signal sequence and the one encoded by the amplification probe after the promoter sequence. In this way, it is possible to generate a first RNA signal sequence from the 3WJ and a second RNA signal sequence which comprises the same sequence as the first but has an additional 12-14 (or more bases) at the 5′ end. By careful design of the sequence on the riboregulator switch that the oligonucleotide trigger (e.g. RNA signal sequence) hybridises to, either or both the original RNA signal sequence and the one that has been extended can hybridise to the riboregulator switch and effect signal generation.

In one embodiment, the amplification probe is single-stranded.

In one embodiment, the amplification probe comprises DNA nucleotides.

In one embodiment, the amplification probe comprises or consists of the sequence disclosed in SEQ ID NO: 3, 39 or 40.

In one embodiment, the nucleic acid amplification probe is present at the same time as the sample is contacted with the first and second probes.

Riboregulator Switch [Section RS]

The invention can be performed using any riboregulator switch, including those comprising a toehold domain.

A toehold switch (or toehold riboregulator) is an example of a suitable riboregulator switch. The important feature of the riboregulator switch is that binding of the RNA trigger sequence to the riboregulator switch effects a conformational change on the riboregulator switch such that the previously constrained RBS is accessible to ribosomes allowing translation of the reporter gene product. The RNA trigger sequence can therefore bind a toehold domain, a partially or fully double-stranded stem domain or any other part of the riboregulator switch, such as the loop of the hairpin.

WO 2014/074648 (President and Fellows of Harvard College and Trustees of Boston University) describes programmable toehold riboregulators that can be activated by RNAs and can be used or adapted for use in the present invention.

In one embodiment, the riboregulator switch sequence comprises a fully or partially double-stranded stem domain, a loop domain and distinct single-stranded domains.

In one embodiment, the single-stranded domain capable of hybridising to the RNA trigger sequence is a toehold domain. In another embodiment, the single-stranded domain capable of hybridising to the RNA trigger sequence is part of the loop domain in the riboregulator switch. The toehold domain initiates the interaction of the hairpin with the trigger oligonucleotide via linear-linear binding.

The structure/conformation of the riboregulator switch is such that when in the inactive state the RBS cannot be utilised by the ribosome to effect translation of the reporter gene product. Upon activation, the hairpin loop structure is disrupted/rearranged such that the ribosome can bind to the RBS and effect translation of the reporter gene product. Activation generally occurs when an oligonucleotide hybridises to part of the single-stranded and part of the double-stranded region. With riboregulator switches that have a toehold domain the trigger oligonucleotide binds to the single-stranded toehold domain and part of the stem of the riboregulator sequence and this then leads to an alteration in the structure of the riboregulator switch which then permits ribosome binding and translation to produce the reporter gene product.

The riboregulator switch sequence is an RNA sequence that is part single-stranded and part double-stranded, by virtue of part of it annealing to itself to form a hairpin loop structure. With toehold switches, at the 5′ end there is a single-stranded toehold domain that can hybridise with some of the oligonucleotide trigger sequence. Typically, next there is a fully or partially double-stranded stem domain formed when two complementary stretches of the nucleic acid sequence hybridise together creating an intervening loop domain (stem and loop together forming a hairpin loop) then at the 3′ end there is a further single-stranded domain. The hairpin structure and subsequent single-stranded domain comprises a RBS sequence, an initiation codon (translational start site) and a coding domain for a reporter gene arranged such that the ribosome is only able to effect translation when the hairpin loop structure has been partially or fully disrupted following binding of part of the oligonucleotide trigger sequence to the toehold domain and part of the oligonucleotide trigger sequence binding to a fully or partially double-stranded stem domain. Thus, in the inactive form, because of the intact hairpin loop structure ribosomes are unable to bind to the sequestered RBS or if they can bind are unable to effect translation. The RBS can be located in all or part of the stem domain and/or all or part of the loop domain. Within the stem domain there can be one or more regions where the two sequences are not complementary forming small bulge regions. The initiation codon may be located in such a bulge region.

A representation of a toehold switch is shown in FIG. 5A. The example in FIG. 5A shows the location of the toehold sequence, a double-stranded stem, a bulge, further double-stranded stem and loop, with the RBS and initiation codon (translational start site) indicated; and at 3′ end (right hand side) a single-stranded protein coding sequence for lacZ. The probe adopts a hairpin loop structure in view of region of complementarity.

FIG. 5B illustrates the reactions that occur when part of the oligonucleotide trigger sequence (labelled as signal RNA 1) binds to the toehold domain sequence. Upon said binding the hairpin loop structure is disrupted which results in the RNA sequence for the RBS, initiation codon and lacZ protein coding sequence being single-stranded. When the RBS becomes accessible by ribosomes in the reaction mixture, translation of the lacZ protein product proceeds. In this set-up, lacZ protein is able to convert chlorophenol red-β-D-galactopyranoside substrate (yellow/orange in colour) to chlorophenol red (purple in colour) (see FIG. 5C). The colour change signifying that the target nucleic acid sequence of interest is in the test sample.

In one embodiment, some or all of the RBS is located in the double-stranded part of the stem domain of the toehold switch sequence.

In one embodiment, particularly with toehold switches, some or all of the RBS is located in the loop.

In one embodiment, the start/initiation codon is located in the stem or in the bulge of the stem.

In one embodiment, the oligonucleotide trigger sequence binds to some of the single-stranded toehold domain.

In one embodiment, the oligonucleotide trigger sequence binds part of the double-stranded stem domain of the riboregulator switch sequence.

In a particular embodiment, following binding of the oligonucleotide trigger sequence to the riboregulator switch sequence the hairpin structure of the riboregulator switch sequence is disrupted allowing the ribosome to bind to the RBS and effect translation of the reporter gene product.

Exemplary RBS sequences for use in the present invention include, but are not limited to, AGAGGAGA (or subsequences of this sequence, e.g., subsequences at least 6 nucleotides in length, such as AGGAGG). Shorter sequences are also acceptable, e.g., AGGA, AGGGAG, GAGGAG, etc. Numerous synthetic ribosome binding sites have been created, and their translation initiation activity has been tested. In various embodiments, any naturally occurring RBS may be used in the riboregulator switch molecule. In one embodiment, the RBS comprises the sequence: AGAGGAGA (SEQ ID NO: 19).

The most commonly utilized initiation codon sequence is AUG but alternative codons such as GUG or UUG could also be used.

In another embodiment, the initiation codon sequence is located in a non-complementary bulge region within the stem domain.

A reporter gene product is a protein (reporter protein) that can be detected either directly (e.g. through fluorescence) or indirectly (e.g. through its catalysis of a chemical reaction or triggering of detectable downstream genetic events) such that detection of the protein indicates production or activation of that protein.

In the context of the invention, reporter proteins are typically used to visualize activation of the riboregulator switch. Reporter proteins suitable for this purpose include but are not limited to fluorescent or chemiluminescent reporters (e.g., green fluorescent protein (GFP) variants, luciferase, e.g., luciferase derived from the firefly (Photinus pyralis) or the sea pansy (Renilla reniformis) and mutants thereof), enzymatic reporters (e.g., β-galactosidase, alkaline phosphatase, DHFR, CAT), etc. The eGFPs are a class of proteins that has various substitutions (e.g., Thr, Ala, Gly) of the serine at position 65 (Ser65). The blue fluorescent proteins (BFP) have a mutation at position 66 (Tyr to His mutation) which alters emission and excitation properties. This Y66H mutation in BFP causes the spectra to be blue-shifted compared to the wtGFP. Cyan fluorescent proteins (CFP) have a Y66W mutation with excitation and emission spectra wavelengths between those of BFP and eGFP. Sapphire is a mutant with the suppressed excitation peak at 495 nM but still retaining an excitation peak at 395 and the emission peak at 511 nM. Yellow FP (YFP) mutants have an aromatic amino acid (e.g. Phe, Tyr, etc.) at position 203 and have red-shifted emission and excitation spectra. In one embodiment, the protein coding sequence of the reporter gene is located in the further single-stranded domain of the riboregulator switch sequence.

In one embodiment, the reporter gene is fluorescent, luminescent or colourimetric.

In one embodiment, the reporter gene is a green fluorescent protein (GFP). Numerous green fluorescent protein variants are known.

In one embodiment, the reporter gene is LacZ (β-galactosidase) enzyme.

In another embodiment, the reporter gene is a partial LacZ enzyme (e.g. α-peptide), while another part of the enzyme (e.g. w-peptide) is provided separately.

In one embodiment, the reporter gene is luciferase.

In one embodiment, the production of LacZ enzyme is detected by contacting with the enzyme substrate chlorophenol red-β-galactopyranoside and detecting colour change.

In one embodiment, the reporter gene encodes T3 RNA polymerase. A T3 promoter, which is recognised by T3 RNA polymerase, is placed upstream of genes encoding each of a flavonoid 3′,5′-hydroxylase enzyme, a flavonoid 3′-monooxygenase enzyme, a naringenin 3-dioxygenase enzyme, a bifunctional dihydroflavonol 4-reductase/flavanone 4-reductase enzyme and a leucoanthocyanidin dioxygenase enzyme, such that production of the T3 RNA polymerase causes production of these enzymes. The T3 RNA polymerase would transcribe these genes from DNA that could be supplied as linear DNA or on plasmid(s). These enzymes then act together to convert naringenin (colourless) into cyanidin (violet). In an adaptation of this, the riboregulator switch could encode one of these enzymes, the other enzymes and the naringenin substrate could then be supplied to the reaction site. Only if the target sequence is present and the produced trigger oligonucleotide activates the riboregulator switch to release the enzyme reporter gene product would the substrate be converted to cyanidin.

There are a multitude of other options offered by biology. As examples, the reporter gene product could be a transcription factor that binds to a particular promoter sequence to encourage RNA polymerase to transcribe from that promoter; or it could be a transcription factor that binds to another protein that may have been preventing transcription, and binding changes the conformation of the second protein to allow transcription to occur.

In particular embodiments the riboregulator switch sequence molecule comprises a sequence selected from the group consisting of SEQ ID NO: 41, 42, 43 and 45, a sequence with at least 90% sequence identity thereto or a sequence with 1, 2, 3, 4, 5 or 6 substitutions therein.

According to another aspect of the invention there is provided a riboregulator switch molecule comprising a sequence selected from the group consisting of SEQ ID NO: 41, 42, 43 and 45, or a sequence with at least 90% sequence identity thereto.

According to another aspect of the invention there is provided a riboregulator switch molecule comprising a sequence selected from the group consisting of SEQ ID NO: 41, 42, 43 and 45, or a sequence with 1, 2, 3, 4, 5 or 6 substitutions therein. By a substitution therein we mean that one of the nucleobases has been substituted by another (e.g. adenine for cytosine).

In particular embodiments, the methods of the invention can utilise any of the sequences disclosed in Table 1.

It will be appreciated that some amount of non-specific reporter gene product production may arise. The method can therefore be carried out using positive and negative control samples to verify positive test results.

In another aspect of the invention there is provided a nucleic acid sequence which comprises a sequence complementary to an oligonucleotide trigger sequence, a RBS, an initiation codon and a reporter gene arranged in riboregulator switch structure.

The methods of the invention require de novo nucleic acid and/or protein synthesis, e.g. chain extension using DNA polymerase, transcription using RNA polymerase and translation using ribosomes. As such, DNA polymerase and RNA polymerase and the reagents and buffers needed to facilitate the chain extension, transcription and translation, such as ribo- or deoxyribo-nucleotide triphosphates and cell-free extract comprising ribosomes must be present. In addition, for detection purposes an enzyme substrate for the reporter gene product may also be needed. For example, LacZ cleaves the yellow substrate, chlorophenol red-β-D-galactopyranoside to produce a purple chlorophenol red product that is visible to the naked eye and can be measured on standard plate readers by monitoring the absorbance at 570 nm. Another example is chitinase which cleaves a colourless substrate (4-nitrophenyl N,N′-diacetyl-β-D-chitobioside) to yield a yellow p-nitrophenol product. The colourimetric output is visible to the naked eye and can be quantified using a standard plate reader at 410 nm (see, e.g. Pardee, et al., (2014, ibid).

Cell-free protein production can be accomplished with several kinds and species of cell-free extract, such as E. coli, insect, wheat-germ and mammalian. Cell-free extracts suitable for use in the present invention are available from various commercial sources.

Common components of a cell-free reaction include a cell extract, an energy source, a supply of t-RNAs and amino adds and cofactors such as magnesium. A cell extract can be obtained by lysing the cell of interest (e.g. bacterial cells) and removing the cell walls and other debris by centrifugation. What remains are the necessary cell machinery to effect cell-free protein synthesis (CFPS), including ribosomes, aminoacyl-tRNA synthetases, translation initiation and elongation factors, nucleases and cellular components needed for correct protein folding.

Ribosomes, tRNAs and other reagents needed to allow translation of the reporter gene product can be found in cell-free systems that are commercially available such as PURExpress® In Vitro Protein Synthesis Kit (NEB). PURExpress® is a reconstituted protein synthesis system based on the PUREsystem™ (Shimizu et al., Nat Biotechnol., 19:751-755, 2001) where all necessary components needed for in vitro transcription and translation are purified from E coli using His-tags.

The Complete System

In part, the invention requires formation of an oligonucleotide trigger sequence (such as via a 3WJ) followed by a riboregulator switch detection reaction, with an optional sequence amplification reaction to amplify up the oligonucleotide trigger sequence.

In one embodiment, formation of a 3WJ with production of an oligonucleotide trigger sequence, with or without the optional oligonucleotide trigger sequence amplification reaction, of the methods of the invention are carried out at the same time. Thus, the step of allowing the oligonucleotide trigger sequence molecules to come into contact with the riboregulator switch probe is facilitated by virtue of the riboregulator switch probe already being present in the reaction mixture that the test sample is applied to/contacted with.

In one embodiment, formation of the 3WJ with production of an oligonucleotide trigger sequence is carried out in a first reaction phase and then the reaction product from this first reaction phase is brought into contact with the riboregulator switch sequence and the signal generation reaction is carried out in a second reaction phase. In one embodiment, a two-step reaction is performed where the formation of the 3WJ is carried out in a first reaction phase, and signal amplification and reporting reaction with the riboregulator switch is carried out in a second reaction phase.

In another embodiment, a three-step reaction is performed where the formation of the 3WJ is carried out in a first reaction phase, signal amplification is carried out in a second reaction phase and the reporting reaction with the riboregulator switch is carried out in a third reaction phase.

In one embodiment, the reactions are carried out at a temperature of 55°-70° C. for a period of time.

In one embodiment, reactions are carried out at a temperature of 65° C.+/−2° C. for a period of time.

In one embodiment, the reactions are carried out at a temperature of 41° C.+/−2° C. for a period of time.

The length of time required for the reactions to complete and deliver a visible signal will depend on factors such as the amount of reagents, the amount of target nucleic acid present in the sample and the reaction temperature. The amounts of the particular reagents will need to be combined in quantities which maximise the overall reaction. In other words, the optimum reaction conditions for the DNA polymerase may not be the same as that needed by the RNA polymerase, or the ribosomes (in translation), or the reporter gene product reaction. It is likely that a compromise set of reaction conditions will be required that allows each of the essential reactions to proceed efficiently, albeit at less than maximum level. The person skilled in the art can devise such conditions using routine experimentation. It will be appreciated that the longer the reaction time allowed the greater the amount of reporter gene product that could be generated. In one embodiment, all the reactions from template detection to signal (reporter protein) detection are carried out within a period of time of 10-300 minutes. Conveniently around 3-4 hours. However, it will be appreciated that the reaction time can be altered to suit the conditions.

In a particular embodiment, the various probes and other reagents such as polymerases, required to permit amplification, RNA signal sequence production, and translation of the coding domain for the reporter gene are provided on a solid substrate.

In one embodiment, the solid substrate is a semi-porous substrate.

In a particular embodiment, the substrate is a paper-based product such as a card and the probes and reagents to facilitate the reactions have been applied to the card in a dried or lyophilised form.

In another embodiment, the substrate comprises plastic, polymer-based, hydrogel, glass, silicon, quartz or microfiber.

In another embodiment, the distinct reaction steps are carried out in a microfluidic device.

In another embodiment, some of the reaction components, such as the nucleic acid molecules are bound to a zone in a microfluidic device and the test sample and various reaction reagents are applied to the nucleic acid molecules to initiate a particular reaction (e.g. primer extension using DNA polymerase, transcription using RNA polymerase or translation using cell-free extract). After suitable reaction times the fluids can be washed off and new reaction reagents applied to initiate the next reaction. In this way, a series of reactions can be carried out sequentially.

In another arrangement, a nucleic acid containing test sample is used to rehydrate detection components that have been dried or lyophilised onto plastic/paper or other suitable support medium. The reaction mix is incubated for a suitable period of time and at a suitable temperature (e.g. 30 min at about 41° C.). Optionally, the reaction mix is then transferred (e.g. by pipette) to a different site which contains enzymes lyophilised onto plastic/paper or other suitable support medium to facilitate the signal amplification reaction. The reaction is incubated for a suitable period of time and at a suitable temperature (e.g. 2 h at about 41° C.) to allow production of the trigger signal RNA. The reaction mix can then be transferred to a different site containing dried or lyophilised reporting reagents. The reaction is incubated for a suitable period of time and at a suitable temperature (e.g. 1 h at about 41° C.) to allow a visible colour change to be observed if the test sample included the target nucleic acid. This system employs sequential and modular reactions: target detection, signal amplification and signal reporting. In particular embodiments of the aspects of the invention each of these modular reactions can be carried out at the same location on a substrate, or at different locations on a substrate, such as where the reaction product from the first reaction (e.g. target detection) is transferred to the second reaction (e.g. signal amplification) and the reaction product from this is transferred to the next reaction module (e.g. signal reporting).

Lyophilization, or freeze-drying, is a method for the preservation of labile materials in a dehydrated form. It is particularly suitable for high-value labile biomolecules such as proteins. The process involves the removal of bulk water from a frozen protein solution by sublimation under vacuum with gentle heating (primary drying). This is followed by controlled heating to more elevated temperatures for removal of the remaining “bound” water from the protein preparation (secondary drying). Other drying methods can also be employed.

In one embodiment, the solid support is a porous substrate, and the shelf-stable composition is partially or completely embedded in the porous substrate.

The solid support can be in any form including, but is not limited to, a well, a tube, a planar substrate (e.g., a chip or a plate), a sphere, a porous substrate (e.g., a mesh or a foam), a 3D scaffold, a patterned surface (e.g., nano-patterns, or micro-patterns, or both), a porous or solid bead, a hydrogel, a channel (e.g., a microfluidic channel), a smooth surface, and a rough surface. In a preferred embodiment, the solid support is hydrophilic and preferably porous.

Paper is an extremely cheap and promising material for microfluidic chips. it is slender, easy to stock, employ and transport. It is compatibie with biological samples and can be chemically treated to bond with molecules or proteins and is environmentally friendly.

Particular paper-based substrates include paper and card. The substrate, e.g. card, can be any size but is conveniently small enough to be portable and/or held in the hand. A5 size and below would be convenient.

In one embodiment, the porous substrate comprises paper.

In one embodiment, the porous substrate comprises quartz microfiber, mixed esters of cellulose, porous aluminium oxide, or a patterned surface.

In one embodiment, the solid support comprises 1 or more, such as 2, 3, 4, 5, 6, 7, 8, 9, 10, 15, 20 etc., spatially distinct reaction regions where the reaction reagents are confined.

In one embodiment, the solid support is pre-treated with bovine serum albumin, polyethylene glycol, Tween-20, Triton-X, milk powder, casein, fish gelatin, or a combination of one or more thereof. Without wishing to be bound by theory, this pre-treatment step can increase the signal-over-noise ratio for a fluorescent signal by limiting non-specific binding and/or irreversible binding of the reaction components.

According to another aspect of the invention there is provided a solid substrate comprising one or more zones with reagents attached thereon, said reagents comprising: a first probe and a second probe capable of creating a three-way junction with a target sequence of interest and a riboregulator switch sequence probe. In particular embodiment, the substrate also has one or more of the following attached thereon in the zone: a DNA polymerase, an RNA polymerase, ribo-nucleotide triphosphates, deoxyribo-nucleotide triphosphates, a cell-free extract comprising ribosomes and an enzyme substrate reagent. In one embodiment, all the reagents are present within one zone. In another embodiment, some of the reagents are at one zone and others are at one or more other zones.

In a particular embodiment, the solid substrate also comprises an amplification probe. In a particular embodiment, the solid substrate also comprises one or two facilitator probes. In a particular embodiment, the reagents are applied to the substrate in dried or lyophilised form such that when they are reconstituted by addition of a fluid the reagents can move freely in the fluid.

In one embodiment, the solid substrate is paper-based, such as card.

The reactions of the invention can be carried out in one zone/location on the substrate. The fluid test sample is applied to the zone containing the reagents and the reactions allowed to proceed. The fluid can also be applied to other zones that contain control reactions. After the incubation period the results can be read by visualisation of, for example a colour change.

In one embodiment, the probes and reagents are provided at distinct zones on the solid substrate so that the reactions of the method can be carried out in different locations. For example the reaction leading to formation of the 3WJ could be carried out at one site, the primer extension to produce the double-stranded RNA promoter or double-stranded restriction enzyme recognition sequence could be carried out at the same site as formation of the 3WJ or at another site, amplification of the single-stranded oligonucleotide to produce the oligonucleotide trigger sequence could be carried out at a different site and the riboregulator switch signal reaction at another site. Product/fluid from a one reaction can then be transferred to the site of the next reaction, such as via a pipette or by lateral flow etc.

The reaction components can also be contained in, and the reactions themselves carried out in a microfluidic device. This would allow the different phases of the reaction, such as 3WJ oligonucleotide trigger sequence synthesis, amplification and riboregulator switch detection to take place in distinct zones with the initial sample fluid applied in one zone and the sample liquid and reaction products arising from each reaction zone being capable of moving to another zone where the next reaction can take place. In this way, the different reaction components (such as polymerases, buffers, bases etc.) can be located in distinct zones to minimise interference in the reaction by components that do not participate in the reaction (e.g. chain elongation, transcription, translation). Thus, for example, a SMART reaction can take place in one zone that houses the components necessary to effect the SMART reaction, the reactant fluid can then pass or flow to a second zone where, for example, RNA signal amplification can take place. The reactant fluid could then pass to another zone containing the third probe (riboregulator switch probe) and reaction components to effect translation of the reporter gene product; and the reactant fluid could then pass to another zone where the substrate for the reporter product is located and the result visualised. Of course, it will be appreciated that distinct reaction steps can be separated in this way or combined in one or other zones, such that, for example, the initial 3WJ reaction and RNA signal amplification could be carried out in zone 1 and the detection (using riboregulator switch probe and reporter gene product reaction) carried out in another zone; the two zones being connected by a channel through which fluid from the first zone can pass to the second zone.

Microfluidic approaches, devices and systems have been around since the 1950s. Various techniques can be employed to fabricate devices for microfluidics. For example, it is possible to use photolithography, soft lithography, thermoforming or etching techniques.

According to a further aspect of the invention there is provided a solid substrate comprising one or more zones of lyophilised or dried reagents, said reagents comprising one or more of the following: a first probe and a second probe capable of creating a three way junction with a target sequence of interest, a riboregulator switch sequence, an RNA amplification probe; one or two facilitator probes; a DNA polymerase, an RNA polymerase; ribo- or deoxyribo-nucleotide triphosphates; an enzyme substrate reagent; buffers; ribosomes; and cell-free extract comprising translational machinery such as ribosomes and other factors.

In one embodiment, the solid support comprises one or more fluidic channels (e.g., microfluidic channels) that connect reaction regions with an area for adding an aqueous sample (e.g. test sample). In this embodiment, when an aqueous sample is added to the area, the fluid is wicked away to the reaction regions, thereby a plurality of reaction regions can be activated by the same sample.

The sample can be any sample where nucleic acid can be found, such as body tissues and fluids, including: blood, plasma, serum, bile, amniotic, cerebrospinal fluid, lymph, pleural, pus, semen, sputum, saliva, bronchoalveolar washings, sweat, tears, vomit, urine, milk and faeces. In order to release their nucleic acid, cells may need to be lysed (e.g. in the presence of lysis buffer).

A useful feature of the present system is that the target sequence can be any nucleic acid (RNA or DNA) sequence of interest, such as a sequence from a pathogen (like Mycobacterium bovis or bovine viral diarrhoea virus), or a sequence of a particular mammalian or plant allele, such as the genotype of an individual could be determined. The system can be used to distinguish between allelic variations (such as gene mutations), which may be useful in the diagnosis of diseases. Through the use of small probes the process of the present invention can be used to detect single nucleotide differences (such as single nucleotide polymorphisms or single point mutations) in genetic material using stringent hybridisation conditions to ensure the binding of the first or second probe to the target sequence is achieved over binding to a target sequence where there is mismatch (perhaps as present in a normal genetic sequence). Thus, depending on the use it is to be put to the sequence of interest can be very long, such as detecting the presence of a pathogen in a sample, or small, such as for detecting single point mutations.

A particular unique sequence especially useful in the present invention is provided by bases 791-820 of 16S ribosomal RNA from Streptomyces brasiliensis (Stackebrandt et al., Appl. Environ. Microbiol. 57:1468-1477, 1991), which sequence has no alignment with any known human DNA or DNA of a known human pathogen.

One of the benefits of the method of the present invention is that the riboregulator switch (probe 3) does not need to be configured each time. It is triggered by the same oligonucleotide trigger sequence. This reduces the additional time and expense needed to adapt a test system to different pathogens (target sequences) and the amount of intellectual effort needed in designing a suitable detection probes to match the oligonucleotide trigger sequence produced.

According to another aspect of the invention there is provided a kit for use in detecting the presence in a sample of a nucleic acid sequence of interest, the kit comprising probes 1, 2 (first and second probes) and a riboregulator switch sequence in accordance with claim 1, and appropriate packaging means.

According to another aspect of the invention there is provided a kit for use in detecting the presence in a sample of a nucleic acid sequence of interest, the kit comprising probes 1, 2 and a riboregulator switch sequence in accordance with claim 1, and facilitator probes 1 and 2 in accordance with claim 4, and appropriate packaging means.

In a particular embodiment, the kit further comprises instructions for use in performing the methods of the invention, e.g. as in claim 1.

In a particular embodiment, the kit further comprises one or more of the following: a DNA polymerase; an RNA polymerase; ribo-nucleotide triphosphates, deoxyribo-nucleotide triphosphates; cell-free extracellular extract, a restriction enzyme, detection reagents; buffers.

In a particular embodiment, the kit comprises the Bacillus stearothermophilus (Bst) DNA polymerase.

In a particular embodiment, the kit comprises the restriction enzyme Nb.Bsml.

In a particular embodiment, the kit comprises T7 RNA polymerase.

In a particular embodiment, the kit comprises a cell-free system comprising ribosomes. A cell-free system is a system that contains all the component necessary for in vitro protein production. It may include cell-free extracts or purified cellular components.

According to another aspect of the invention there is provided a trio of nucleic acid probes, the first probe comprising a portion substantially complementary to the sequence of interest and so capable of hybridising thereto, and a portion non-complementary to the sequence of interest but comprising the full-length sequence of a first strand of a double-stranded RNA promoter and a template signal sequence, the second probe comprising a portion substantially complementary to the sequence of interest and so capable of hybridising thereto, and a portion non-complementary to the sequence of interest but complementary to a part of that portion of the first probe which is non-complementary to the sequence of interest, such that the first and second probes are capable of hybridising to the sequence of interest in an adjacent or substantially adjacent manner, so as to allow complementary portions of the first and second probes to hybridise to each other, and the third probe being a riboregulator switch sequence possessing a hairpin structure comprising single-stranded and double-stranded domains comprising a single-stranded domain capable of hybridising with some or all of an oligonucleotide trigger sequence, a RBS an initiation codon and a coding domain for a reporter gene arranged such that a ribosome is only able to effect translation when the hairpin loop structure has been disrupted following binding of the oligonucleotide trigger sequence to the riboregulator switch sequence. In a particular embodiment, binding of the oligonucleotide trigger sequence to the riboregulator switch sequence causes disruption of the hairpin structure allowing the ribosome to access the RBS, triggering translation of the coding domain of the reporter gene and production of the reporter gene product. In one embodiment, the trio of probes are for use in a method of detecting a nucleic acid sequence of interest.

Other Aspects:

The inventors have devised toehold-containing riboregulator switch molecules with a new design, wherein the RBS is in the stem domain and the toehold domain is upstream of the stem domain. Such molecules can be used to detect the presence of any RNA signal sequence but are particularly useful in the methods of the present invention.

According to another aspect of the invention there is provided a riboregulator switch molecule which comprises a toehold domain, a RBS, an initiation codon and a reporter gene, wherein the molecule is formed from a single-stranded molecule that is capable of self-hybridising to form regions of single and double strands including a single-stranded toehold domain, a fully or partially double-stranded stem domain, and a single-stranded hairpin loop domain, wherein the RBS is located in the stem region and wherein binding of an oligonucleotide signal sequence partially to the toehold domain and partially to the stem domain effects a conformational change in the self-annealed riboregulator switch sequence molecule which allows production of the reporter gene product. In one embodiment the toehold domain is upstream of the RBS. In one embodiment the toehold domain is at the 5′ end of the molecule and is single-stranded. The individual domains and arrangement of these domains in the riboregulator switch molecule can be as described elsewhere herein, such as in [section RS].

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, embodiments described in conjunction with a particular aspect, embodiment or example of the invention are to be understood to be applicable to any other aspect, embodiment or example described herein unless incompatible therewith. The patent, scientific and technical literature referred to herein establish knowledge that was available to those skilled in the art at the time of filing. The entire disclosures of the issued patents, published and pending patent applications, and other publications, including sequence accession numbers, that are cited herein are hereby incorporated by reference to the same extent as if each was specifically and individually indicated to be incorporated by reference. In the case of any inconsistencies, the present disclosure will prevail. Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1 94); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, suitable methods and materials are described herein. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

### DESCRIPTION OF THE FIGURES

FIG. 1. A simplified overview of the Moduleic Sensing™ Biosensor

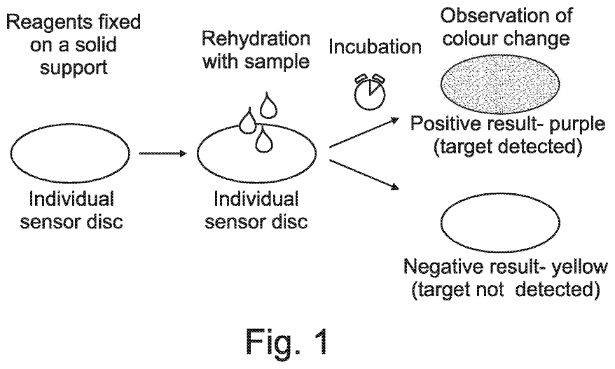

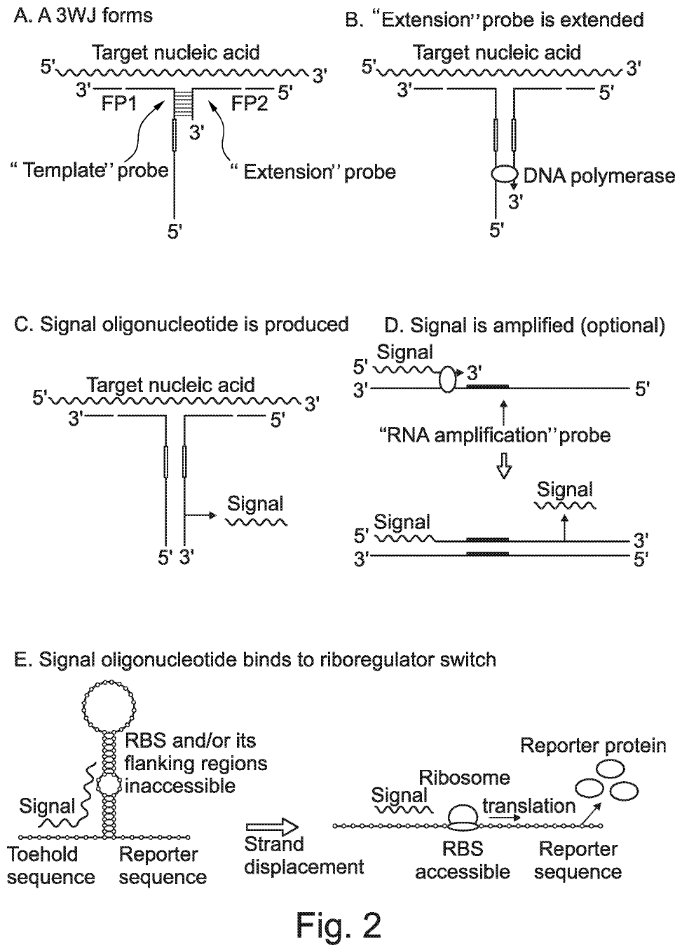

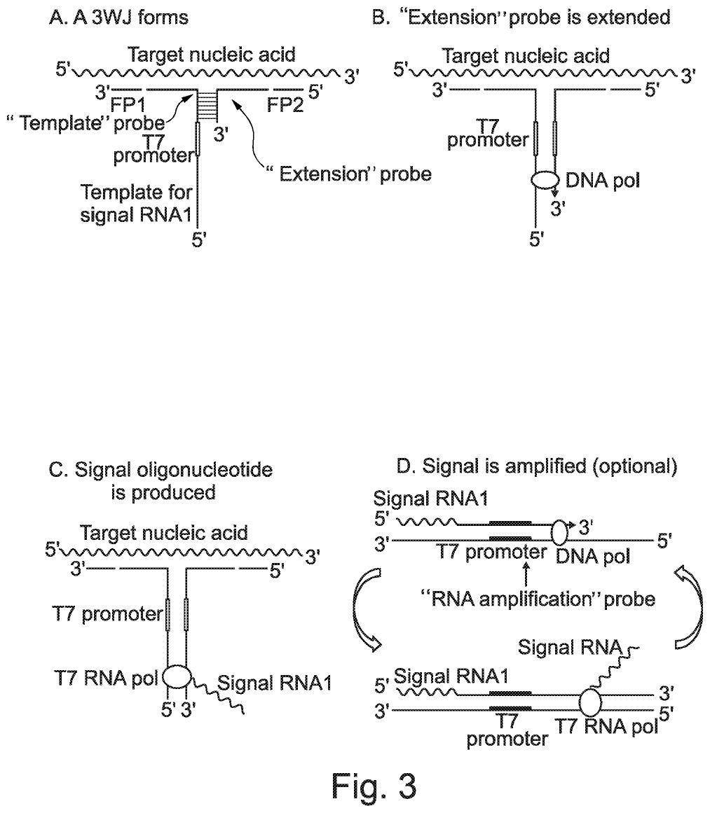

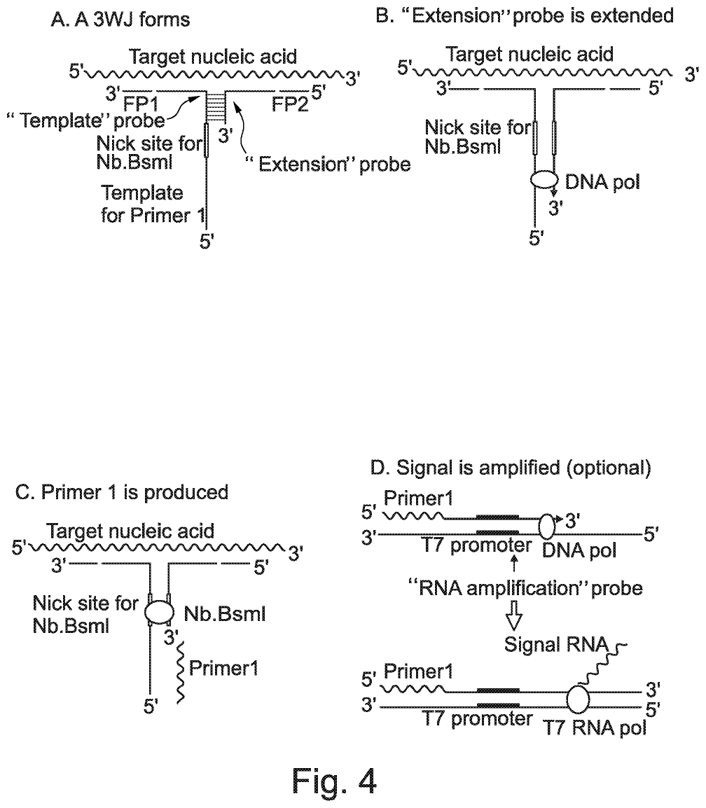

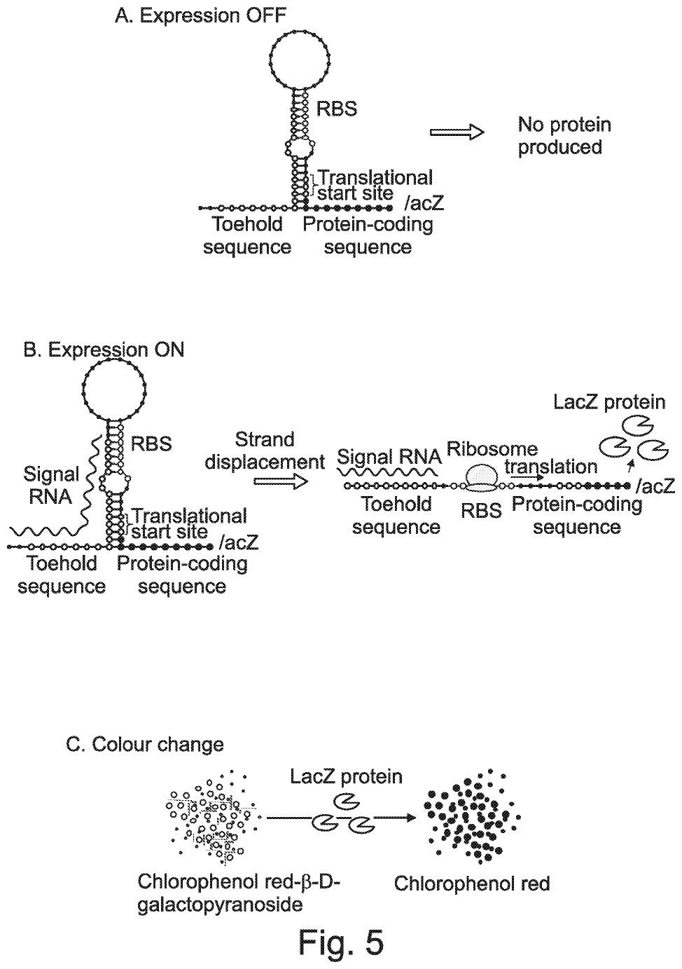

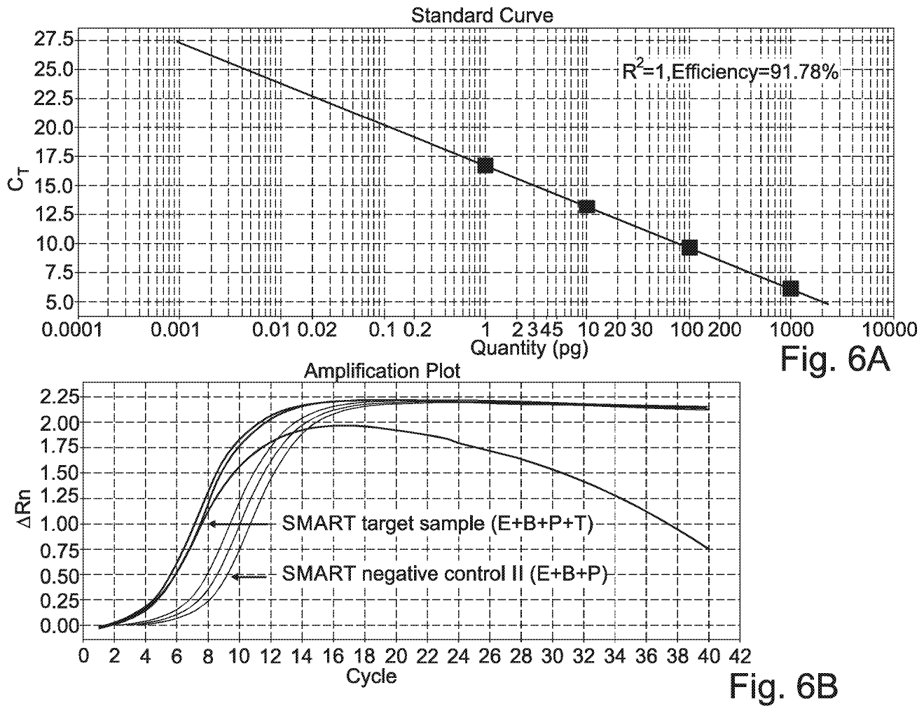

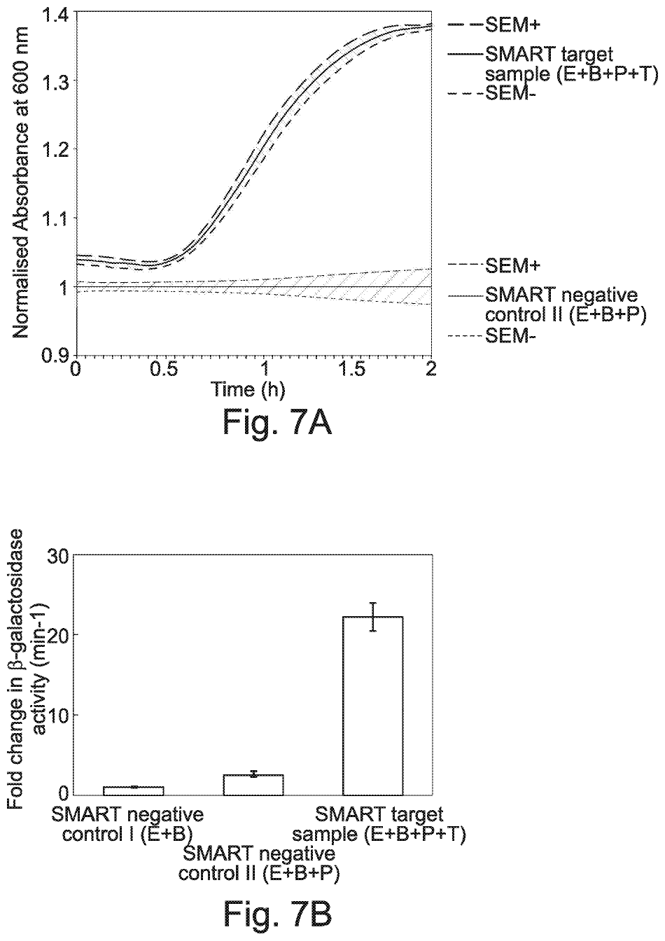

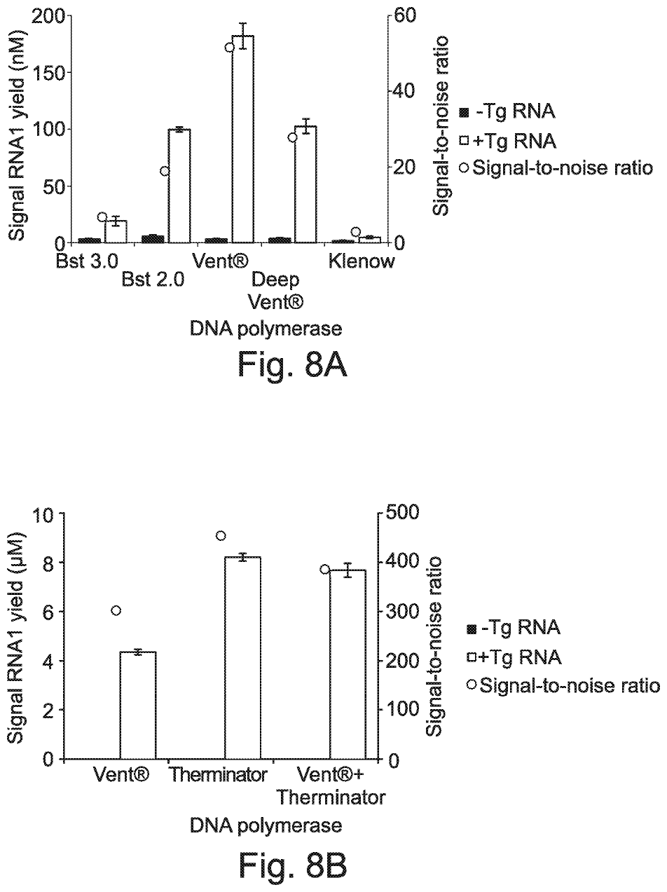

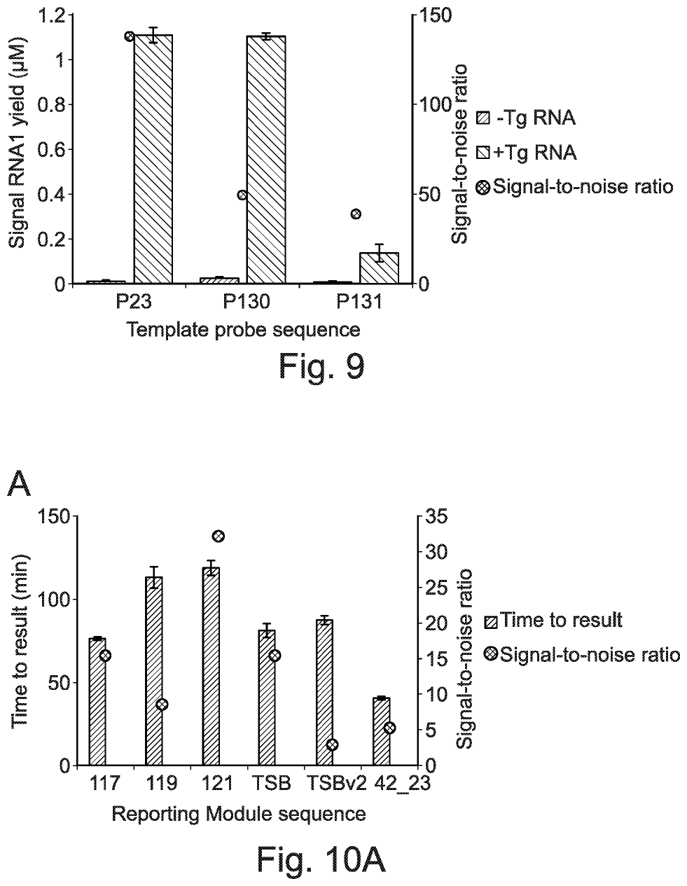

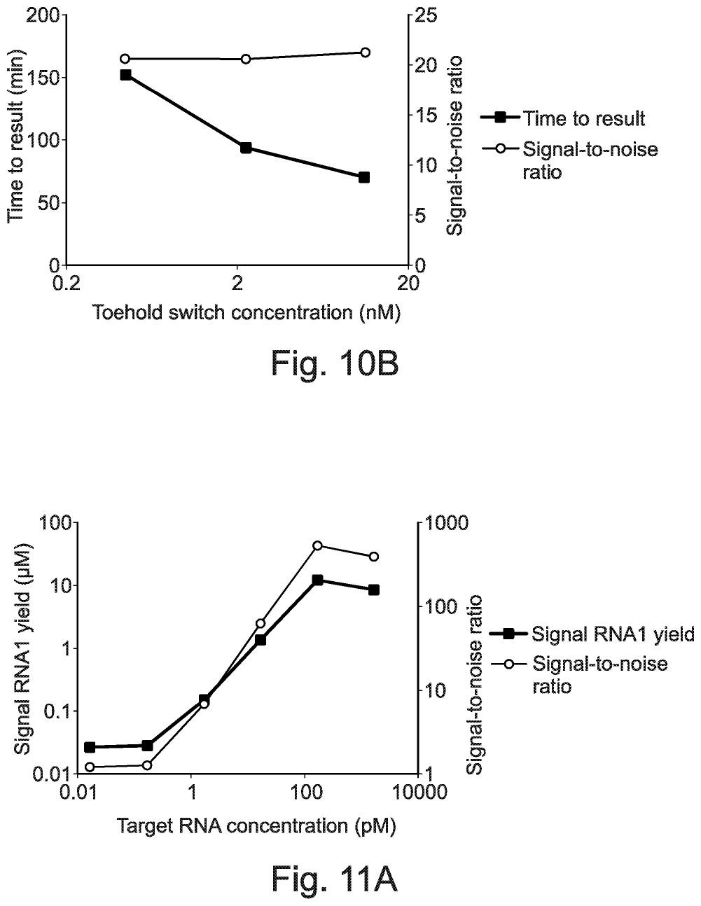

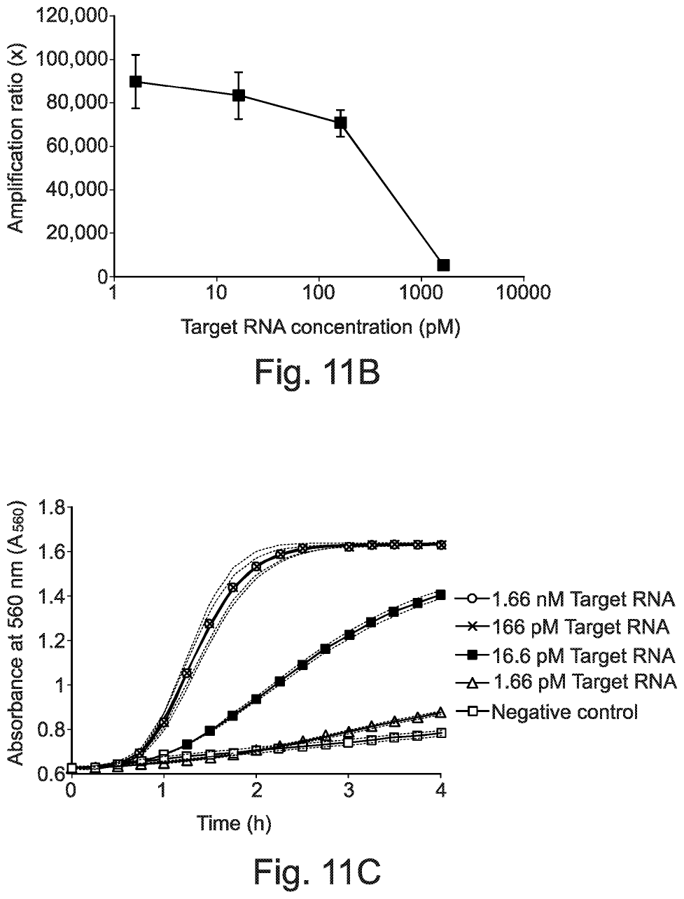

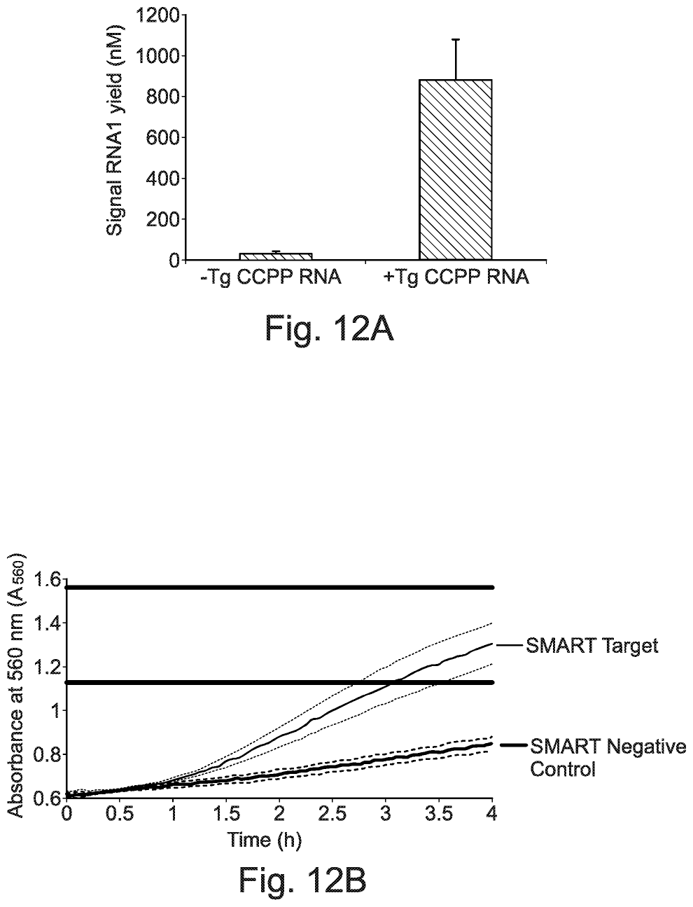

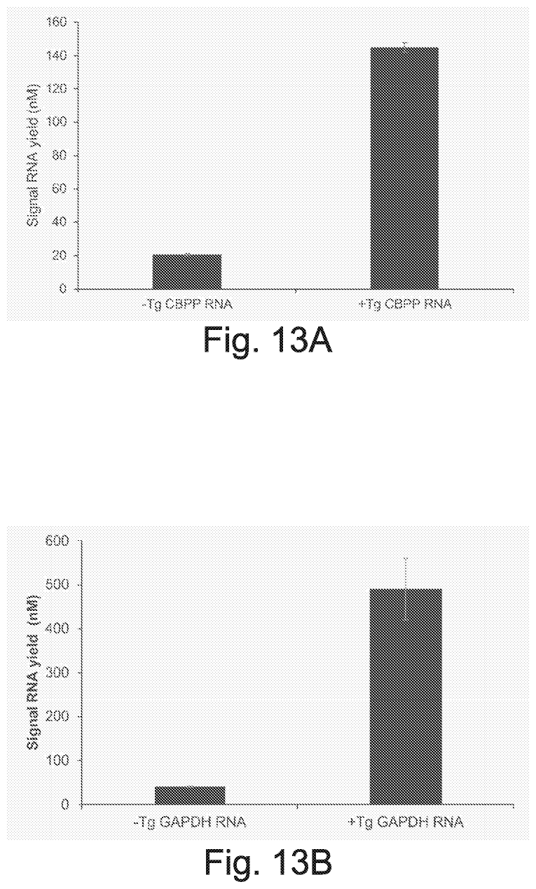

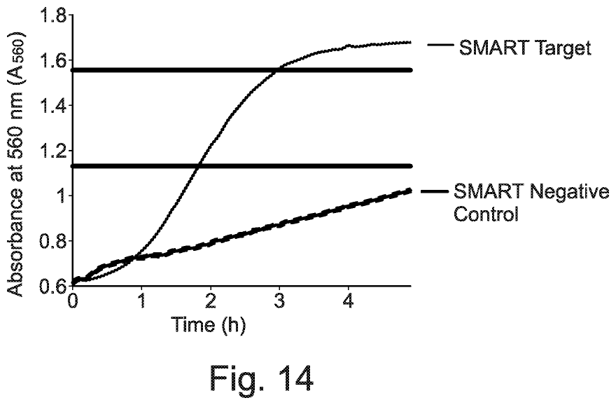

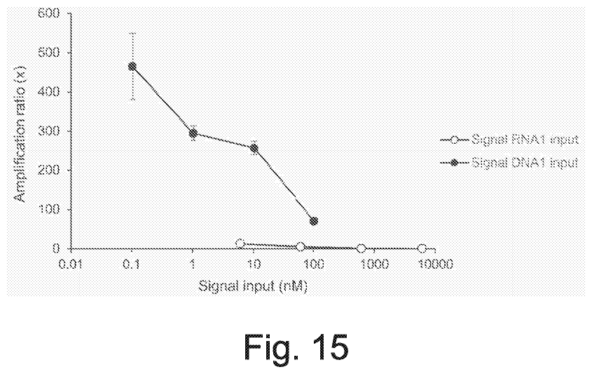

The components for isothermal nucleic acid amplification and detection are fixed onto a solid support. The components are activated by rehydration and the addition of a sample containing the target nucleic acid molecule. A colourimetric output is measured after an incubation period.

FIG. 2. Overview of three-way junction (3WJ) signal-mediated amplification and signal detection
A. The 3WJ structure comprises two single-stranded DNA probes (“template” and “extension”) that hybridise to both the target sequence and to each other. Optionally, two short facilitator probes (FP1 and FP2) are included to stabilise the 3WJ. The facilitator probes anneal to the target DNA or RNA sequence adjacent to the annealing sites of the “template” and “extension” probes.
B. Once a 3WJ is formed, DNA polymerase extends the complementary “extension” probe.
C. A single-stranded oligonucleotide (“signal”) is formed and released.
D. Optionally, the signal molecule anneals to an RNA amplification probe resulting in a chain reaction producing the same or a different “signal” molecule.
E. The signal molecule produced in step (C) or (D) binds to a riboregulator switch, affecting a conformational change within the riboregulator switch that allows the ribosome to bind the ribosome binding site (RBS) and affect translation of the reporter protein which can then be detected.

FIG. 3. Overview of signal-mediated amplification of RNA technology (SMART).

SMART is based on the formation of a “T” structure, known as a three-way junction (3WJ), which enables target-specific amplification of multiple signal RNA copies:
A. The 3WJ structure comprises two single-stranded DNA probes (“template” and “extension”) that hybridise to both the target sequence and to each other. Optionally, two short facilitator probes (FP1 and FP2) can be included to stabilise the 3WJ. The facilitator probes anneal to the target DNA or RNA sequence adjacent to the annealing sites of the “template” and “extension” probes. The “template” probe illustrated contains a single-stranded T7 promoter sequence; however, any RNA polymerase promoter can be used.
B. Once a 3WJ is formed, DNA polymerase extends the complementary “extension” probe, generating a functional double-stranded RNA polymerase promoter (e.g. T7).
C. This enables the binding of a T7 RNA polymerase to the double-stranded promoter and the subsequent transcription of an RNA signal (RNA1).
D. Optionally, the RNA1 signal molecule anneals to an RNA amplification probe resulting in a chain reaction of RNA1 signal amplification.

FIG. 4. Overview of three-way junction (3WJ) combined with nicking enzyme
A. The 3WJ structure comprises two single-stranded DNA probes (“template” and “extension”) that hybridise to both the target sequence and to each other. Optionally, two short facilitator probes (FP1 and FP2) are included to stabilise the 3WJ. The facilitator probes anneal to the target DNA or RNA sequence adjacent to the annealing sites of the “template” and “extension” probes. The “template” probe contains a single-stranded T7 promoter sequence.
B. Once a 3WJ is formed, a DNA polymerase extends the complementary “extension” probe, generating a double-stranded restriction enzyme (e.g. Nb.Bsml) nicking recognition site.
C. This enables the nicking restriction enzyme to nick the sense strand of the double-stranded DNA. The 3′ part of that nicked strand is then released as Primer 1. D. The Primer 1 signal molecule anneals to an RNA amplification probe resulting in a chain reaction producing Signal RNA.

FIG. 5. Detection of RNA using a riboregulator switch
A. In an inactive state, the ribosome binding site (RBS) of a lacZ gene transcript is sequestered in a hairpin structure, preventing its translation.
B. Translation requires the RNA trigger (Signal RNA1) to bind to the toehold domain sequence, initiating strand displacement and releasing the RBS from the hairpin. The ribosome is then able to bind to the newly accessible RBS and synthesise the LacZ (β-galactosidase) enzyme.
C. The LacZ enzyme cleaves a yellow-orange substrate, chlorophenol red-β-D-galactopyranoside, into a purple product, chlorophenol red.

FIG. 6. Quantification of RNA signal by RT-qPCR, following DNAse I treatment of SMART reactions and 1:100 dilution.
A. Quantities of signal RNA were estimated by RT-qPCR using primers SEQ ID NO:7 and SEQ ID NO:8 based on a 4-log standard curve from 1,000 pg to 1 pg. This is plotted against the threshold cycle (C_{T}) value generated by the RT-qPCR.
B. Graph shows amplification plot of SMART target sample (E+B+P+T), containing 25 fmol of target RNA and SMART negative control II (E+B+P) sample with no target. The quantities of signal RNA estimated to be in the target sample and in the negative control were 2,062±206 pg and 368±152 pg, respectively. ΔRn represents the change in fluorescence value measured during RT-qPCR.
   E—enzymes, B—buffers, P—probes, T—target RNA.

FIG. 7. Toehold switches and in vitro protein synthesis components were fixed onto filter paper, rehydrated with samples after SMART reaction and incubated at 41° C. Samples contained SMART negative control I (E+B), SMART negative control II (E+B+P) or SMART target sample (E+B+P+T) and toehold switch B.
A. Graph shows absorbance of SMART target sample (E+B+P+T) at 600 nm±standard error of the mean (n=3), normalised to the A₆₀₀ of SMART negative control II samples (E+B+P) without the RNA target. The increase in A₆₀₀ is a measure of the cleavage of the yellow-orange chlorophenol red-β-D-galactopyranoside into the purple chlorophenol red by β-galactosidase.
B. Graph shows the maximum rate of change in A₆₀₀ following addition of SMART negative control I (E+B), SMART negative control II (E+B+P) or SMART target sample (E+B+P+T) to toehold switch B during incubation at 41° C. This maximum rate of change occurred at approximately 54 min. The figure indicates that β-galactosidase enzyme was produced 20±2 times faster from the toehold switch with SMART target sample compared to SMART negative control with only probes (p<0.001).
   E—enzymes, B—buffers, P—probes, T—target RNA.

FIG. 8. The impact of different DNA polymerases on Signal RNA1 production in SMART.
A. Graph presents results from initial screening of five different DNA polymerases in the SMART assay. The SMART assay was performed using a bovine viral diarrhoea virus (BVDV) target sequence (SEQ ID NO:21) with probes SEQ ID NO:25 and SEQ ID NO: 26; the resulting signal RNA1 (SEQ ID NO:35) produced by SMART was treated with Exonuclease V DNase and quantified by RT-qPCR using primers SEQ ID NO:46 and SEQ ID NO:47.
B. Comparison of the performance of DNA polymerases individually and combined in a SMART assay using a Mycobacterium capricolum subsp. capripneumoniae (contagious caprine pleuropneumonia; CCPP) target sequence (SEQ ID NO:22). SMART reactions were then treated with Exonuclease V DNase and the resulting signal RNA1 (SEQ ID NO:35) quantified by RT-qPCR using primers SEQ ID NO:46 and SEQ ID NO:47. Signal-to-noise ratio is also indicated. Error bars indicate standard error of the mean (n=3).

FIG. 9. The impact of different Signal RNA1 sequences on their production by SMART.

The SMART reaction has been performed using BVDV target (Tg) RNA (SEQ ID NO:21), with extension probe SEQ ID NO:25 and BVDV-specific target probes SEQ ID NO:26, SEQ ID NO:27 or SEQ ID NO:28. The SMART reaction was set-up as described, using 0.22 U/μl Vent® exo-DNA Polymerase. SMART reactions were then treated with Exonuclease V and subjected to RT-qPCR using primer pairs SEQ ID NO:46 and SEQ ID NO:47, SEQ ID NO7 and SEQ ID NO:8, or SEQ ID NO:48 and SEQ ID NO:49 respectively for the three target probes. Signal RNA production is indicated in the absence (black) and presence (white) of target RNA in the presence of different template probes encoding different Signal RNA1 sequences. The signal-to-noise ratio is also indicated. Error bars indicate standard error of the mean (n=3).

FIG. 10. The impact of the toehold switch sequence on the time to generation of a purple colour and the signal-to-noise ratio.
A. DNA cassettes of toehold switches 117 (SEQ ID NO:41), 119 (SEQ ID NO:42), 121 (SEQ ID NO:43), B (SEQ ID NO:5), B version 2 (SEQ ID NO:44) and 42_23 (SEQ ID NO:45) comprising a transcriptional promoter, the toehold switch sequence and the lacZ coding sequence (SEQ ID NO:6) were incubated with cell-free transcription/translation reagents and their respective activating Signal RNA in liquid conditions. During the reaction, RNA polymerase transcribed the toehold switch and lacZ sequences. Binding of the Signal RNA to the toehold switch is intended to alter the RNA conformation, allowing the ribosome to translate the lacZ coding sequence and, subsequently, the colour-change reaction to occur. Graph indicates time to result and signal-to-noise ratios of the different toehold switches. Error bars indicate standard error of the mean (n=4).
B. The DNA cassette encoding a toehold switch 121 (SEQ ID NO:43) was added to the cell-free reaction at different concentrations in liquid conditions and the development of the purple colour was monitored over time at 560 nm. Error bars are included for the time to result (n=4), but the highly regulated nature of the particular toehold switch mean that these are not visible in the figure. Signal-to-noise ratio is also indicated.

FIG. 11. Dose-response curve for signal production.

The ability of the refined Moduleic Sensing™ assay to detect a 10-fold dilution series of CCPP target RNA (SEQ ID NO:23) using probes (SEQ ID NO:31) and (SEQ ID NO:32) was evaluated. SMART reaction was followed by Exonuclease V digestion and RT-qPCR using primers SEQ ID NO:46 and SEQ ID NO:47 to quantify the amount of Signal RNA1 (SEQ ID NO:35) produced. The cell-free assay in liquid conditions using toehold switch 121 (SEQ ID NO:43) was used to quantify the colour change resulting from the signal RNA1 production.
A. Signal RNA1 yields and signal-to-noise ratios resulting from the addition of Target RNA at different concentrations.
B. Amplification ratios at target RNA concentrations greater than the limit of detection. The amplification ratio is the ratio of Signal RNA1 produced to RNA target added. At target concentrations less than 1.7 pM the amplification ratio increases exponentially due to assay noise (data not shown), while at target concentrations higher than 166 pM the assay becomes saturated leading to a relative reduction in the amplification ratio. (Error bars indicate standard deviation, n=3.)
C. SMART samples from A (from 1.7 pM to 1.66 nM) were incubated with the cell-free reaction components in liquid conditions, using toehold switch 121 (SEQ ID NO:43). Negative control samples contained the SMART reactions without any target RNA. The results show samples with 166 pM and 1.66 nM of target RNA becoming red at approximately 80 min and purple at approximately 125 min. The 16.6 pM target RNA samples became red at approximately 160 min but did not become purple during the experiment. The 1.66 pM target RNA samples could be differentiated from the negative controls based on their A₅₆₀ values but not with the naked eye. These and the negative control samples remained yellow during the experiment. Full lines indicate mean values, while dashed lines indicate standard error of the mean (n=3). The upper horizontal line (A₅₆₀=1.56) represents a purple colour change, the lower horizontal line (A₅₆₀=1.13) represents an indistinct colour change.

FIG. 12. Diagnostic sensitivity and specificity in Moduleic Sensing™

SMART reactions were performed using CCPP target RNA (SEQ ID NO:23) with probes SEQ ID NO:31 and SEQ ID NO:32 on samples containing total RNA extracted from 250 μl of bovine plasma from a healthy animal using the QIAamp® viral RNA mini kit (52904, Qiagen) and either 17 pM of Target (Tg) RNA (SEQ ID NO:23) or no target RNA. The resulting signal RNA was either treated with Exonuclease V and quantified by RT-qPCR using primers SEQ ID NO:46 and SEQ ID NO: 47 (A) or added to the liquid cell-free assay with toehold switch 121 (SEQ ID NO:43) (B). The upper horizontal line (A₅₆₀=1.56) represents a purple colour change, the lower horizontal line (A₅₆₀=1.13) represents an indistinct colour change. Error bars and dashed lines indicate standard deviation (n=12).

FIG. 13: Production of signal RNA using probes designed against CBPP and GAPDH RNA.
A. SMART reactions were performed on samples in the absence or presence of 15.3 nM in vitro transcribed CBPP RNA sequence (SEQ ID NO:22). These SMART reactions were set up with CBPP-specific probes (SEQ ID NO:29) and (SEQ ID NO:30) and Therminator as DNA polymerase. Reactions were then treated with Exonuclease V and the resulting signal RNA (SEQ ID NO:35) was quantified by RT-qPCR using primers SEQ ID NO:46 and SEQ ID NO:47. Error bars indicate standard deviation (n=3 for each condition).
B. SMART reactions were performed on samples in the absence or presence of 15.3 nM in vitro transcribed GAPDH RNA sequence (SEQ ID NO:24). These SMART reactions were performed using 0.22 U/pl Vent ® DNA polymerase and GAPDH-specific probes (SEQ ID NO:33) and (SEQ ID NO:34) along with amplification probe SEQ ID NO:40. Reactions were then subjected to Exonuclease V treatment. The resulting signal RNA (SEQ ID NO:37) was quantified by RT-qPCR using primers SEQ ID NO:48 and SEQ ID NO:49. Error bars indicate standard deviation (n=3 for each condition).

FIG. 14. Signal generation from a two-step Moduleic Sensing™ assay.

A one-step SMART reaction was performed, by omitting the annealing step and adding probes (SEQ ID NO:31) and (SEQ ID NO:32) plus and minus target RNA (SEQ ID NO:23) directly with the components for the amplification reaction, as well as the toehold switch 121 (SEQ ID NO:43) DNA cassette. 1.5 μl of this reaction was added to the cell-free reaction, containing Solution A, Solution B and chlorophenol red-β-D-galactopyranoside substrate only. The resulting increase in absorbance at 560 nm is indicated when a SMART reaction in the presence of target RNA (SMART target) and the absence of target RNA (SMART negative control) was added. The upper horizontal line (A₅₆₀=1.56) represents a purple colour change, the lower horizontal line (A₅₆₀=1.13) represents an indistinct colour change.

FIG. 15. Fold-change observed when a synthetic signal DNA molecule is used in combination with the amplification probe.

Graph shows results of the incubation of the amplification probe SEQ ID NO:39 with in vitro transcribed signal RNA1 (SEQ ID NO:35) or signal DNA1 (SEQ ID NO:38). Signal RNA2 (SEQ ID NO:36), defined as a sequence produced from an amplification probe and different from signal RNA1 production is measured by RT-qPCR with primers SEQ ID NO:7 and SEQ ID NO:8. Results are defined as the ratio of the Signal RNA2 yield relative to the amount of input Signal RNA1 (open circles) or signal DNA 1 (filled circles) added. The addition of signal RNA1 resulted in no significant production of the Signal RNA2. However, when a DNA oligonucleotide primer (Signal DNA1) with the same sequence design as Signal RNA1 was used, substantial amplification of the signal was observed, with an amplification ratio of up to 465±85 x.

The invention will now be further described with reference to the following non-limiting examples, and the figures described above.

### EXAMPLES

1.1 Preparation of Oligonucleotides

DNA oligonucleotides, RNA oligonucleotides and double-stranded DNA fragments were synthesised commercially by Integrated DNA Technologies, Inc (IDT) or Eurogentec Ltd and resuspended in nuclease-free water at a concentration of 100 μM according to manufacturers' instructions.

1.1.1 List of Oligonucleotides and Other Sequences (Table 1)

RNA targets

| | |
|---|---|
| Target RNA from Hall et | GGGUUCUACAUUGAUGUUGGCAAUCUUCCAAAGGUAAA |
| al., 2002, ibid | AGCAGAACAAUACCUCAGAGAGGUAAUGGGACGUUACC |
| ssRNA | GCAACAAACUUGUUUAUGAUGCAAACACAGGUGAAAUCA |
| | AGGACGACAAGAAACAUAUGUCGAUGCUUGCUAGUUAU |
| | UGCUCAGCGG (SEQ ID NO: 1) |
| BVDV Target RNA | AUGGAGUUGAUCACAAAUGAACUUUUAUACAAAACAUAC |
| ssRNA | AAACAAAAACCCGCUGGAGUG (SEQ ID NO: 21) |
| CBPP Target RNA | UGAUGAAAAAAUUGACAAGCCAAGUCAUUCAGAUAAACC |
| ssRNA | ACAAGCAGAUGAUUCUAACAACAAUAGAGACAUUUU |
| | (SEQ ID NO: 22) |
| CCPP Target RNA | CACAAUUCGGAGUUUCACUAGAUAAAGUUGAUGCUACA |
| ssRNA | UUUUUAACAUCUCCUCAGC (SEQ ID NO: 23) |
| GAPDH Target RNA | GGGAUCCUGCCAACAUCAAGUGGGGUGAUGCUGGUGC |
| ssRNA | UGAGUAUGUGGUGGAGUCCACUGGGGUCUUCACUACCA |
| | UGGAGAAGGCUGGGGCUCACUUGAAGGGUGGCGCCAA |
| | GAGGGUCAUCAUCUCUGCACCUUCUGCCGAUGCCCCCA |
| | UGUUUGUGAUGGGCGUGAACCACGAGAAGUAUAACAAC |
| | ACCCUCAAGAUUGUCAGCAAUGCCUCCUGCACCACCAA |
| | CUGCUUGGCCCCCCUGGCCAAGGUCAUCCAUGACCACU |
| | UUGGCAUCGUGGAGGGACUUAUGACCACUGUCCACGCC |
| | AUCACUGCUAGUUAUUGCUCAGCGG (SEQ ID NO: 24) |

SMART detection and amplification

| | |
|---|---|
| Cyanophage N1 | TCGTCTTCCGGTCTCTCCTCTCAAGCCTCAGCGCTCTCTC |
| Template probe* | TCCCTATAGTGAGTCGTATTAATTTCGAAhACGTCCCATT |
| ssDNA | ACCTCTCTGAGGTATTGh (SEQ ID NO: 2) |
| Cyanophage N1 | GTTTGCATCATAAACAAGTTTGTTGCGGTATTCGAAAT |
| Extension probe | (SEQ ID NO: 20) |
| ssDNA | |
| BVDV Extension probe | CACTCCAGCGGGTTTTTGTTTGTATGTTCTAGATTATG |
| ssDNA (artificial) | (SEQ ID NO: 25) |
| BVDV Template probe | ATCTGTTTCCGTCATCCTTAGTCCATTCCCATCATCGTCCA |
| (producing signal RND1 | GTTCTCTCTCCCTATAGTGAGTCGTATTACATAATChTGT |
| RNA)* ssDNA (artificial) | ATAAAAGTTCATTTGTGATCAACTCCATTTTTTx |
| | (SEQ ID NO: 26) |
| BVDV Template probe | TAGTATAAGTAAATCGCTTGCTGTATGTCGTTATTCTGCC |
| (producing signal B | GTAGGGCACCCTATAGTGAGTCGTATTACATAATChTGTA |
| RNA)* ssDNA (artificial) | TAAAAGTTCATTTGTGATCAACTCCATx (SEQ ID NO: 27) |
| BVDV Template probe | CTGGAACTGGATGGATGTCATTGCGTAAAGCCTCTATGCA |
| (producing signal 42_23 | CCTTATGGTGCCCTATAGTGAGTCGTATTACATAATChTG |
| RNA)* ssDNA (artificial) | TATAAAAGTTCATTTGTGATCAACTCCATx (SEQ ID NO: 28) |
| CBPP Extension Probe | GTCTCTATTGTTGTTAGAATCATCTGCTTGTGACGGATTATG |
| ssDNA (artificial) | (SEQ ID NO: 29) |
| CBPP Template Probe* | ATCTGTTTCCGTCATCCTTAGTCCATTCCCATCATCGTCCA |
| ssDNA (artificial) | GTTCTCTCTCCCTATAGTGAGTCGTATTACATAATChGTTT |
| | ATCTGAATGACTTGGCTTGTCAATTTTTTx (SEQ ID NO: 30) |
| CCPP Extension probe | GGAGATGTTAAAAATGTAGCATCAGTTGATTATG (SEQ ID |
| ssDNA (artificial) | NO: 31) |
| CCPP Template probe* | ATCTGTTTCCGTCATCCTTAGTCCATTCCCATCATCGTCCA |
| ssDNA (artificial) | GTTCTCTCTCCCTATAGTGAGTCGTATTACATAATChCTTT |
| | ATCTAGTGAAACTCCGAATTGTGAAx (SEQ ID NO: 32) |
| GAPDH Extension probe | GTTGGTGGTGCAGGAGGCATTGCTGACAATACTGATTATG |
| ssDNA (artificial) | (SEQ ID NO: 33) |
| GAPDH Template probe* | ATCTGTTTCCGTCATCCTTAGTCCATTCCCATCATCGTCCA |
| ssDNA (artificial) | GTTCTCTCTCCCTATAGTGAGTCGTATTACATAATChCTT |
| | GAGGGTGTTGTTATACTTCTCGTGGTTTTTTTTx (SEQ ID |
| | NO: 34) |
| Signal RNA (signal Ctrl) | GGGCAUGAAUAACGACAUACAGCAAGCGAUUUACUUAU |
| ssRNA (artificial) | ACUA (SEQ ID NO: 4) |
| Signal RNA (signal | GGGAGAGAGAACUGGACGAUGAUGGGAAUGGACUAAGG |
| RND1) ssRNA (artificial) | AUGACGGAAACAGAU (SEQ ID NO: 35) |
| Signal RNA (signal B) | GGGUGCCCUACGGCAGAAUAACGACAUACAGCAAGCGA |
| ssRNA (artificial) | UUUACUUAUACUA (SEQ ID NO:36) |
| Signal RNA (signal | GGGCACCAUAAGGUGCAUAGAGGCUUUACGCAAUGACA |
| 42_23) ssRNA (artificial) | UCCAUCCAGUUCCAG (SEQ ID NO: 37) |
| Signal DNA (signal | GGGAGAGAGAACTGGACGATGATGGGAATGGACTAAGG |
| RND1) ssDNA (artificial) | ATGACGGAAACAGAT (SEQ ID NO: 38) |

Amplification probe

| | |
|---|---|
| Amplification probe* | 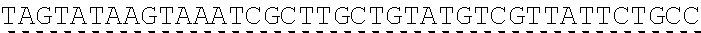 |
| ssDNA (artificial) | 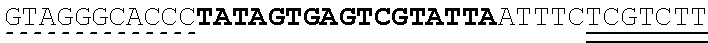 |
| | CChGGTCTCTCCTCTCAAGCCTCAGCGCTCTCTCTCCCx |
| | (SEQ ID NO: 3) |
| Amplification Probe P24* | 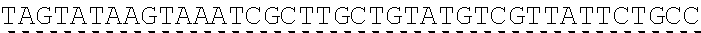 |
| ssDNA (artificial) | 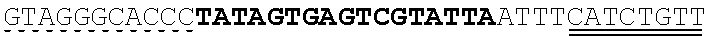 |
| | TChCGTCATCCTTAGTCCATTCCCATCATCGTCx |
| | (SEQ ID NO: 39) |
| Amplification Probe P80* | 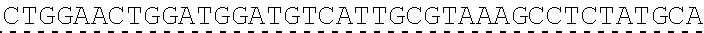 |
| ssDNA (artificial) | 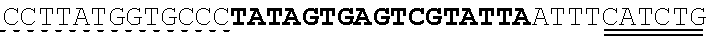 |
| | TTTChCGTCATCCTTAGTCCATTCCCATCATCGTCx |
| | (SEQ ID NO: 40) |

Toehold switch reporting

| | |
|---|---|
| Toehold switch 117 | GGGCCUUAGUCCAUUCCCAUCAUCGUCCAAGGCCUCUA |
| ssRNA (artificial) | GACAAUGAAACAGAGGAGAUGGACGAUGAUAAACCUGG |
| | CGGCAGCGCAAAAG (SEQ ID NO: 41) |
| Toehold switch 119 | GGGCCUUAGUCCAUUCCCAUCAUCGUCCAGUUCCUCUA |
| ssRNA (artificial) | ACGCCCAAUAACUAGAGGAGACGGACGAUGAUAAACCU |
| | GGCGGCAGCGCAAAAG (SEQ ID NO: 42) |
| Toehold switch 121 | GGGCCUUAGUCCAUUCCCAUCAUCGUCCAGUUCCUCUA |
| ssRNA (artificial) | ACAUGCCGCUAAACUAGAGGAGACGGACGAUGAUAAAC |
| | CUGGCGGCAGCGCAAAAG (SEQ ID NO: 43) |
| Toehold switch B | GGGAGAAAGUAAAUCGCUUGCUGUAUGUCGUUAAACAG |
| ssRNA (artificial) | AGGAGAUAACGAAUGACAGCAAGCAACCUGGCGGCAGC |
| | GCAAAAGAUGCGUAAA (SEQ ID NO: 5) |
| Toehold switch B version | GGGUAUAAGUAAAUCGCUUGCUGUAUGUCGUUAAACAG |
| 2, ssRNA (artificial) | AGGAGAUAACGAAUGACAGCAAGCAACCUGGCGGCAGC |
| | GCAAAAGAUGCGUAAA (SEQ ID NO: 44) |
| Toehold switch 42_23 | GGGAGAUAUGAACUGGAUGGAUGUCAUUGCGUAAAGCC |
| ssRNA (artificial) | UCUAUACCGAACGAAACAUAGAGGAGAUACGCAAUGAAA |
| | CGAUACAACCUGGCGGCAGCGCAAAAGCAAAGUAAG |
| | (SEQ ID NO: 45) |
| LacZ reporter | ATGACCATGATTACGGATTCACTGGCCGTCGTTTTACAAC |
| dsDNA | GTCGTGACTGGGAAAACCCTGGCGTTACCCAACTTAATC |
| | GCCTTGCAGCACATCCCCCTTTCGCCAGCTGGCGTAATA |
| | GCGAAGAGGCCCGCACCGATCGCCCTTCCCAACAGTTGC |
| | GCAGCCTGAATGGCGAATGGCGCTTTGCCTGGTTTCCGG |
| | CACCAGAAGCGGTGCCGGAAAGCTGGCTGGAGTGCGAT |
| | CTTCCTGAGGCCGATACTGTCGTCGTCCCCTCAAACTGG |
| | CAGATGCACGGTTACGATGCGCCCATCTACACCAACGTG |
| | ACCTATCCCATTACGGTCAATCCGCCGTTTGTTCCCACGG |
| | AGAATCCGACGGGTTGTTACTCGCTCACATTTAATGTTGA |
| | TGAAAGCTGGCTACAGGAAGGCCAGACGCGAATTATTTTT |
| | GATGGCGTTAACTCGGCGTTTCATCTGTGGTGCAACGGG |
| | CGCTGGGTCGGTTACGGCCAGGACAGTCGTTTGCCGTCT |
| | GAATTTGACCTGAGCGCATTTTTACGCGCCGGAGAAAAC |
| | CGCCTCGCGGTGATGGTGCTGCGCTGGAGTGACGGCAG |
| | TTATCTGGAAGATCAGGATATGTGGCGGATGAGCGGCAT |
| | TTTCCGTGACGTCTCGTTGCTGCATAAACCGACTACACAA |
| | ATCAGCGATTTCCATGTTGCCACTCGCTTTAATGATGATTT |
| | CAGCCGCGCTGTACTGGAGGCTGAAGTTCAGATGTGCGG |
| | CGAGTTGCGTGACTACCTACGGGTAACAGTTTCTTTATGG |
| | CAGGGTGAAACGCAGGTCGCCAGCGGCACCGCGCCTTT |
| | CGGCGGTGAAATTATCGATGAGCGTGGTGGTTATGCCGA |
| | TCGCGTCACACTACGTCTGAACGTCGAAAACCCGAAACT |
| | GTGGAGCGCCGAAATCCCGAATCTCTATCGTGCGGTGGT |
| | TGAACTGCACACCGCCGACGGCACGCTGATTGAAGCAGA |
| | AGCCTGCGATGTCGGTTTCCGCGAGGTGCGGATTGAAAA |
| | TGGTCTGCTGCTGCTGAACGGCAAGCCGTTGCTGATTCG |
| | AGGCGTTAACCGTCACGAGCATCATCCTCTGCATGGTCA |
| | GGTCATGGATGAGCAGACGATGGTGCAGGATATCCTGCT |
| | GATGAAGCAGAACAACTTTAACGCCGTGCGCTGTTCGCA |
| | TTATCCGAACCATCCGCTGTGGTACACGCTGTGCGACCG |
| | CTACGGCCTGTATGTGGTGGATGAAGCCAATATTGAAAC |
| | CCACGGCATGGTGCCAATGAATCGTCTGACCGATGATCC |
| | GCGCTGGCTACCGGCGATGAGCGAACGCGTAACGCGAA |
| | TGGTGCAGCGCGATCGTAATCACCCGAGTGTGATCATCT |
| | GGTCGCTGGGGAATGAATCAGGCCACGGCGCTAATCAC |
| | GACGCGCTGTATCGCTGGATCAAATCTGTCGATCCTTCCC |
| | GCCCGGTGCAGTATGAAGGCGGCGGAGCCGACACCACG |
| | GCCACCGATATTATTTGCCCGATGTACGCGCGCGTGGAT |
| | GAAGACCAGCCCTTCCCGGCTGTGCCGAAATGGTCCATC |
| | AAAAAATGGCTTTCGCTACCTGGAGAGACGCGCCCGCTG |
| | ATCCTTTGCGAATACGCCCACGCGATGGGTAACAGTCTT |
| | GGCGGTTTCGCTAAATACTGGCAGGCGTTTCGTCAGTAT |
| | CCCCGTTTACAGGGCGGCTTCGTCTGGGACTGGGTGGAT |
| | CAGTCGCTGATTAAATATGATGAAAACGGCAACCCGTGGT |
| | CGGCTTACGGCGGTGATTTTGGCGATACGCCGAACGATC |
| | GCCAGTTCTGTATGAACGGTCTGGTCTTTGCCGACCGCA |
| | CGCCGCATCCAGCGCTGACGGAAGCAAAACACCAGCAG |
| | CAGTTTTTCCAGTTCCGTTTATCCGGGCAAACCATCGAAG |
| | TGACCAGCGAATACCTGTTCCGTCATAGCGATAACGAGC |
| | TCCTGCACTGGATGGTGGCGCTGGATGGTAAGCCGCTG |
| | GCAAGCGGTGAAGTGCCTCTGGATGTCGCTCCACAAGGT |
| | AAACAGTTGATTGAACTGCCTGAACTACCGCAGCCGGAG |
| | AGCGCCGGGCAACTCTGGCTCACAGTACGCGTAGTGCAA |
| | CCGAACGCGACCGCATGGTCAGAAGCCGGGCACATCAG |
| | CGCCTGGCAGCAGTGGCGTCTGGCGGAAAACCTCAGTG |
| | TGACGCTCCCCGCCGCGTCCCACGCCATCCCGCATCTGA |
| | CCACCAGCGAAATGGATTTTTGCATCGAGCTGGGTAATAA |
| | GCGTTGGCAATTTAACCGCCAGTCAGGCTTTCTTTCACAG |
| | ATGTGGATTGGCGATAAAAAACAACTGCTGACGCCGCTG |
| | CGCGATCAGTTCACCCGTGCACCGCTGGATAACGACATT |
| | GGCGTAAGTGAAGCGACCCGCATTGACCCTAACGCCTGG |
| | GTCGAACGCTGGAAGGCGGCGGGCCATTACCAGGCCGA |
| | AGCAGCGTTGTTGCAGTGCACGGCAGATACACTTGCTGA |
| | TGCGGTGCTGATTACGACCGCTCACGCGTGGCAGCATCA |
| | GGGGAAAACCTTATTTATCAGCCGGAAAACCTACCGGATT |
| | GATGGTAGTGGTCAAATGGCGATTACCGTTGATGTTGAA |
| | GTGGCGAGCGATACACCGCATCCGGCGCGGATTGGCCT |
| | GAACTGCCAGCTGGCGCAGGTAGCAGAGCGGGTAAACT |
| | GGCTCGGATTAGGGCCGCAAGAAAACTATCCCGACCGCC |
| | TTACTGCCGCCTGTTTTGACCGCTGGGATCTGCCATTGTC |
| | AGACATGTATACCCCGTACGTCTTCCCGAGCGAAAACGG |
| | TCTGCGCTGCGGGACGCGCGAATTGAATTATGGCCCACA |
| | CCAGTGGCGCGGCGACTTCCAGTTCAACATCAGCCGCTA |
| | CAGTCAACAGCAACTGATGGAAACCAGCCATCGCCATCT |
| | GCTGCACGCGGAAGAAGGCACATGGCTGAATATCGACG |
| | GTTTCCATATGGGGATTGGTGGCGACGACTCCTGGAGCC |
| | CGTCAGTATCGGCGGAATTCCAGCTGAGCGCCGGTCGCT |
| | ACCATTACCAGTTGGTCTGGTGTCAAAAATAA |
| | (SEQ ID NO: 6) |

RT-qPCR primers

| | |
|---|---|
| Signal B RT-qPCR | GGTGCCCTACGGCAG (SEQ ID NO: 7) |
| Forward Primer | |
| ssDNA (artificial) | |
| Signal B RT-qPCR | TAGTATAAGTAAATCGCTTGCTGTATGTCG (SEQ ID NO: 8) |
| Reverse Primer | |
| ssDNA (artificial) | |
| RND1 RT-qPCR | GGGAGAGAGAACTGGACGATGA (SEQ ID NO: 46) |
| Forward Primer | |
| ssDNA (artificial) | |
| RND1 RT-qPCR | ATCTGTTTCCGTCATCCTTAGTCCATTC (SEQ ID NO: 47) |
| Reverse Primer | |
| ssDNA (artificial) | |
| 42_23 RT-qPCR | CACCATAAGGTGCATAGAGGCTT (SEQ ID NO: 48) |
| Forward Primer | |
| ssDNA (artificial) | |
| 42_23 RT-qPCR | CTGGAACTGGATGGATGTCATTGC (SEQ ID NO: 49) |
| Reverse Primer | |
| ssDNA (artificial) | |

PCR primers

| | |
|---|---|
| PCR Forward Primer | GAAGTCTAACGCTGCTCTGG (SEQ ID NO: 9) |
| (S19) (artificial) | |
| PCR Reverse Primer | TCTCAAATGCCTGAGGTTTCAG (SEQ ID NO: 10) |
| (S20) (artificial) | |

*h refers to hexaethylenglycol (HEG) moiety
*x refers to 3′ phosphorylation
All sequences are presented in 5′ → 3′ orientation
dsDNA = double-stranded deoxyribonucleic acid
ssDNA = single-stranded deoxyribonucleic acid
dsRNA = double-stranded ribonucleic acid
ssRNA = single-stranded ribonucleic acid

In the extension probe design, the underlined region is complementary to target.

In the template probe design, the underlined region is complementary to target; the double underlined region is the template for signal RNA1; the region in bold is the T7 promoter.

In the amplification probe design, the double underlined is the region complementary to signal RNA1; the T7 promoter is in bold; the dashed underline sequence is the template for signal RNA2.

Toehold switches are presented in their RNA form, after transcription by T7 RNA polymerase from a dsDNA template containing a T7 promoter in a cell-free reaction. The coding sequence of the reporter protein lacZ is indicated separately as (SEQ ID NO:6). The coding sequence of the lacZ protein was added immediately after the presented toehold switch RNA sequences.

The toehold switch referred to as Toehold switch B version 2 (and its activation trigger referred to as Signal B) is identified as Toehold switch B in Pardee, et al., (2014, ibid). The toehold switches referred to as 117, 119, 121 (all activated by signal RN D1) and 42_23 (activated by signal 42_23) were designed by Biotangents Ltd.

The invention was exemplified by combining detection and amplification modules, encompassed by the SMART reaction, with a reporting module containing either toehold switch B (Pardee et al. 2014, ibid), toehold switch 42_23 or toehold switch 121, the latter two designed by Biotangents Ltd. The reactions were performed using synthetic nucleic acids targets in either three or two pipetting stages. Experimental data on the performance of the technology in the presence of additional nucleic acids extracted from cattle blood is also included in the examples. Additionally, experiments evaluating the performance of different DNA polymerases, signal RNA sequences and toehold switch designs are also presented.

1.2. Protocol for Assessing SMART Reaction Activity

The following describes the methodology for setting up a SMART reaction for producing signal RNA, the mechanism of which is explained in FIG. 2. Note that the data in Section 1.7.1 has been generated using the first protocol listed: subsequent optimisation of the protocol has resulted in the protocol used for Sections 1.7.2 to 1.7.6, which is listed here separately.

(For Section 1.7.1 Only)

All samples were set up in triplicates, reagents were thawed on ice prior to use, and work was carried out in a dedicated RNase-free area. All incubations took place in a thermocycler at 41° C. with a 105° C. heated lid. The SMART reaction was set up by adding 25 fmol target RNA (SEQ ID NO:1) (Hall et al., 2002, ibid) to a mixture containing 0.87× RNAPol Reaction buffer (B9012S, New England Biolabs), 1.53 U/μl NEB Murine RNase Inhibitor (M0314, New England Biolabs), 0.382 nM extension probe (SEQ ID NO:20) (Hall et al., 2002, ibid) 0.076 nM template probe (SEQ ID NO:2) (Hall et al., 2002, ibid) and nuclease-free water to a total volume of 1.64 μl. A negative control was set up for each sample with the same components but no target RNA. Probes were annealed to the target RNA by incubating for 30 min. After that, 2.99 μl of a solution containing 250 fmol amplification probe, 0.692× RNA Polymerase Reaction buffer (B9012S, New England Biolabs), 23.64 μM dNTP solution mix (N0447, New England Biolabs), 1.19 mM rNTP solution mix (N0466, New England Biolabs), 0.216 U/μl Bst 3.0 (M0374, New England Biolabs), 5.41 U/μl T7 RNA Polymerase (M0251, New England Biolabs) and nuclease-free water was added to each sample. The samples were then incubated for 3.5 h to allow the production of signal RNA. As a final step, 0.37 μl nuclease-free water was added to the samples to obtain a total volume of 5 μl.

(For Sections 1.7.2 to 1.7.6)

All samples were set up in triplicate, reagents were thawed on ice prior to use, and work was carried out in a dedicated RNase-free area. All incubations took place in a thermocycler at 41° C. with a 105° C. heated lid. The SMART reaction was set up by adding target RNA at the indicated concentration to a mixture containing 74.5% PURExpress® Solution A (E6800, New England Biolabs), 0.43× Thermopol Reaction buffer (B9004, New England Biolabs), 1.53 U/μl NEB Murine RNase Inhibitor (40 U/μl), 0.382 nM of the indicated extension probe, 0.076 nM of the indicated template probe and nuclease-free water to a total volume of 1.64 μl. An exception to this is in Section 1.7.2 (FIG. 8A), where SMART reactions were set up by adding 25 fmol target RNA to a mixture containing either 0.43× RNA Polymerase

Reaction buffer (B9012S, New England Biolabs), 0.43× Isothermal Amplification Buffer for BST 2.0 only (B0537, New England Biolabs), 0.43× Isothermal Amplification Buffer II for BST.3.0 only (B0374, New England Biolabs) or 0.43× Thermopol Reaction Buffer for Vent® and DeepVent® only (B9004, New England Biolabs). A negative control was set up for each sample with the same components but without target RNA. Probes were annealed to the target RNA by incubating for 30 min. After that, 2.99 μl of a solution containing 250 fmol amplification probe (added where indicated; otherwise omitted from the reaction), 0.35× RNAPol Reaction Buffer (B9012, New England Biolabs), 0.5× Thermopol Reaction buffer (B9004, New England Biolabs), 23.64 μM dNTP solution mix, 1.19 mM rNTP solution mix, 0.22 U/μl of the indicated DNA polymerase(s), 5.41 U/μl T7 RNA Polymerase and nuclease-free water was added to each sample. The samples were then incubated for 2 h to allow the production of signal RNA. During DNA polymerase evaluation, the standard enzyme used was BST 3.0 (M0374, New England Biolabs). This was compared to BST 2.0 (M0537, New England Biolabs), Vent® exo- (M0257, New England Biolabs), Therminator (M0261, New England Biolabs), Deep Vent® exo⁻ (M0259, New England Biolabs), and Klenow exo⁻ (M0212, New England Biolabs) enzymes.

1.3 Protocol for Signal RNA Detection by Reverse Transcription Quantitative PCR (RT-qPCR)

1.3.1 Treatment with DNasel

3 μl of each sample was used in RT-qPCR reactions to quantify signal RNA production. Samples were treated with DNA-free™ DNA Removal Kit (AM1906, ThermoFisher Scientific) to remove DNA probes from the reaction. 1.4 μl 10' DNAse I Buffer, 1 μl rDNAse I and 8.6 μl nuclease-free water were added to each sample, followed by a 1 h incubation at 37° C. in a thermocycler with a 105 ° C. heated lid. After that, 2 pl of resuspended DNase inactivation Reagent was added and samples were incubated for 2 min at room temperature, with mixing (vortexing) 3 times during the incubation. Samples were then centrifuged at 10,000×g for 1.5 min and 10 μl of the supernatant was transferred to a new tube without disrupting the pellet. All samples were diluted 1 in 100 prior to RT-qPCR.

1.3.1 Treatment with Exonuclease V (RecBCD)

Samples were treated with Exonuclease V (RecBCD) (M0345, New England Biolabs) to remove DNA probes prior to the quantification of signal RNA by RT-qPCR. 1 μl 10× NEBuffer 4 (B7004, New England Biolabs), 1 μl 10 mM ATP (P0756, New England Biolabs), 0.33 μl Exonuclease V (10,000 U/ml) and 6.87 pl nuclease-free water were added to 0.80 μl of a 1 in 400 dilution (in nuclease-free water) of each sample. The reactions were incubated for 30 min at 37° C. in a thermocycler with a 105° C. heated lid, followed by an incubation for 30 min at 70° C. in a thermocycler with a 105° C. heated lid to inactivate the Exonuclease V.

1.3.2 RT-qPCR on AB17500 Thermal Cycler

The following reagents were mixed in a 96-well plate: 1× Luna Universal One-Step Reaction Mix (M3003, New England Biolabs), 1× Luna Warm Start RT Enzyme Mix (M3003, New England Biolabs), 0.4 μM RT-qPCR forward primer, 0.4 μM RT-qPCR reverse primer, 3.7 μl (for diluted DNAsel-treated samples) or 2 82 l (for diluted Exonuclease V-treated samples) of SMART reaction samples and RNAse-free water to 10 μl. A 4-log standard curve was generated by adding 1-1000 pg of in vitro transcribed or synthetic signal RNA in 2 μl nuclease-free water to RT-qPCR reactions instead of SMART reaction samples.

RT-qPCR reactions were performed using an ABI 7500 Real Time Cycler (Applied Biosystems™) with the following cycling parameters:
10 min at 55° C.,
1 min at 95° C.,
40 cycles of 10 s at 95° C. and 1 min at 60° C.

Following cycling, a melt curve analysis was performed by initially heating to 95° C. for 15 s before cooling to 60° C. for 1 min, followed by ramping the temperature to 95° C. at a rate of 0.66° C./min.

1.3.3 Results Analysis

Results were analysed with Applied Biosystems 7500 Software v2.3. For section 1.7.1, The estimated normalised yield (pg/μl) of signal RNA present in the samples after SMART amplification was estimated using the formula described below, where E=Enzymes, B=Buffer, P=Probes, T=Target RNA. The average of triplicates was calculated±standard deviation.

$$Normalised quantity ( pg ) = SMART target ( E + B + P + T ) quantity mean ( pg ) - SMART negative control II ( E + B + P ) quantity mean ( pg )$$

$$Estimated yield ( pg \text{/} µl ) = \frac{Normalised quantity ( pg )}{3.7 ( µl )} * 100 * 5.33$$

Where 3.7 is the volume of the diluted DNase treated sample added to the PCR, 100 is the dilution factor after DNase I treatment and 5.33 is the dilution factor at the DNase I treatment.

For sections 1.7.2 onwards, the estimated yield (pg/pl) of signal RNA present in the SMART samples after amplification was estimated using the formula described below. The average of triplicates was calculated±standard deviation. This was performed on samples with target and samples without target.

0.4 μl of the SMART reaction (total volume=4.625 μl) was initially diluted to 160 μl. From this, 0.8 μl was added to the Exonuclease V reaction (total volume=10 μl), and 2 μl of the digestion was added to the RT-qPCR. The equation is therefore as follows:

$$Signal {RNA}_{SMART assay} ( pg ) = Signal {RNA}_{RT \text{-} qPCR} ( pg ) * \frac{10}{2} * \frac{160}{0.8} * \frac{4.625}{0.4}$$

The signal-to-noise ratio was calculated by dividing the total signal RNA yield (pg) in the presence of the target RNA by the total signal RNA yield (pg) in the absence of the target RNA.

$$Signal \text{:} noise ratio = \frac{Signal {RNA}_{target RNA present}}{Signal {RNA}_{target RNA absent}}$$

1.4 Protocol for Assessing Toehold Switch Activity

1.4.1 Toehold Switch Construction

Toehold switch sequences were synthesised as gene fragments (Integrated DNA Technologies) and inserted into a plasmid with the pCOLADuet™-1 (Zverev & Khmel, Plasmid 14:192-199, 1985; 71406, EMD Millipore) backbone containing a lacZ reporter gene. For Toehold Switch B (SEQ ID NO:5), this insertion was performed using Gibson Assembly using the NEBuilder HiFi DNA Assembly Master Mix (E2621, New England Biolabs).

Other toehold switches (42_23, 117, 119 and 121) were synthesized as dsDNA gene fragments (IDT), digested with BspQl endonuclease (R0712, New England Biolabs) and ligated with T4 DNA Ligase (M0202, New England Biolabs) into the BspQl restriction sites of pCOLADuet™-1, which contained the lacZ reporter gene.

The plasmid assembly product was transformed into NEB® Stable Competent E coli (High Efficiency) (C3040, New England Biolabs). Plasmid sequences were verified by DNA sequencing (Eurofins Genomics). Double-stranded DNA containing the T7 promoter, toehold switch and lacZ reporter gene, for use in a cell-free reaction, was amplified from the resultant plasmid by PCR (Q5 High-Fidelity 2× Master Mix, NEB) with primers SEQ ID NO:9 and SEQ ID NO:10. The amplified product was then column purified using the Monarch® PCR & DNA Cleanup Kit (T1030, New England Biolabs).

1.4.2 Support Medium Preparation

Filter paper (1442-042, Whatman) was blocked with 5% w/v Bovine Serum Albumin solution (B4287, New England Biolabs) to reduce reagent adsorption to cellulose fibres. Blocking was performed overnight by incubating filter papers in the solution at 4° C. with 80 rpm orbital shaking (New Brunswick Scientific G-25 Incubator Shaker). The blocked filter paper was allowed to dry at room temperature for 3 days and was then cut into smaller O 2 mm paper discs using a Stiefel Sterile Biopsy Punch 2.0 mm (D5242, Williams Medical). Paper discs were placed into a black-walled, clear-and-flat-bottomed 384-well plate (3542, Corning).

1.4.3 Preparation of Cell-Free Reactions on Paper

Cell-free reactions were performed using the PURExpress® In Vitro Protein Synthesis Kit (E6800, New England Biolabs). Reactions were prepared in a total volume of 1.8 μl and pipetted onto 2.0 mm paper discs, blocked with Bovine Serum Albumin (BSA) as per Pardee et al., 2014 ibid. Each paper disc contained 1.26 μl of cell-free system (57% Solution A and 43% Solution B), 0.8 U/μl of Murine RNase Inhibitor, 0.6 mg/ml of chlorophenol red-β-D-galactopyranoside substrate (10884308001, Roche), 12.28 nM of Toehold Switch B and nuclease-free water. Reaction components were then flash frozen on dry ice and lyophilised for 22 h. After this, paper discs were rehydrated with 1.8 μl of either undiluted SMART reaction or nuclease-free water (as a negative control).

1.4.4 Preparation of Cell-Free Reactions in Solution

Cell-free reactions were performed using the PURExpress® In Vitro Protein Synthesis Kit. Reactions were prepared in a total volume of 5 μl. Each reaction contained 40% v/v PURExpress® Solution A, 30% v/v PURExpress® Solution B, 0.8 U/μl of Murine RNase Inhibitor, 0.6 mg/ml chlorophenol red-β-D-galactopyranoside substrate, 2.24 nM (except where indicated otherwise) of the indicated toehold switch, and either nuclease-free water with a specified concentration of signal RNA or 1.4 μl of SMART reaction sample.

1.4.5 Quantification of Toehold Switch Activation

The yellow-to-purple colour change of the cell-free reaction solution, containing the toehold switch-lacZ DNA cassette, indicative of the conversion of chlorophenol red-β-D-galactopyranoside substrate by LacZ to chlorophenol red, was monitored using a GloMax®-Multi+Microplate Multimode Reader (Promega). Samples was incubated for 5 h at 41° C. with absorbance measurements at 560 nm or 600 nm taken every 3 min.

1.4.6 Results Analysis of Cell-Free Reactions

The average background signal from blank samples (containing all reaction components except for the toehold switch or signal RNA) was subtracted from the measurements of the remaining test samples. Absorbance results of the cell-free reactions containing SMART target samples (E+B+P+T) were normalised against the results of its negative control (reactions with SMART negative control (E+B+P) samples), where E=Enzymes, B=Buffer, P=Probes, T=Target RNA. The normalised results were analysed by t-test (two-tailed distribution, two-sample equal variance) to determine if their absorbances were significantly different at each timepoint.

The time to result was determined to be the time taken for the measured absorbance to be equal or greater than 1.56 absorbance units, which had previously been determined to be the absorbance corresponding to a visible colour change in the cell-free assay. The signal-to-noise ratio was calculated by firstly measuring the absorbance in the negative control reaction at the time at which the test sample reached an absorbance of 1.56 absorbance units and converting the raw absorbance into % substrate conversion using a set of standards prepared by mixing known amounts of chlorophenol red-β-D-galactopyranoside substrate and chlorophenol red together in a 5 μl reaction volume, and measuring the resulting absorbance at 560 nm. The percentage conversion at an absorbance of 1.56 absorbance units was calculated to be 20.9%. Therefore, the signal-to-noise ratio was:

$$Signal \text{:} noise ratio = \frac{20.9}{\% substrate conversion in negative control at time to result}$$

1.5 Performance of a Two-Step Assay

All samples were set up in triplicate, reagents were thawed on ice prior to use and work was carried out in a dedicated RNase-free area. To prevent potential cross-contamination, negative reaction components were set up in a different laboratory area to positive reaction components. All incubations took place in a thermocycler at 41° C. a 105° C. heated lid. SMART reaction samples were prepared by adding 153 pM of target RNA to a mixture containing 26.3% v/v PURExpress® Solution A, 0.5× Thermopol Reaction Buffer, 0.35× RNA Polymerase Reaction Buffer, 1.27 U/μl NEB Murine RNase Inhibitor (40 U/μl), 23.64 μM dNTPs, 1.19 mM rNTPs, 5.41 U/μl T7 RNA Polymerase, 13.51 nM extension probe, 7.47 nM template probe, 2.7 nM toehold switch PCR product and nuclease-free water to a total volume of 4.63 μl. A negative control was set up for each sample with the same components but no target RNA. The samples were then incubated for 2 h to allow the generation of signal RNA and toehold switch-lacZ mRNA.

Following incubation, 1.5 μl of sample was transferred to a cell-free reaction containing 40% v/v PURExpress® Solution A, 30% v/v PURExpress® Solution B, 0.8 U/μl of Murine RNase Inhibitor, and 0.6 mg/ml of chlorophenol red-β-D-galactopyranoside substrate in a final volume of 5 μl. Absorbance was then measured as per Section 1.4.5.

1.6 Use of DNA Rather than RNA to Produce Signal RNA2.

All samples were set up in triplicate, reagents were thawed on ice prior to use, and work was carried out in a dedicated RNase-free area. All incubations took place in a thermocycler at 41° C. with a heated lid at 105° C. The amplification reaction was set up by adding signal RNA (SEQ ID NO:35) or DNA (SEQ ID NO:38) at the indicated concentrations to a mixture containing 250 fmol amplification probe P24 (SEQ ID NO:39), 74.5% v/v PURExpress® Solution A, 0.35× RNAPol Reaction Buffer, 0.5× Thermopol Reaction buffer, 23.64 μM dNTP solution mix, 1.19 mM rNTP solution mix, 0.22 U/μl of Therminator DNA polymerase, 5.41 U/μl T7 RNA Polymerase and nuclease-free water to a total 4.63 μl reaction volume. The samples were then incubated for 2 h to allow the production of signal B RNA (SEQ ID NO:36).

1.7 Results

1.7.1 Combination of Published SMART and Toehold Switch Sequences

SMART target sample contained Enzymes (E), Buffers (B), Probes (P) and Target RNA (T). Two negative controls contained 1) only Enzymes (E) and Buffers (B) or 2) Enzymes (E), Buffers (B) and Probes (P) but no target RNA. The target RNA used was SEQ ID NO:1 and the probes used were SEQ ID NO:2, SEQ ID NO:3 and SEQ ID NO:20. Upon completion, each SMART reaction sample was divided into two parts:
3 μl was treated with DNasel. RNA signal was quantified by RT-qPCR using primers (SEQ ID NO:7) and (SEQ ID NO:8) (FIG. 6)
2 μl was combined with toehold switch B (SEQ ID NO:5), fixed on paper (FIG. 7)

In the presence of RNA target, significantly more signal RNA was produced than in the absence of target (p-value<0.001). An estimated concentration of RNA signal (noise subtracted) was 113±21 ng μl⁻¹ (8.3±1.5 μM) (FIG. 6).

After quantifying signal RNA production using RT-qPCR, toehold switches fixed onto paper were rehydrated with the remaining SMART reaction sample volume and incubated at 41° C. Samples containing the target of the SMART reaction started to develop colour and were significantly different (p<0.05) from SMART negative control I (E+B) samples after 42 min of incubation. Target samples also showed significant differences (p<0.01) in absorbance from SMART negative control II (E+B+P) throughout the incubation period and developed a clear difference in colour, with negative control samples remaining yellow and target samples turning purple during the incubation (FIG. 7A). The maximum rate of change in β-galactosidase enzyme production in samples containing the SMART reaction target was 20±2 times higher than in samples rehydrated with the SMART negative control II (E+B+P) sample and occurred at approximately 54 min of incubation (FIG. 7B). These results indicate that the target detection and signal generation modules are functional when combined and can produce a straightforward visual response upon detection of specific RNA sequences.

1.7.2 Use of Alternative DNA Polymerases

Several different DNA polymerases were evaluated to determine which enzyme functions most efficiently in the SMART reaction. The enzymes evaluated differed in a range of properties including thermal stability, helicase activity and exonuclease activity and were compared against 0.22 U/μl Bst 3.0. Bst 2.0, Vent® and Klenow DNA polymerases were tested at 0.22 U/μl in a SMART reaction designed to detect a BVDV target RNA (SEQ ID NO:21) using probes SEQ ID NO:23 and SEQ ID NO:25. The production of Signal RNA1 (SEQ ID NO:35) was assessed by RT-qPCR (FIG. 8A). This data allowed us to select Vent® DNA polymerase, which showed approximately 20× greater signal-to-noise ratio than Bst 3.0.

One possible reason for Vent® DNA polymerase performing better than the other enzymes tested may be the presence of high concentrations of ribonucleotides (rNTPs) in the assay, which are required for the production gene transcripts. It has been reported (McCallum & Chaput, Chemcomm, 20:2938-2940, 2009) that Vent® can incorporate rNTPs in place of dNTPs, whereas other DNA polymerases may be inhibited by high rNTP concentrations. Use of Vent® in the SMART assay could result in the efficient production of an extended dsDNA-RNA hybrid molecule that would act as a template for T7 RNA polymerase.

Having identified this possible mechanism of action, we investigated alternative enzymes with similar characteristics to Vent® DNA polymerase. McCallum & Chaput (2009, idib) identified Therminator DNA polymerase as also being tolerant to high concentrations of rNTPs and able to incorporate rNTPs in place of dNTPs. We compared Therminator to Vent® DNA polymerase in the SMART reaction (FIG. 8B). The performance of the two enzymes was compared when 0.22 U/μl of each was added in separate reactions and when 0.22 U/μl of Vent® and 0.22 U/μl of Therminator were added in combination to the same reaction. This experiment demonstrated that using Therminator resulted in the largest quantity of RNA signal (approximately 2× more than Vent®) and the highest signal-to-noise ratio (approximately 1.5× greater than Vent®), both in the presence and absence of Vent®.

The use of Therminator is a novel non-obvious change to the published assay: Therminator is commercially advertised as being used for incorporation of nucleotides with large additional moieties, such as fluorophores (Hori et al., Bioorg Med Chem Lett., 24:2134-2136, 2004). Therminator is the A485L mutant of parent DNA polymerase 9° N polymerase (Gardner & Jack, NAR, 30:605-613, 2002). When 0.22 U/μl of 9° N polymerase was tested in the SMART reaction, the signal-to-noise ratio and the amount of signal RNA1 target produced were observed to be lower than when Therminator is used (data not shown), meaning that the specific use of Therminator in the assay with coupled DNA extension and transcription is important to get the levels of signal observed.

1.7.3 Production of Different Signal RNA1 Sequences by SMART

It was investigated whether the Signal RNA1 sequence itself influences Signal RNA1 yields. This was done by testing, using the SMART assay, three template probes (SEQ ID NO:26), (SEQ ID NO:27) and (SEQ ID NO:28) that differed only in the Signal RNA1 sequence. Each of these probes was designed to detect the BVDV target (SEQ ID NO:21) and was tested in combination with extension probe (SEQ ID NO:25) and Therminator DNA polymerase.

FIG. 9 shows the effect of the three template probe designs on Signal RNA1 production. Use of P23 (SEQ ID NO:26) (which produces signal RNA RND1 (SEQ ID NO:35)) led to a similar amount of signal RNA (1.1 μM) as P130 (SEQ ID NO:27) (which produces signal RNA trigger B (SEQ ID NO:36)), whereas P131 (SEQ ID NO:28) (which produces signal RNA 42_23 (SEQ ID NO:37)) produced much less signal RNA (0.14 μM). However, the use of P23 resulted in a 2.8× greater signal-to-noise ratio than P130, due to the use of P130 resulting in greater signal RNA1 generation in the absence of the target RNA. Therefore, we concluded that the Signal RNA1 sequence (SEQ ID NO:35) produced from template probe P23 was the best design of the three designs tested.

1.7.4 Use of Alternative Toehold Switch Designs

The toehold switch is activated by the Signal RNA to allow translation of the reporter sequence (e.g. lacZ, SEQ ID NO:6). We have investigated the impact of the toehold switch sequence on the colour-change reaction that is mediated by lacZ and the functionality of toehold switches with an RBS positioned in the stem rather than in the loop, the latter being indicated in prior art (Pardee et al. 2014, ibid). Toehold switch designs tested were 117 (SEQ ID NO:41), 119 (SEQ ID NO:42), 121 (SEQ ID NO:43), switch B (SEQ ID NO:5), B version 2 (SEQ ID NO:44), and 42_23 (SEQ ID NO:45). Toehold switches B and B version 2 had the RBS positioned in the loop of the switch, while toehold switches 42_23, 117, 119 and 121 had the RBS positioned in the stem of the switch. We found that the signal-to-noise ratio and time to result (defined as the time-point at which the A560 value indicates that a clear colour-change, visible to the naked eye, has occurred) varied considerably depending on the toehold switch sequence (FIG. 10A). 42_23 gave the fastest time to result (40.5 min) but a low signal-to-noise ratio (5.2), whereas TS121 had the longest time to result (119 min) but had the highest signal-to-noise ratio (32.2). Noise in this context refers to the production of the purple chlorophenol red in the absence of the Signal RNA. Therefore, the properties of toehold switches can vary: depending on the requirements of a particular application, a toehold switch could be chosen to have a quicker time to result or a higher signal-to-noise ratio.

In addition to the toehold switch design, the impact of altering the concentration of the toehold switch DNA cassette (which is transcribed by a cell-free transcription/translation system to produce the functional toehold switch-containing RNA transcript) was investigated (FIG. 10B). Our results have demonstrated that increasing the toehold switch concentration can shorten the time to result considerably without affecting the signal-to-noise ratio.

1.7.5 Sensitivity and Specificity

In order to test the robustness of the assay, 12 positive (each containing 16.6 pM of CCPP target RNA sequence (SEQ ID NO:23)) and 12 negative replicates of the assay were tested using the SMART reaction (with Therminator DNA polymerase and probes (SEQ ID NO:31) and (SEQ ID NO:32)) and a CCPP target RNA sequence (SEQ ID NO:23) concentration of 153 pM. To these reactions, 1.39 pl of nucleic acid extracts (extracted from 250 μl of bovine plasma using a QIAamp® viral RNA mini kit (52904, Qiagen)) from healthy bovine blood samples (EDTA whole blood, plasma or buffy coat fractions) were added to evaluate assay specificity in the presence of an excess of non-target nucleic acids. Total nucleic acid concentration of the extracts ranged from 118.4 ng/pl to 159.8 ng/pl. Following incubation, SMART reaction samples were treated with Exonuclease V and analysed by RT-qPCR using primers SEQ ID NO: 46 and SEQ ID NO:47 to evaluate the production of signal RNA1 (SEQ ID NO:35) in the presence and absence of the target RNA.

This test produced a highly reproducible quantity of signal RNA1 (SEQ ID NO:35) (883 nM in the presence of target compared to only 34 nM in the absence of target, giving a signal-to-noise ratio of 26) and a robust colour-change that was visible to the naked eye was observed once the samples were combined with the cell-free reaction containing toehold switch 42_23 (SEQ ID NO:45) (FIG. 12). Importantly, no false-positive signals were observed, demonstrating that the assay is highly specific and does not cross-react with other RNA or DNA species that are likely to be present in nucleic acid samples from blood plasma.

1.7.6 Additional Targets Tested

In addition to detecting target nucleic acid corresponding to BVDV and CCPP, which have been demonstrated above, we have also demonstrated the ability to produce signal RNA1 using 153 pM of a target RNA sequence (SEQ ID NO:22) corresponding to Mycoplasma mycoides subsp. mycoides, the pathogenic organism that causes Contagious Bovine Pleuropneumonia (CBPP) (FIG. 13A). This was detected by performing the SMART assay with probes (SEQ ID NO:29) and (SEQ ID NO:30) and Therminator DNA polymerase. Samples were then treated with Exonuclease V and analysed by RT-qPCR with primers (SEQ ID NO:46) and (SEQ ID NO:47). This resulted in the production of 145 nM of signal RNA1 in the presence of the target RNA, and 20.5 nM in the absence of the target RNA, giving a signal-to-noise ratio of 7.1.

In addition, we have demonstrated detection of 15.3 nM of a sequence corresponding to mammalian glyceraldehyde 3-phosphate dehydrogenase (GAPDH) (SEQ ID NO:24) (FIG. 13B) as targets for SMART, as described in the methods section with the exception that a 56% v/v final concentration of PURExpress® Solution A was added at the annealing step. 0.22 U/μl of both Therminator and Vent® DNA polymerase and probes (SEQ ID NO:33), (SEQ ID NO:34) and (SEQ ID NO:40) were used. Following the SMART reaction, samples were treated with Exonuclease V and analysed by RT-qPCR with primers (SEQ ID NO:48) and (SEQ ID NO:49). This demonstrated the production of 490 nM of signal RNA2 (SEQ ID NO:45) in the presence of target RNA and only 41 nM in the absence of target RNA.

Instead of being linked to a pathogenic organism, GAPDH is an endogenous host housekeeping gene. The detection of GAPDH can be used as a positive control in a diagnostic reaction. As this target is highly conserved across species, the Moduleic Sensing ^{TM} probes have been designed to allow us to detect GAPDH from multiple species (e.g. bovine and caprine), meaning that this assay design can be used as a positive control for multiple targets from multiple species.

1.7.7 Two-Step Assay

To further simplify the assay, performing the assay as a two-step reaction was trialled. This has not previously been reported in the literature. The approach combined 1) the annealing of probes to the target RNA with 2) the DNA extension and transcription steps in a single step. This was done by adding the template (SEQ ID NO:31) and extension probes (SEQ ID NO:32) and the CCPP target RNA (SEQ ID NO:23) directly to the SMART reaction components, allowing both annealing and amplification (with Therminator DNA polymerase) to occur concurrently in the same reaction tube. In addition, the toehold switch 121 (SEQ ID NO:43) DNA cassette (which was previously added to the cell-free step) was added directly to this combined annealing/amplification step.

Following the combined annealing/amplification step, 1.5 μl of the SMART reaction sample was added to the cell-free extract assay, which now contained, in addition to the 1.5 μl of SMART reaction, the PURExpress Solution A, PURExpress Solution B, and chlorophenol red-β-D-galactopyranoside substrate components. The resulting A₅₆₀ was measured (FIG. 14) and demonstrated that the SMART reaction and toehold switch combination could detect the target CCPP RNA sequence (SEQ ID NO:23) when the annealing and amplification steps were combined.

1.7.8 Use of DNA Rather than RNA to Produce Signal RNA2.

Previous publications using SMART (Wharam et al., 2001, ibid; Hall et al., 2002, ibid) have used a single-stranded amplification probe to which signal RNA1 anneals. Annealing of signal RNA1 to the amplification probe leads to the generation of a double-stranded amplification probe through the action of DNA polymerase. This then allows signal RNA2 to be transcribed, as shown in FIG. 2D.

An alternative method of amplification would be to produce a single-stranded DNA molecule, that would bind to the amplification probe and cause secondary RNA production. Such a single-stranded DNA molecule could be produced via the nicking strategy shown in FIG. 4.

To demonstrate this, a DNA oligonucleotide (SEQ ID NO:38) was purchased that was an analogue of RND1 signal RNA (SEQ ID NO:35) (named RND1 signal DNA). Either RND1 signal DNA or an in vitro-transcribed RND1 signal RNA were added at varying concentrations in the presence of 83.6 nM amplification probe P24 (SEQ ID NO:39) and, following DNA digestion with Exonuclease V, the amount of RNA2 (SEQ ID NO:36) production was measured by RT-qPCR using primers (SEQ ID NO:7) and (SEQ ID NO:8) (FIG. 15). Strikingly, the RND1 signal DNA resulted in up to 465x amplification at the concentrations tested (0.1 nM-100 nM), whereas RND1 signal RNA (SEQ ID NO:35) gave between 0.9× and 13× amplification at the concentrations tested (6 nM-6000 nM). This data strongly suggests that the mechanism to produce signal DNA rather than signal RNA from a 3WJ will result in a much higher level of amplification and RNA production.

### Example 2 An Example Protocol for Conducting an Assay in the Field

The following is a protocol example for how this type of reaction will be applied in the field when the technology is integrated into a portable cartridge. The general procedure for extracting nucleic acids involves disintegration of cells and pathogen to release nucleic acids (lysis) and separation of nucleic acids from the other cell components. One of the methods to achieve lysis is by using chemical means. Chemical lysis and nucleic acid purification protocols have been commercialised in manual laboratory RNA preparation kits, e.g. QIAamp® Viral RNA Mini Kit (52904, QIAGEN). The process used in commercial kits can be automated by including it into a disposable cartridge. A cartridge is the physical platform that contains the required passive components to process an individual sample to result.

Filters for the retention of large particles or ones for plasma preparation are used to prepare blood samples for the release of nucleic acids using chemicals. Many analytes are found in plasma, and therefore plasma separation procedures are well developed. The sample flow through microfluidic channels within a cartridge are driven by micropumps and the flow of reagents is driven by either micropumps or pouches. Pouches are flexible reagent storage containers located within a cartridge. Deformation of the container results in the release of its contents into the channel network. The timing and direction of flow is controlled by valves. The flow-through from filtration is combined with lysis buffer to achieve highly denaturing conditions for inactivation of RNases and isolation of intact RNA. Lysate mixture then passes through silica membrane and in the presence of certain salts, nucleic acids are retained by adsorption. The columns are then washed with salt solutions to remove unbound particles, and the nucleic acids are finally eluted with water or a low-salt solution. Eluted nucleic acids are then transferred by a micropump to the first chamber where enzymes required for SMART are fixed (e.g. by freeze-drying). Heating element maintains 41° C. temperature and time is given for the SMART reaction to occur. Contents are then transferred to the second chamber where chlorophenol red-β-D-galactopyranoside substrate and the cell-free reaction components containing toehold switch-lacZ DNA cassette are fixed. The heating element maintains a 41° C. temperature and time is given for a colour change reaction to occur. The colour change is then read by eye or automatically if an optical reader is available.

1. A method of detecting a nucleic acid sequence of interest in a sample, the method comprising (a) contacting the sample with first and second probes, capable of creating a three-way junction when the target sequence is present in the sample wherein the first probe comprises a portion substantially complementary to the sequence of interest and so capable of hybridising thereto, and a portion non-complementary to the sequence of interest but comprising a sequence complementary to the second probe and a template signal sequence, and wherein the second probe comprises a portion substantially complementary to the sequence of interest and so capable of hybridising thereto, and a portion non-complementary to the sequence of interest but complementary to a part of that portion of the first probe which is non-complementary to the sequence of interest, such that the first and second probes are capable of hybridising to the sequence of interest in an adjacent or substantially adjacent manner so as to allow complementary portions of the first and second probes to hybridise to each other; (b) causing production of an oligonucleotide sequence that is complementary to the template signal sequence in the first probe; (c) bringing an oligonucleotide trigger sequence into contact with a riboregulator switch sequence, part of which is in the form of a hairpin loop structure, comprising an RNA sequence having single-stranded and double-stranded domains comprising a single-stranded domain capable of hybridising with part of the oligonucleotide trigger sequence, a ribosome binding site (RBS), an initiation codon and a coding domain for a reporter gene arranged such that the ribosome is only able to effect translation when the hairpin loop structure has been disrupted following binding of the oligonucleotide trigger sequence to the riboregulator switch; and (d) detecting the reporter gene product; wherein the presence of the reporter gene product indicates the presence of the sequence of interest in the sample; and, wherein the oligonucleotide trigger sequence is either the oligonucleotide sequence produced in step (b) or an oligonucleotide sequence produced using the oligonucleotide sequence produced in step (b) as an intermediate in an amplification reaction.

2. The method of claim 1, wherein the first probe and/or the second probe comprise a destabilising moiety.

3. The method according to claim 2, wherein the destabilising moiety comprises hexaethylene glycol, pentamethylene or hexamethylene.

4. The method according to claim 1, wherein two further probes, facilitator probe 1 (FP1) and facilitator probe 2 (FP2), are used in step (a), wherein FP1 comprises a sequence capable of hybridising to the target sequence of interest at a site adjacent or substantially adjacent to the annealing site of the first probe and FP2 comprises a sequence capable of hybridising to the target sequence of interest at a site adjacent to or substantially adjacent to the annealing site of the second probe.

5. The method according to claim 1, wherein the oligonucleotide sequence in step (b) is produced directly or indirectly by primer extension of probe 2 using probe 1 as template and a DNA polymerase.

6. The method according to claim 5, wherein the DNA polymerase is a thermophilic DNA polymerase.

7. The method according to claim 6, wherein the thermophilic DNA polymerase is selected from the group consisting of: a Bacillus stearothermophilus (Bst) DNA polymerase, a derivative of 9° N™ DNA Polymerase such as Therminator DNA polymerase and Vent(exo-) DNA polymerase.

8. The method according to claim 1, wherein formation of the three-way junction (3WJ) when the first probe, second probe and target sequence hybridise together or extension of the second probe results in formation of an RNA polymerase promoter.

9. The method according to claim 5, wherein primer extension of the second probe using the first probe as template generates a double-stranded RNA polymerase promoter and RNA signal sequence.

10. The method according to claim 1, wherein formation of the 3WJ when the first probe, second probe and target sequence hybridise together generates a functional double-stranded RNA polymerase promoter.

11. The method according to claim 8, wherein the RNA polymerase promoter is a T3, T7 or SP6 promoter.

12. The method according to claim 5, wherein primer extension of the second probe using the first probe as template generates a double-stranded restriction enzyme recognition sequence.

13. The method according to claim 12, wherein the restriction enzyme recognition sequence is recognised by the restriction enzyme Nb.Bsml.

14. The method according to claim 1, wherein the oligonucleotide trigger sequence is the oligonucleotide sequence generated and released from the 3WJ produced when the first probe, second probe and target sequence hybridise together.

15. The method according to claim 1, wherein the oligonucleotide trigger sequence is a sequence produced when the oligonucleotide sequence produced in step (b) is subjected to an amplification reaction.

16. The method according to claim 1, wherein the oligonucleotide trigger sequence comprises or consists of the sequence disclosed in SEQ ID NO: 35 or 38.

17. The method according to claim 15, wherein the amplification reaction involves contacting the oligonucleotide sequence produced in step (b) with an amplification probe comprising three regions, a first region comprising a sequence sufficiently complementary to the oligonucleotide sequence produced in step (b) to allow hybridisation thereto, a second region encoding the full-length sequence of a first strand of a double-stranded RNA promoter and a third region comprising a first strand of a double-stranded nucleic acid signal sequence, such that extension of the bound oligonucleotide sequence produced in step (b) with a nucleic acid polymerase using the nucleic acid amplification sequence as a template, produces a functional RNA polymerase promoter and double-stranded signal sequence which can then be used by RNA polymerase to produce the oligonucleotide trigger sequence.

18. The method according to claim 17, wherein the amplification probe comprises or consists of the sequence disclosed in SEQ ID NO: 3, 39 or 40.

19. The method according to claim 1, wherein the riboregulator switch comprises a toehold domain.

20. The method according to claim 19, wherein the riboregulator switch comprises a sequence selected from the group consisting of SEQ ID NO: 41, 42, 43 and 45, a sequence with at least 90% sequence identity thereto or a sequence with 1, 2, 3, 4, 5 or 6 substitutions therein.

21. The method according to wherein the reporter gene is fluorescent, luminescent or colourimetric.

22. The method according to claim 21, wherein the reporter gene is a green fluorescent protein (GFP).

23. The method according to claim 21, wherein the reporter gene is LacZ (b-galactosidase) enzyme.

24. The method according to claim 23, wherein the production of LacZ enzyme is detected by contacting with the enzyme substrate chlorophenol red-b-galactopyranoside and detecting colour change.

25. The method according to claim 1, wherein steps (a) to (d) are carried out at the same time.

26. The method according to claim 1, wherein steps (a) and (b) are carried out in a first reaction phase and then the reaction product from this first reaction phase is brought into contact with the toehold switch sequence from step (c) and steps (c) and (d) are carried out in a second reaction phase.

27. The method according to claim 1, wherein all the reagents needed to carry out steps (a) to (d) aside from the sample are present at one or more sites on a solid substrate

28. The method according to claim 27, wherein the solid substrate is plastic, polymer-based, hydrogel, glass, silicon, or paper-based.

29. The method according to claim 28, wherein the method is carried out on paper, card or another paper-based substrate.

30. A solid substrate comprising one or more zones with reagents attached thereon, said reagents comprising: a first probe and a second probe capable of creating a three-way junction with a target sequence of interest and releasing an oligonucleotide signal sequence, and a riboregulator switch molecule.

31. The solid substrate according to claim 30, wherein the substrate comprises a paper-based material.

32. The solid substrate according to claim 30, wherein the substrate is part of a microfluidic device.

33. A kit for use in detecting the presence in a sample of a nucleic acid sequence of interest, the kit comprising the first probe, the second probe and the riboregulator switch molecule in accordance with claim 1.

34. The kit according to claim 33, also comprising one or both facilitator probes in accordance with claim 4.

35. The kit according to claim 33, further comprising instructions for use in performing the method of claim 1.

36. The kit according to claim 33, further comprising one or more of the following: a DNA polymerase, an RNA polymerase; ribo-nucleotide triphosphates, deoxyribo-nucleotide triphosphates; a cell-free system, detection reagents and buffers.

37. A trio of nucleic acid probes, the first probe comprising a portion substantially complementary to the sequence of interest and so capable of hybridising thereto, and a portion non-complementary to the sequence of interest but comprising the full-length sequence of a first strand of a double-stranded RNA promoter and a template signal sequence, the second probe comprising a portion substantially complementary to the sequence of interest and so capable of hybridising thereto, and a portion non-complementary to the sequence of interest but complementary to a part of that portion of the first probe which is non-complementary to the sequence of interest, such that the first and second probes are capable of hybridising to the sequence of interest in an adjacent or substantially adjacent manner, so as to allow complementary portions of the first and second probes to hybridise to each other, and the third probe being a riboregulator switch sequence in a hairpin structure comprising single-stranded and double-stranded domains comprising a single-stranded domain capable of hybridising with some or all of an oligonucleotide trigger sequence, a RBS, an initiation codon and a coding domain for a reporter gene arranged such that the ribosome is only able to effect translation when the hairpin loop structure has been disrupted following binding of the oligonucleotide trigger sequence to the riboregulator switch.

38. A trio of nucleic acid sequences consisting of the first probe, the second probe, and the riboregulator switch sequence in accordance with claim 1.

39. A method of detecting a nucleic acid sequence of interest in a sample, the method comprising (a) contacting the sample with first and second probes capable of hybridising to the nucleic acid sequence of interest and each other to form a three-way junction (3WJ) complex; (b) generating a single-stranded oligonucleotide sequence from the 3WJ; (c) optionally, using the single-stranded oligonucleotide sequence in step (b) to create multiple copies of a single-stranded oligonucleotide trigger sequence; bringing the oligonucleotide produced in step (b) or (c) into contact with a riboregulator switch sequence which comprises a sequence complementary to the single-stranded oligonucleotide sequence produced in step (b) or (c), a RBS, an initiation codon and a reporter gene, wherein upon binding of the oligonucleotide produced in step (b) or (c) to the riboregulator switch sequence the reporter gene product is produced; and (e) detecting the presence of the reporter gene product, wherein the presence of the reporter gene product indicates that the nucleic acid sequence of interest is in the sample.

40. A riboregulator switch molecule which comprises a toehold domain, a RBS, an initiation codon and a reporter gene, wherein the molecule is formed from a single-stranded molecule that is capable of self-hybridising to form regions of single and double strands including a single-stranded toehold domain, a partially or fully double-stranded stem domain, and a single-stranded hairpin loop domain, wherein the RBS is located in the stem domain and wherein binding of an oligonucleotide signal sequence to the toehold domain and a part or all of a stem domain effects a conformational change in the self-annealed riboregulator switch molecule which allows production of the reporter gene product.

41. The riboregulator switch molecule according to claim 40, wherein the toehold domain is upstream of the RBS.

42. The riboregulator switch molecule according to claim 40, wherein the toehold domain is at the 5′ end of the molecule and is single-stranded.

43. The riboregulator switch molecule according to claim 40, which molecule comprises or consists of a sequence selected from the group consisting of SEQ ID NO: 41, 42, 43 and 45, a sequence with at least 90% sequence identity thereto or a sequence with 1, 2, 3, 4, 5 or 6 substitutions therein.


SEQUENCE LISTING (49 sequences)

SEQ ID NO: 1 - RNA, 163 nt, Cyanophage N1
ggguucuacauugauguuggcaaucuuccaaagguaaaagcagaacaauaccucagagagguaaugggacguuaccgcaacaaacuuguuuaugaugcaaacacaggugaaaucaaggacgacaagaaacauaugucgaugcuugcuaguuauugcucagcgg

SEQ ID NO: 2 - DNA, 96 bp, Artificial Sequence
  template probe
  misc_feature (69)..(69): hexaethylenglycol (HEG) moiety
  misc_feature (96)..(96): hexaethylenglycol (HEG) moiety
tcgtcttccggtctctcctctcaagcctcagcgctctctctccctatagtgagtcgtattaatttcgaaacgtcccattacctctctgaggtattg

SEQ ID NO: 3 - DNA, 117 bp, Artificial Sequence
  amplification probe
  misc_feature (82)..(82): hexaethylenglycol (HEG) moiety
  misc_feature (117)..(117): 3′ phosphorylation
tagtataagtaaatcgcttgctgtatgtcgttattctgccgtagggcaccctatagtgagtcgtattaatttctcgtcttccggtctctcctctcaagcctcagcgctctctctccc

SEQ ID NO: 4 - RNA, 42 nt, Artificial Sequence
  Signal RNA
gggcaugaauaacgacauacagcaagcgauuuacuuauacua

SEQ ID NO: 5 - RNA, 92 nt, Artificial Sequence
  Toehold switch B
gggagaaaguaaaucgcuugcuguaugucguuaaacagaggagauaacgaaugacagcaagcaaccuggcggcagcgcaaaagaugcguaaa

SEQ ID NO: 6 - DNA, 3075 bp, Escherichia coli
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 7 - DNA, 15 bp, Artificial Sequence
  RT-qPCR Forward Primer
ggtgccctacggcag

SEQ ID NO: 8 - DNA, 30 bp, Artificial Sequence
  RT-qPCR Reverse Primer
tagtataagtaaatcgcttgctgtatgtcg

SEQ ID NO: 9 - DNA, 20 bp, Artificial Sequence
  PCR Forward Primer (S19)
gaagtctaacgctgctctgg

SEQ ID NO: 10 - DNA, 22 bp, Artificial Sequence
  PCR Reverse Primer (S20)
tctcaaatgcctgaggtttcag

SEQ ID NO: 11 - DNA, 20 bp, Bacteriophage T7
taatacgactcactataggg

SEQ ID NO: 12 - DNA, 20 bp, Bacteriophage T3
aattaaccctcactaaaggg

SEQ ID NO: 13 - DNA, 21 bp, Bacteriophage SP6
aatttaggtgacactatagaa

SEQ ID NO: 14 - DNA, 58 bp, Bacteriophage T5
tcataaaaaatttatttgctttcaggaaaatttttctgtataatagattcataaattt

SEQ ID NO: 15 - DNA, 12 bp, Bacteriophage T7
atcgtcagtccc

SEQ ID NO: 16 - DNA, 12 bp, Bacteriophage T7
gctctctctccc

SEQ ID NO: 17 - DNA, 12 bp, Bacteriophage T7
atcctctctccc

SEQ ID NO: 18 - DNA, 12 bp, Bacteriophage T7
gttctctctccc

SEQ ID NO: 19 - DNA, 8 bp, Bacteriophage T7
agaggaga

SEQ ID NO: 20 - DNA, 38 bp, Artificial Sequence
  Extension probe
gtttgcatcataaacaagtttgttgcggtattcgaaat

SEQ ID NO: 21 - RNA, 60 nt, Bovine viral diarrhoea virus
auggaguugaucacaaaugaacuuuuauacaaaacauacaaacaaaaacccgcuggagug

SEQ ID NO: 22 - RNA, 75 nt, Mycoplasma mycoides
ugaugaaaaaauugacaagccaagucauucagauaaaccacaagcagaugauucuaacaacaauagagacauuuu

SEQ ID NO: 23 - RNA, 57 nt, Mycoplasma capricolum
cacaauucggaguuucacuagauaaaguugaugcuacauuuuuaacaucuccucagc

SEQ ID NO: 24 - RNA, 327 nt, Bos taurus
gggauccugccaacaucaaguggggugaugcuggugcugaguaugugguggaguccacuggggucuucacuaccauggagaaggcuggggcucacuugaaggguggcgccaagagggucaucaucucugcaccuucugccgaugcccccauguuugugaugggcgugaaccacgagaaguauaacaacacccucaagauugucagcaaugccuccugcaccaccaacugcuuggccccccuggccaaggucauccaugaccacuuuggcaucguggagggacuuaugaccacuguccacgccaucacugcuaguuauugcucagcgg

SEQ ID NO: 25 - DNA, 38 bp, Artificial Sequence
  BVDV Extension probe
cactccagcgggtttttgtttgtatgttctagattatg

SEQ ID NO: 26 - DNA, 113 bp, Artificial Sequence
  BVDV Template probe (producing signal RND1 RNA)
  misc_feature (77)..(77): hexaethylenglycol (HEG) moiety
  misc_feature (113)..(113): 3′ phosphorylation
atctgtttccgtcatccttagtccattcccatcatcgtccagttctctctccctatagtgagtcgtattacataatctgtataaaagttcatttgtgatcaactccatttttt

SEQ ID NO: 27 - DNA, 106 bp, Artificial Sequence
  BVDV Template probe (producing signal B RNA)
  misc_feature (75)..(75): hexaethylenglycol (HEG) moiety
  misc_feature (106)..(106): 3′ phosphorylation
tagtataagtaaatcgcttgctgtatgtcgttattctgccgtagggcaccctatagtgagtcgtattacataatctgtataaaagttcatttgtgatcaactccat

SEQ ID NO: 28 - DNA, 108 bp, Artificial Sequence
  BVDV Template probe (producing signal 42_23 RNA)
  misc_feature (77)..(77): hexaethylenglycol (HEG) moiety
  misc_feature (108)..(108): 3′ phosphorylation
ctggaactggatggatgtcattgcgtaaagcctctatgcaccttatggtgccctatagtgagtcgtattacataatctgtataaaagttcatttgtgatcaactccat

SEQ ID NO: 29 - DNA, 42 bp, Artificial Sequence
  CBPP Extension Probe
gtctctattgttgttagaatcatctgcttgtgacggattatg

SEQ ID NO: 30 - DNA, 110 bp, Artificial Sequence
  CBPP Template Probe
  misc_feature (77)..(77): hexaethylenglycol (HEG) moiety
  misc_feature (110)..(110): 3′ phosphorylation
atctgtttccgtcatccttagtccattcccatcatcgtccagttctctctccctatagtgagtcgtattacataatcgtttatctgaatgacttggcttgtcaatttttt

SEQ ID NO: 31 - DNA, 34 bp, Artificial Sequence
  CCPP Extension probe
ggagatgttaaaaatgtagcatcagttgattatg

SEQ ID NO: 32 - DNA, 106 bp, Artificial Sequence
  CCPP Template probe
  misc_feature (77)..(77): hexaethylenglycol (HEG) moiety
  misc_feature (106)..(106): 3′ phosphorylation
atctgtttccgtcatccttagtccattcccatcatcgtccagttctctctccctatagtgagtcgtattacataatcctttatctagtgaaactccgaattgtgaa

SEQ ID NO: 33 - DNA, 40 bp, Artificial Sequence
  GAPDH Extension probe
gttggtggtgcaggaggcattgctgacaatactgattatg

SEQ ID NO: 34 - DNA, 113 bp, Artificial Sequence
  GAPDH Template probe
  misc_feature (77)..(77): hexaethylenglycol (HEG) moiety
  misc_feature (113)..(113): 3′ phosphorylation
atctgtttccgtcatccttagtccattcccatcatcgtccagttctctctccctatagtgagtcgtattacataatccttgagggtgttgttatacttctcgtggtttttttt

SEQ ID NO: 35 - RNA, 53 nt, Artificial Sequence
  Signal RNA (signal RND1)
gggagagagaacuggacgaugaugggaauggacuaaggaugacggaaacagau

SEQ ID NO: 36 - RNA, 51 nt, Artificial Sequence
  Signal RNA (signal B)
gggugcccuacggcagaauaacgacauacagcaagcgauuuacuuauacua

SEQ ID NO: 37 - RNA, 53 nt, Artificial Sequence
  Signal RNA (signal 42_23)
gggcaccauaaggugcauagaggcuuuacgcaaugacauccauccaguuccag

SEQ ID NO: 38 - DNA, 53 bp, Artificial Sequence
  Signal DNA (signal RND1)
gggagagagaactggacgatgatgggaatggactaaggatgacggaaacagat

SEQ ID NO: 39 - DNA, 112 bp, Artificial Sequence
  Amplification Probe P24
  misc_feature (82)..(82): hexaethylenglycol (HEG) moiety
  misc_feature (112)..(112): 3′ phosphorylation
tagtataagtaaatcgcttgctgtatgtcgttattctgccgtagggcaccctatagtgagtcgtattaatttcatctgtttccgtcatccttagtccattcccatcatcgtc

SEQ ID NO: 40 - DNA, 114 bp, Artificial Sequence
  Amplification Probe P80
  misc_feature (84)..(84): hexaethylenglycol (HEG) moiety
  misc_feature (114)..(114): 3′ phosphorylation
ctggaactggatggatgtcattgcgtaaagcctctatgcaccttatggtgccctatagtgagtcgtattaatttcatctgtttccgtcatccttagtccattcccatcatcgtc

SEQ ID NO: 41 - RNA, 90 nt, Artificial Sequence
  Toehold switch 117
gggccuuaguccauucccaucaucguccaaggccucuagacaaugaaacagaggagauggacgaugauaaaccuggcggcagcgcaaaag

SEQ ID NO: 42 - RNA, 92 nt, Artificial Sequence
  Toehold switch 119
gggccuuaguccauucccaucaucguccaguuccucuaacgcccaauaacuagaggagacggacgaugauaaaccuggcggcagcgcaaaag

SEQ ID NO: 43 - RNA, 94 nt, Artificial Sequence
  Toehold switch 121
gggccuuaguccauucccaucaucguccaguuccucuaacaugccgcuaaacuagaggagacggacgaugauaaaccuggcggcagcgcaaaag

SEQ ID NO: 44 - RNA, 92 nt, Artificial Sequence
  Toehold switch B version 2
ggguauaaguaaaucgcuugcuguaugucguuaaacagaggagauaacgaaugacagcaagcaaccuggcggcagcgcaaaagaugcguaaa

SEQ ID NO: 45 - RNA, 113 nt, Artificial Sequence
  Toehold switch 42_23
gggagauaugaacuggauggaugucauugcguaaagccucuauaccgaacgaaacauagaggagauacgcaaugaaacgauacaaccuggcggcagcgcaaaagcaaaguaag

SEQ ID NO: 46 - DNA, 22 bp, Artificial Sequence
  RND1 RT-qPCR Forward Primer
gggagagagaactggacgatga

SEQ ID NO: 47 - DNA, 28 bp, Artificial Sequence
  RND1 RT-qPCR Reverse Primer
atctgtttccgtcatccttagtccattc

SEQ ID NO: 48 - DNA, 23 bp, Artificial Sequence
  42_23 RT-qPCR Forward Primer
caccataaggtgcatagaggctt

SEQ ID NO: 49 - DNA, 24 bp, Artificial Sequence
  42_23 RT-qPCR Reverse Primer
ctggaactggatggatgtcattgc